(19) United States
(12) Patent Application Publication
(10) Pub. No.: US 2026/0337214 A1
(43) Pub. Date: Oct. 29, 2026

(54) IRON BASED MRI PROBES

(71) Applicant: The General Hospital Corporation, Boston, MA (US)

(72) Inventor: Eric M. Gale, Hingham, MA (US)

(21) Appl. No.: 19/122,654

(22) PCT Filed: Oct. 20, 2023
(86) PCT No.: PCT/US2023/035599

§ 371 Date: Apr. 18, 2025

Related U.S. Application Data
(60) Provisional application No. 63/418,461, filed on Oct. 21, 2022.

(51) Int. Cl. C07F 15/02; A61K 49/10; C07C 237/04; C07D 213/38

(52) U.S. Cl. CPC C07F 15/025; A61K 49/106; C07C 237/04; C07D 213/38; C07C 2601/14

(57) ABSTRACT

The present disclosure provides biochemically responsive Fe(II) and Fe(III) complexes useful as magnetic resonance imaging probes for diagnosis and monitoring acute and chronic inflammatory diseases affecting various organs and tissues.

### FEDERALLY SPONSORED RESEARCH OR DEVELOPMENT

This invention was made with Government support under Grant Nos. DK120663 and DK130004 awarded by the National Institutes of Health. The Government has certain rights in the invention.

### TECHNICAL FIELD

This disclosure relates to biochemically responsive magnetic resonance imaging probes useful for diagnosis and monitoring acute and chronic inflammatory diseases affecting various organs and tissues.

### BACKGROUND

Magnetic resonance imaging (“MRI”) technique using various imaging probes allows non-invasive detection, quantification, and mapping pathology of various diseases at the molecular level. Inflammation is a key determinant in many of those diseases, but inflammation in organ systems is difficult to diagnose or monitor without invasive biopsy. In acute and chronic inflammatory disease affecting organs like the liver, kidney, and bowel, many MRI probes fail to aid in effective diagnosis.

### SUMMARY

The present disclosure is based, at least in part, on a realization that a pendant imidate donor group stabilizes a ternary iron-chelate complex of high-spin Fe(III) and a labile water co-ligand at physiologically relevant pH of 7.4. This anionic imidate donor group forms from deprotonation of a neutral carboxamide donor group upon coordination of Fe(III), and represents a strategy for a universal ligand capable to differentially stabilize both Fe(II) and Fe(III) complexes each having physical properties that are desirable for their use as MRI probes. As such, the complexes of this disclosure, capable of switching between the Fe(II) and Fe(III) oxidation states, are useful as MRI contrast agents which generate little signal in normal tissue but a strong, positive MRI signal in inflamed or oxidatively stressed tissues. Deprotonation of a ligand neutral carboxamide-O donor arm to form an anionic imidate donor upon oxidation from Fe(II) to Fe(III) provides means to differentially stabilize Fe(II) complex without losing Fe(III) relaxivity.

The iron complexes within the present claims are advantageously useful to non-invasively diagnose, map, quantify, and monitor oxidative tissue changes that reflect disease activity that could otherwise only be diagnosed using invasive biopsy. For example, the MRI probes of this disclosure are useful for selective detection of inflamed areas in acute and chronic inflammatory diseases affecting liver, kidney, bowel, pancreas, heart, blood vessels, and lungs, as well as in neuroinflammatory disease states. The MRI probes are also useful for quantifying inflammation in the tumor microenvironment as a biomarker for tumor immunogenicity.

In one general aspect, the present disclosure provides a compound of Formula (A):

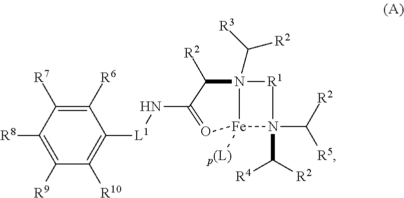

or a pharmaceutically acceptable salt thereof, wherein L, p, R¹-R¹⁰, and L¹ are described herein.

In yet another general aspect, the present disclosure provides a pharmaceutical composition comprising a compound of Formula (A), or a pharmaceutically acceptable salt thereof, and a pharmaceutically acceptable carrier.

In another general aspect, the present disclosure provides a method of magnetic resonance (MR) imaging of an organ or a tissue (e.g., organ or tissue affected by inflammation) of a subject, the method comprising:
i) administering to the subject an effective amount of a compound of Formula (A), or a pharmaceutically acceptable salt thereof, or a pharmaceutical composition comprising same;
ii) waiting a time sufficient to allow the compound of Formula (A) to accumulate in the organ or a tissue (e.g., organ or tissue affected by inflammation) to be imaged; and
ii) acquiring an MR image of the organ or the tissue (e.g., organ or tissue affected by inflammation) of the subject.

In yet another general aspect, the present disclosure provides a method of diagnosing an inflammatory disease or condition in a subject, the method comprising:
i) administering to the subject an effective amount of a compound of Formula (A), or a pharmaceutically acceptable salt thereof, or a pharmaceutical composition comprising same;
ii) waiting a time sufficient to allow the compound to accumulate in an organ or a tissue of the subject affected by the inflammatory disease or condition; and
iii) acquiring an MR image of the organ or the tissue of the subject,
wherein observing the MR image attributable to the compound of Formula (A) is indicative of the inflammatory disease or condition.

In yet another general aspect, the present disclosure provides a method of monitoring treatment of an inflammatory disease or condition in a subject, the method comprising:
i) administering to the subject an effective amount of a compound of Formula (A), or a pharmaceutically acceptable salt thereof, or a pharmaceutical composition comprising same;
ii) waiting a time sufficient to allow the compound to accumulate in an organ or tissue of the subject affected by the inflammatory disease or condition;
iii) acquiring a first MR image of the organ or the tissue of the subject;
iv) administering to the subject a therapeutic agent in an effective amount to treat the inflammatory disease or condition;
v) after iv), administering to the subject an effective amount of a compound of Formula (A), or a pharmaceutically acceptable salt thereof, or a pharmaceutical composition comprising same;
vi) waiting a time sufficient to allow the compound of Formula (A) to accumulate in the organ or tissue of the subject affected by the inflammatory disease or condition;
vii) acquiring a second MR image of the organ or the tissue of the subject; and
viii) comparing the first and the second MR images,
wherein observing a difference between the first and the second MR images attributable to the compound of Formula (A) is indicative of progression of treatment of inflammatory disease or condition.

In yet another general aspect, the present disclosure provides a compound of Formula (B):

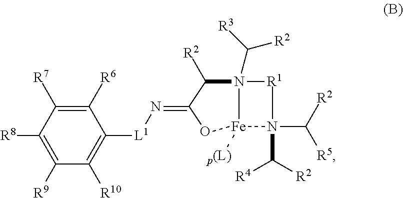

or a pharmaceutically acceptable salt thereof, wherein L, P, L¹ and R¹-R¹⁰ are as described herein.

In yet another general aspect, the present disclosure provides a pharmaceutical composition comprising a compound of Formula (B), or a pharmaceutically acceptable salt thereof, and a pharmaceutically acceptable carrier.

In yet another general aspect, the present disclosure provides a method of magnetic resonance (MR) imaging of an organ or a tissue of a subject, the method comprising:
i) administering to the subject an effective amount of a compound of Formula (B), or a pharmaceutically acceptable salt thereof, or a pharmaceutical composition of comprising same;
ii) waiting a time sufficient to allow the compound to accumulate in the organ or a tissue to be imaged; and
ii) acquiring an MR image of the organ or the tissue of the subject.

In yet another general aspect, the present disclosure provides a method of diagnosing a disease or condition in a subject, the method comprising:
i) administering to the subject an effective amount of a compound of Formula (B), or a pharmaceutically acceptable salt thereof, or a pharmaceutical composition comprising same;
ii) waiting a time sufficient to allow the compound to accumulate in an organ or a tissue of the subject affected by the disease or condition; and
iii) acquiring an MR image of the organ or the tissue of the subject,
wherein observing the MR image attributable to the compound of Formula (B) is indicative of the disease or condition.

In yet another general aspect, the present disclosure provides a method of monitoring treatment of a disease or condition in a subject, the method comprising:
i) administering to the subject an effective amount of a compound of Formula (B), or a pharmaceutically acceptable salt thereof, or a pharmaceutical composition comprising same;
ii) waiting a time sufficient to allow the compound to accumulate in an organ or tissue of the subject affected by the inflammatory disease or condition;
iii) acquiring a first MR image of the organ or the tissue of the subject;
iv) administering to the subject a therapeutic agent in an effective amount to treat the disease or condition;
v) after iv), administering to the subject an effective amount of a compound of Formula (B), or a pharmaceutically acceptable salt thereof, or a pharmaceutical composition comprising same;
vi) waiting a time sufficient to allow the compound to accumulate in the organ or tissue of the subject affected by the disease or condition;
vii) acquiring a second MR image of the organ or the tissue of the subject; and
viii) comparing the first and the second MR images,
wherein observing a difference between the first and the second MR images attributable to the compound of Formula (B) is indicative of progression of treatment of the disease or condition.

In yet another general aspect, the present disclosure provides a compound of Formula (I):

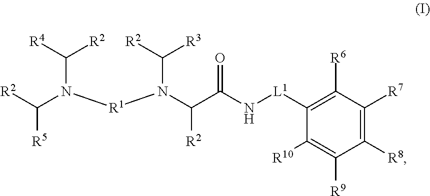

or a pharmaceutically acceptable salt thereof, wherein L¹ and R¹-R¹⁰ are as described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 shows the proposed pH 7.4 solution structure of Fe-CDTA-mA-Ph-3,5-CF₃.

FIG. 2 shows the synthesis of CDTA-mA-Ph-3,5-CF₃ from trans-1,2-diaminocyclohexane-N,N,N′,N′-tetraacetic acid monoanhydride. The experimental details are provided in Example 2, Step 1.

FIG. 3 contains line plots showing pH potentiometric titrations of the exemplified compound Ligand (L) and Complex (ML).

FIG. 4A shows pH-potentiometric titration data of the ligand CDTA-Ph-3,5-CF₃ in the absence (denoted by L) and presence of 1 molar equiv. Fe³⁺ (denoted by ML).

FIG. 4B shows putative structure of the ligand CDTA-Ph-3,5-CF₃ in H₃L form.

FIG. 4C shows structures of the Fe complex of CDTA-Ph-3,5-CF₃ in the ML and MLH₋₁ forms.

FIG. 5A compares the pH-dependence on T₁-relaxivity (r₁; the Fe induced increase in T1 relaxation rate (R1) normalized to Fe concentration in mM) for the structurally related Fe³⁺ complexes Fe-CDTA-Ph-3,5-CF₃ and Fe-PyCy2AI.

FIG. 5B shows chemical equilibria that govern speciation of either complex within the range of measured pH values.4, but how imidate formation stabilizes, high-spin, high-relaxivity mononuclear Fe(III).

FIG. 6 plots the reduced relaxation rate (R₂ᵣ; defined as the Fe induced R₂ increase vs neat water, normalized to the mole fraction of water molecules coordinated to Fe) as a function of temperature.

FIG. 7 compares the relaxivity (r₁; the Fe induced increase in T1 relaxation rate (R1) normalized to Fe concentration in mM) recorded for Fe³⁺-CDTA-Ph-3,5-CF₃ and Fe²⁺-CDTA-Ph-3,5-CF₃ at pH 7.4.

FIG. 8 shows cyclic voltammograms (CV) of Fe-CDTA-Ph-3,5-CF₃ recorded at pH 3 (where the Fe³⁺ complex is in predominantly ML speciation, and pH 9, where the Fe³⁺ complex is in the predominantly MLH₋₁ form.

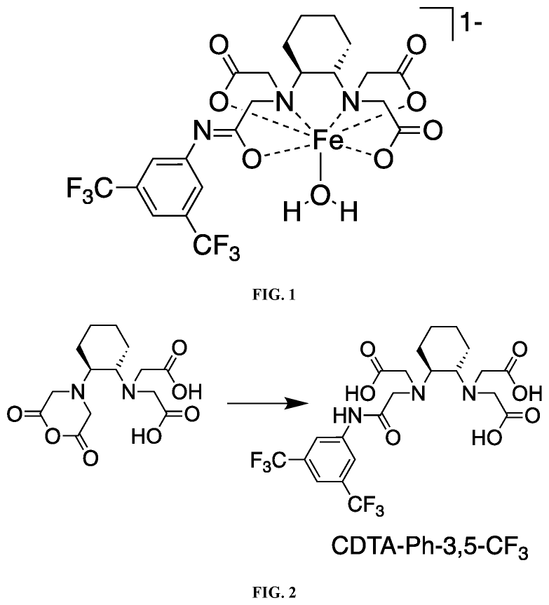

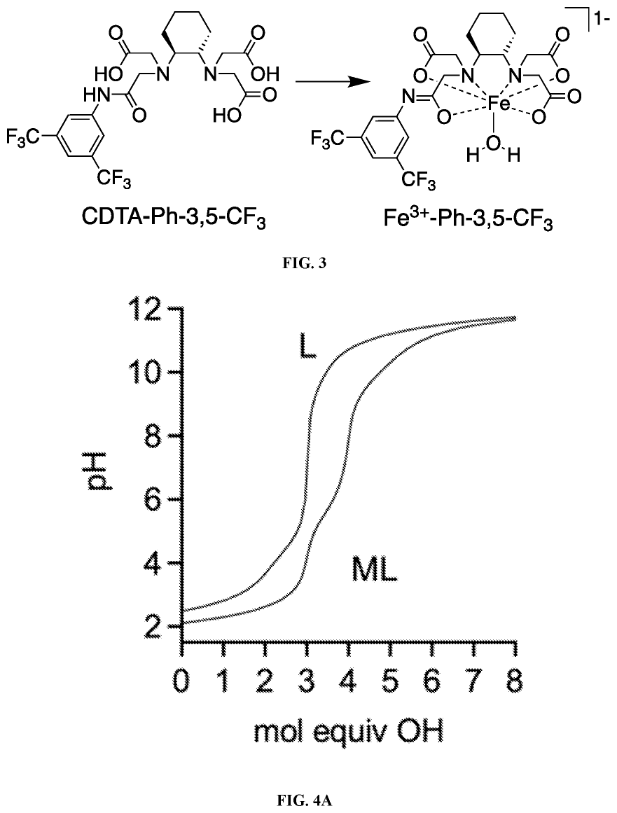

### DETAILED DESCRIPTION

Magnetic resonance imaging (MRI) using biochemically responsive imaging probes offers a potentially powerful approach to non-invasively detect, quantify, and map pathology at the molecular level. Biochemically responsive MR imaging probes can be detected via T₁-relaxation, chemical exchange saturation transfer (CEST), or direct nuclear observation. The Gd complexes are commonly pursued as biochemically responsive T₁-relaxation agents as they are detected with high sensitivity at concentrations that are safely achieved in vivo. However, the biochemical response from Gd-based relaxation agents is often limited by poor dynamic range. Gd complexes possess high relaxivity (r₁) even prior to probe activation and because MR signal in T₁-weighted images in vivo reflects both r₁ and probe concentration, it is challenging to develop Gd-based probes where r₁ is large enough so that interpretation of MR signal response is not confounded by uncertainty in tissue concentration. Probes that modulate MR signal through biochemically triggered changes in the chemical exchange saturation transfer (CEST) effect or ¹H or ¹⁹F chemical shift can provide an “off/on” effect, but low detection sensitivity is a major barrier to use.

Accordingly, the present disclosure advantageously provides biochemically responsive MR relaxation agents as MRI imaging probes where the biological stimuli promotes a large change in the probe's relaxivity. In one example, disclosed herein are stable iron complexes that switch between the Fe(II) and Fe(III) oxidation states. At physiologically relevant pH values near pH 7.4, many structurally Fe(III) complexes dimerize to antiferromagnetically coupled μ-oxo-bridged dimers, that possess very low relaxivity. However, the Fe(III) complexes disclosed herein are designed to exist in predominantly monomeric high-spin state at pH 7.4, providing high relaxivity that enables a large MRI signal turn on effect upon switching from the Fe(II) to Fe(III) oxidation state.

### Compounds of Formula (I)

Provided herein are chelating ligands useful for preparing metal chelates having, e.g., high relaxivity. In some embodiments, the present disclosure provides a ligand compound of Formula (I):

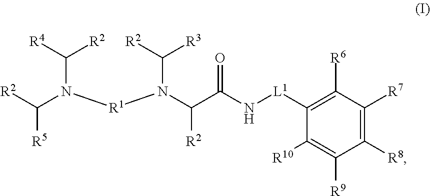

or a pharmaceutically acceptable salt thereof, wherein:
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)OR^{A}, P(O)(OR^{A})(OR^{B}), (C═O)NR^{A}R^{B}; and ring B, which is optionally substituted with 1, 2, or 3 substituents independently selected from R^{1B}.

R⁴ is selected from C(O)OR^{A}, P(O)(OR^{A})(OR^{B}), (C═O)NR^{A}R^{B}; and ring C, which is optionally substituted with 1, 2, or 3 substituents independently selected from R^{1C};
R⁵ is selected from CO(OR^{A}), P(O)(OR^{A})(OR^{B}), (C═O)NR^{A}R^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}SO₂NR^{A}R^{B}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.

In some embodiments, the compound of Formula (I) is not:

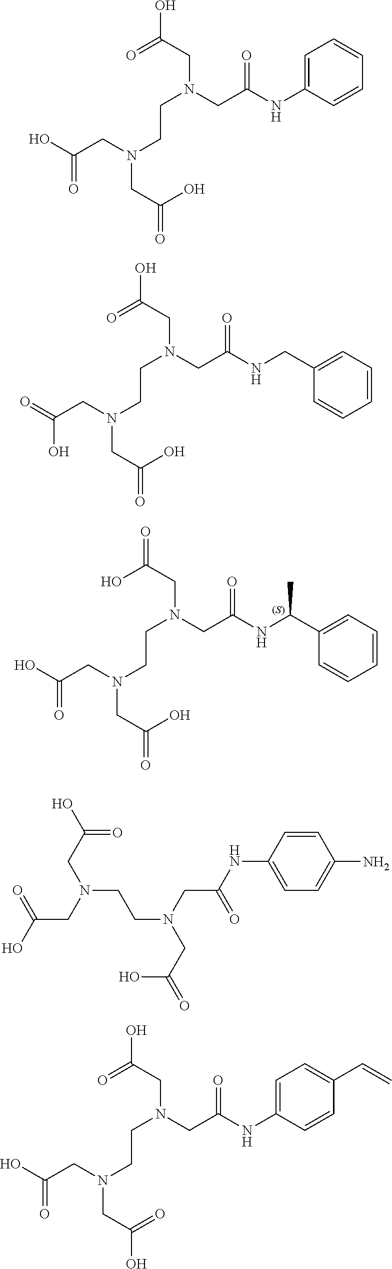

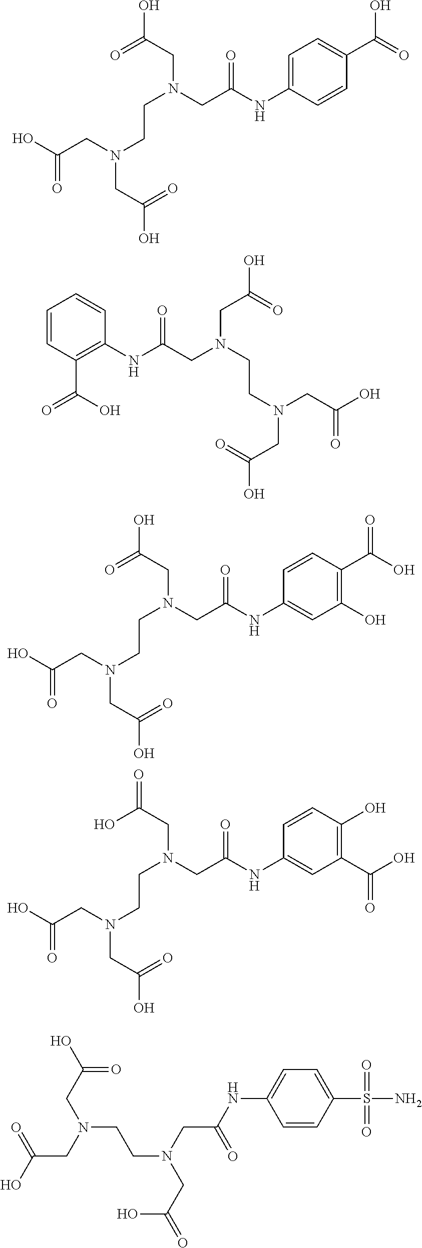

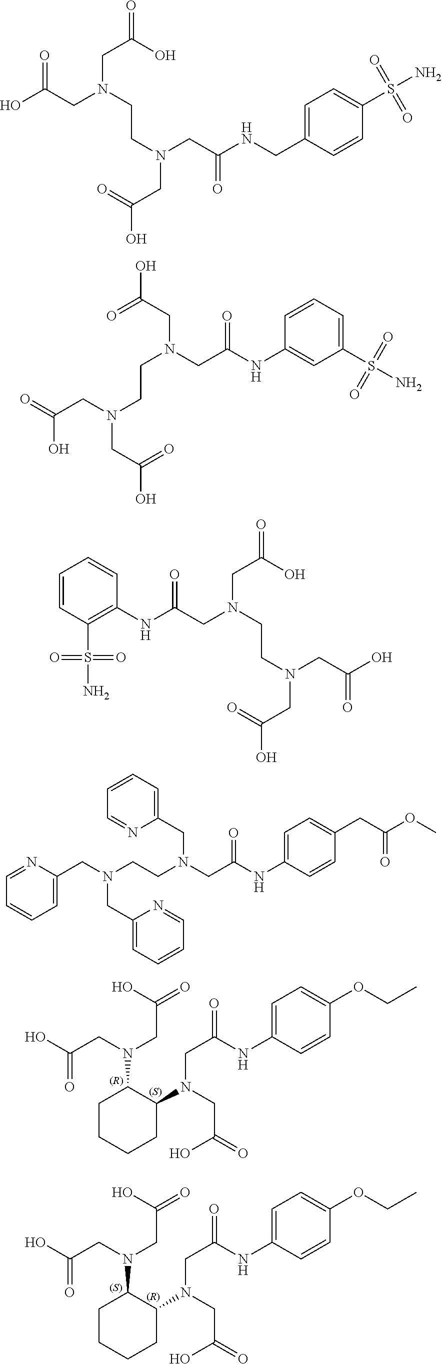

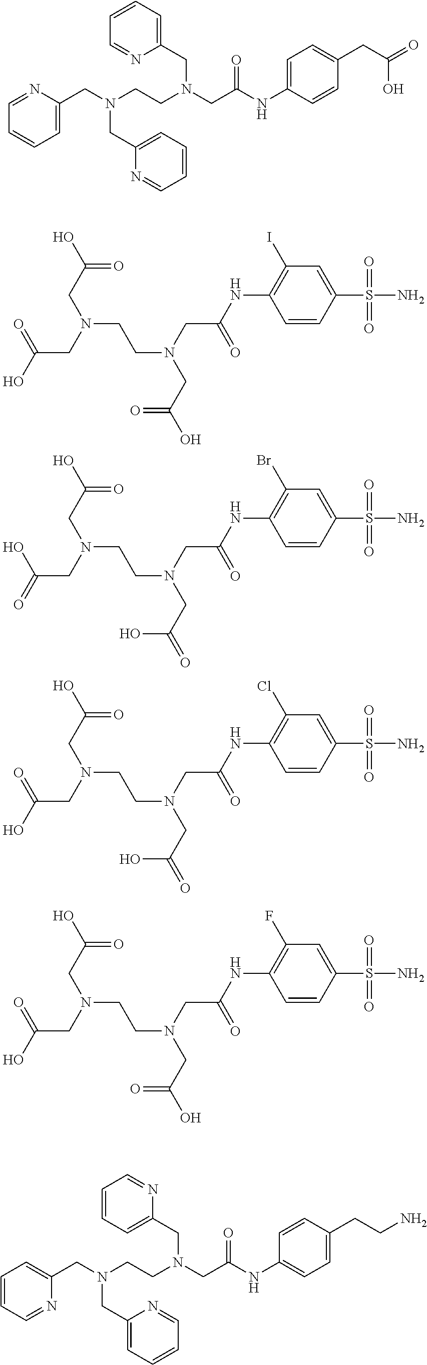 [SMILES: O=C(O)Cc1ccc(NC(=O)CN(CCN(Cc2ccccn2)Cc2ccccn2)Cc2ccccn2)cc1 ; NS(=O)(=O)c1ccc(NC(=O)CN(CCN(CC(=O)O)CC(=O)O)CC(=O)O)c(I)c1 ; NS(=O)(=O)c1ccc(NC(=O)CN(CCN(CC(=O)O)CC(=O)O)CC(=O)O)c(Br)c1 ; NS(=O)(=O)c1ccc(NC(=O)CN(CCN(CC(=O)O)CC(=O)O)CC(=O)O)c(Cl)c1 ; NS(=O)(=O)c1ccc(NC(=O)CN(CCN(CC(=O)O)CC(=O)O)CC(=O)O)c(F)c1 ; NCCc1ccc(NC(=O)CN(CCN(Cc2ccccn2)Cc2ccccn2)Cc2ccccn2)cc1]

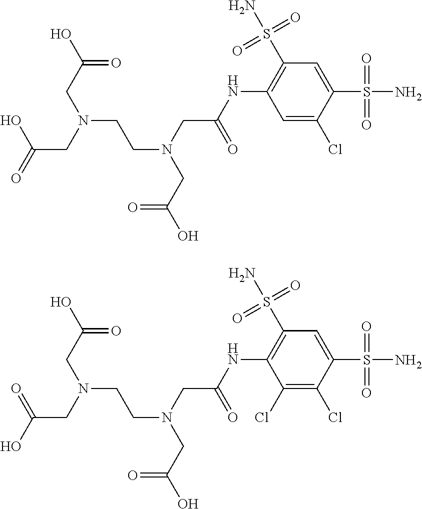 [SMILES: NS(=O)(=O)c1cc(S(N)(=O)=O)c(NC(=O)CN(CCN(CC(=O)O)CC(=O)O)CC(=O)O)cc1Cl ; NS(=O)(=O)c1cc(S(N)(=O)=O)c(NC(=O)CN(CCN(CC(=O)O)CC(=O)O)CC(=O)O)c(Cl)c1Cl]

In some embodiments, the compound of Formula (I) is not:

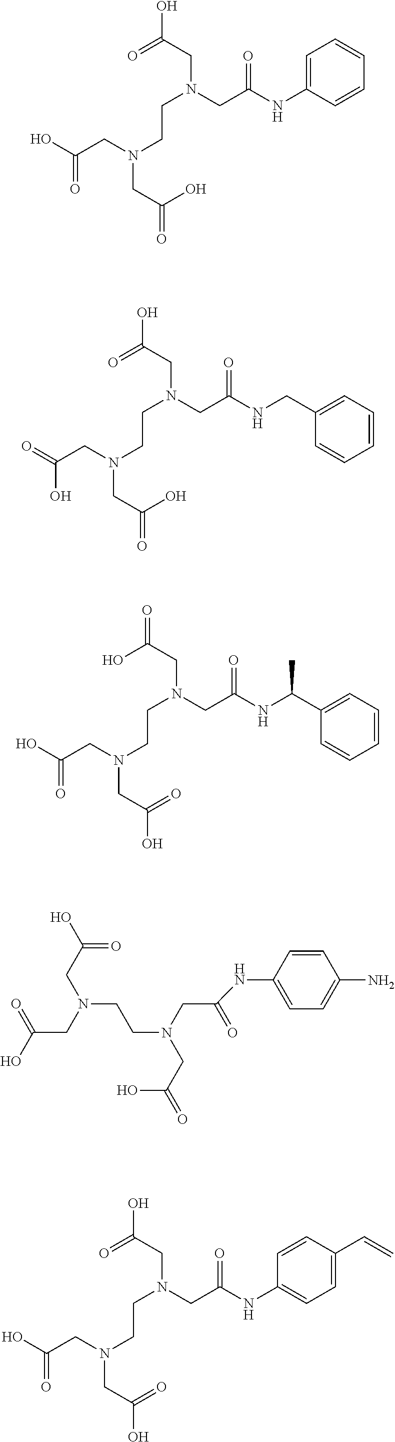 [SMILES: O=C(O)CN(CCN(CC(=O)O)CC(=O)Nc1ccccc1)CC(=O)O ; O=C(O)CN(CCN(CC(=O)O)CC(=O)NCc1ccccc1)CC(=O)O ; C[C@H](NC(=O)CN(CCN(CC(=O)O)CC(=O)O)CC(=O)O)c1ccccc1 ; Nc1ccc(NC(=O)CN(CCN(CC(=O)O)CC(=O)O)CC(=O)O)cc1 ; C=Cc1ccc(NC(=O)CN(CCN(CC(=O)O)CC(=O)O)CC(=O)O)cc1]

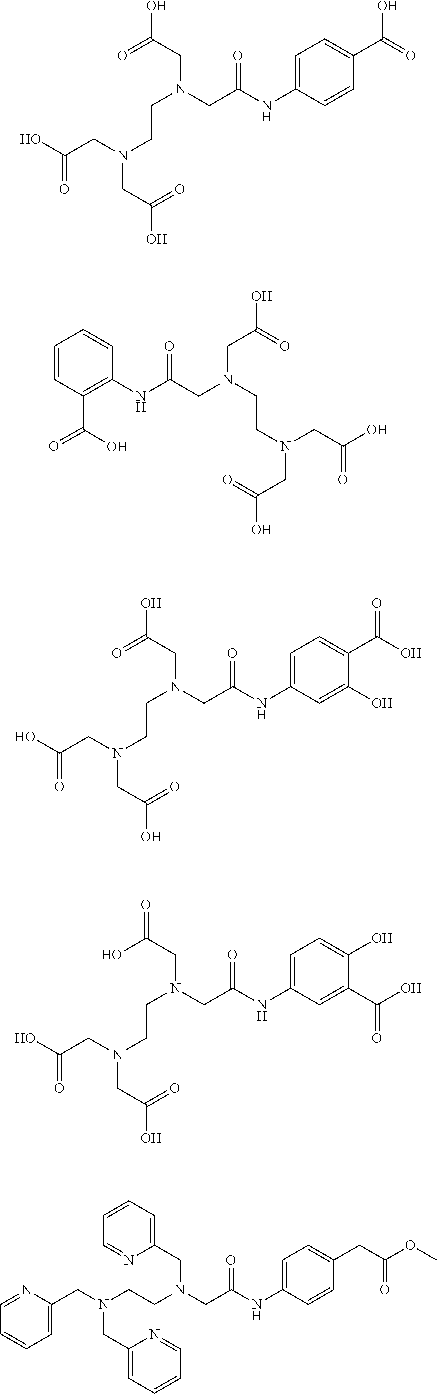 [SMILES: O=C(O)CN(CCN(CC(=O)O)CC(=O)Nc1ccc(C(=O)O)cc1)CC(=O)O ; O=C(O)CN(CCN(CC(=O)O)CC(=O)Nc1ccccc1C(=O)O)CC(=O)O ; O=C(O)CN(CCN(CC(=O)O)CC(=O)Nc1ccc(C(=O)O)c(O)c1)CC(=O)O ; O=C(O)CN(CCN(CC(=O)O)CC(=O)Nc1ccc(O)c(C(=O)O)c1)CC(=O)O ; COC(=O)Cc1ccc(NC(=O)CN(CCN(Cc2ccccn2)Cc2ccccn2)Cc2ccccn2)cc1]

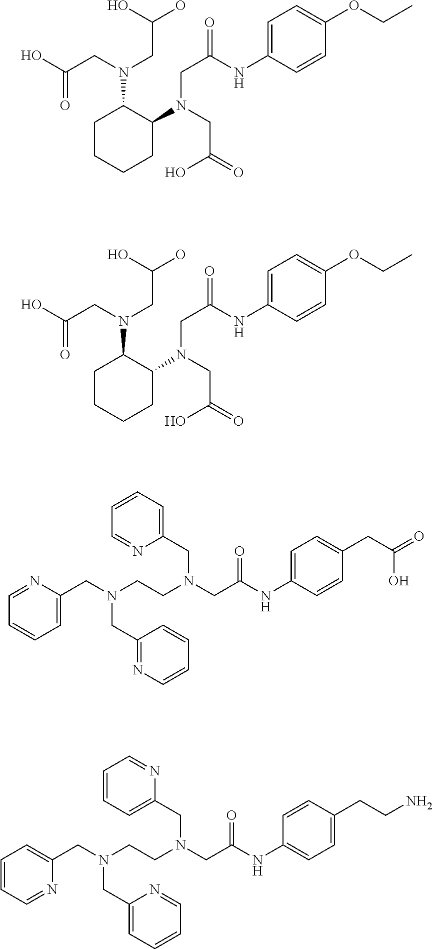 [SMILES: CCOc1ccc(NC(=O)CN(CC(=O)O)[C@H]2CCCC[C@@H]2N(CC(=O)O)CC(O)O)cc1 ; CCOc1ccc(NC(=O)CN(CC(=O)O)[C@@H]2CCCC[C@H]2N(CC(=O)O)CC(O)O)cc1 ; O=C(O)Cc1ccc(NC(=O)CN(CCN(Cc2ccccn2)Cc2ccccn2)Cc2ccccn2)cc1 ; NCCc1ccc(NC(=O)CN(CCN(Cc2ccccn2)Cc2ccccn2)Cc2ccccn2)cc1]

In some embodiments, if R¹ is 1,2-ethylene, then:
(i) R⁶-R¹⁰ are not each H;
(ii) R⁶ is other than carboxy when R⁸ is H; and
(iii) R⁸ is not amino, vinyl, carboxy, OH, CH₂COOCH₃, CH₂COOH, or CH₂CH₂NH₂.

In some embodiments, if R¹ is cyclohexylene, then R¹ is not C₁₋₆ alkoxy (e.g., not ethoxy).

In some embodiments, L¹ is C₁₋₃ alkylene.

In some embodiments, L¹ is methylene.

In some embodiments, L¹ is absent.

In some embodiments, R¹ is C₁₋₃ alkylene-ring A-C₁₋₃ alkylene, wherein said ring A is optionally substituted with 1, 2, or 3 substituents independently selected from R^{1A}.

In some embodiments, the compound has formula:

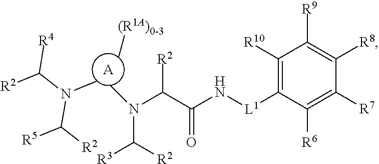

or a pharmaceutically acceptable salt thereof.

In some embodiments, ring A is phenylene, optionally substituted with R^{1A}.

In some embodiments, ring A is 5-6 membered heteroarylene, optionally substituted with R^{1A}.

In some embodiments, ring A is C₃-C₁₀ cycloalkylene, optionally substituted with R^{1A}.

In some embodiments, ring A is cyclopentylene or cyclohexylene, optionally substituted with R^{1A}.

In some embodiments, ring A is a moiety of formula:

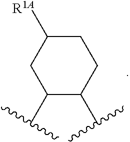

In some embodiments, ring A is 4-10 membered heterocycloalkylene, optionally substituted with R^{1A}.

In some embodiments, R¹ is C₂₋₆ alkylene, optionally substituted with R^{1A}.

In some embodiments, R¹ is 1,2-ethylene, optionally substituted with R^{1A}.

In some embodiments, R¹ is a moiety of formula:

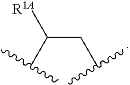

In some embodiments, each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, ring A is selected from any one of the following moieties:

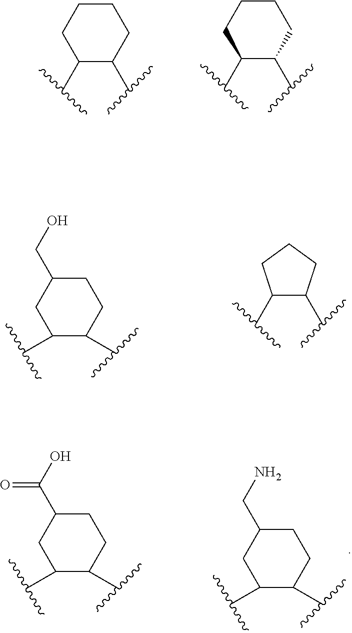 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

In some embodiments, ring A is selected from any one of the following moieties:

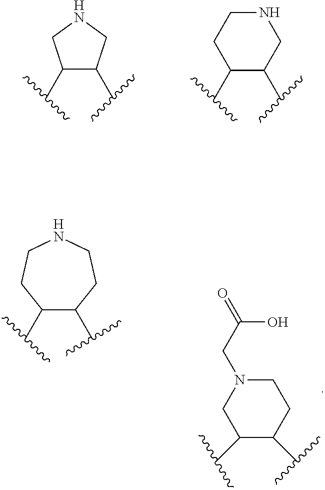 [SMILES: *C1CNCC1* ; *C1CCNCC1* ; *C1CCNCCC1* ; *C1CCN(CC(=O)O)CC1*]

In some embodiments, R¹ is selected from any one of the following moieties:

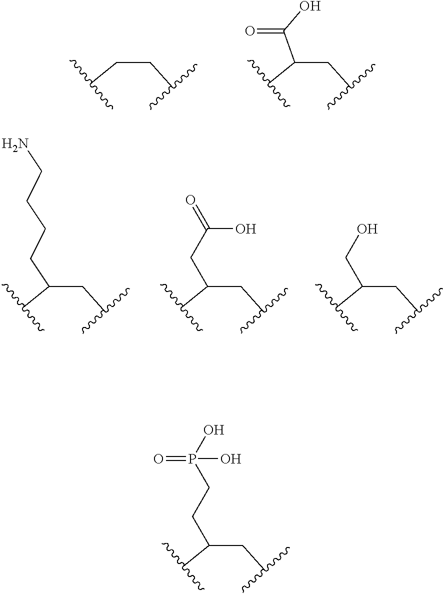

In some embodiments, R¹ is selected from the group consisting of a substituted or unsubstituted alkylene, such as 1,3-propylene or 1,2-ethylene, as shown below:

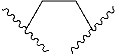 [SMILES: *CC*]

or 2,3-propylene-1-carboxylate, as shown below,

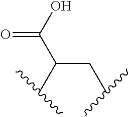 [SMILES: *CC(*)C(=O)O]

or 3,4-butylene-1-carboxylic acid, as shown below,

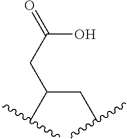 [SMILES: *CC(*)CC(=O)O]

or 1-hydroxy-3,4-butylene, as shown below,

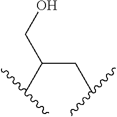 [SMILES: *CC(*)CO]

or 1-amino-5,6-hexylene, as shown below,

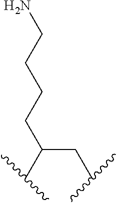 [SMILES: *CC(*)CCCCN]

substituted or unsubstituted cycloalkylene, such as those shown below, for example, cis- or trans-1,2-cyclohexylene, as shown below,

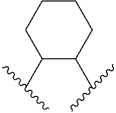 [SMILES: *C1CCCCC1*]

or trans-1,2-cyclohexylene, as shown below,

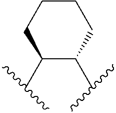 [SMILES: *[C@H]1CCCC[C@@H]1*]

or cis- or trans-1,2-cyclopentylene, as shown below,

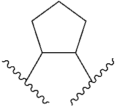 [SMILES: *C1CCCC1*]

substituted or unsubstituted monocyclic heterocyclyl, such as 2,5-dihydro-1H-pyrrolene, as shown below

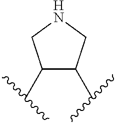 [SMILES: *C1CNCC1*]

1,2,3,6-tetrahydropyridinene, as shown below,

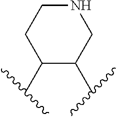 [SMILES: *C1CCNCC1*]

2,3,6,7-tetrahydro-1H-azepinene, as shown below,

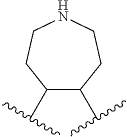 [SMILES: *C1CCNCCC1*]

In some embodiments, each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, each R² is H.

In some embodiments, at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, the compound has formula:

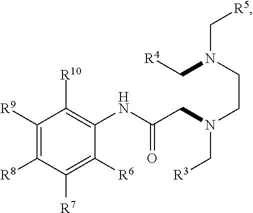

or a pharmaceutically acceptable salt thereof.

In some embodiments, the compound has formula:

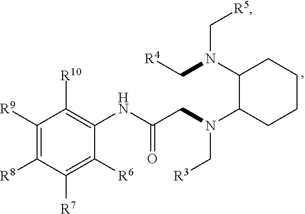

or a pharmaceutically acceptable salt thereof.

In some embodiments, R³ is ring B, optionally substituted with 1, 2, or 3 substituents independently selected from R^{1B}.

In some embodiments, R⁴ is ring C, optionally substituted with 1, 2, or 3 substituents independently selected from R^{1C}.

In some embodiments, R⁵ is ring D, optionally substituted with 1, 2, or 3 substituents independently selected from R^{1D}.

In some embodiments, ring B is 5-6 membered heteroaryl optionally substituted with 1, 2, or 3 substituents independently selected from R^{1B}.

In some embodiments, C is 5-6 membered heteroaryl optionally substituted with 1, 2, or 3 substituents independently selected from R^{1C}.

In some embodiments, ring D is 5-6 membered heteroaryl optionally substituted with 1, 2, or 3 substituents independently selected from R^{1D}.

In some embodiments, the 5-6 membered heteroaryl of ring B, ring C, or ring D is selected from any one of the following moieties:

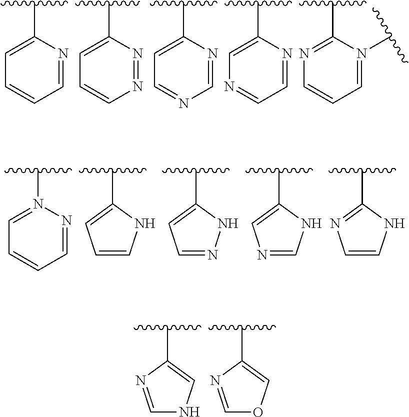

In some embodiments, ring B is 5-6 membered heterocycloalkyl, optionally substituted with 1, 2, or 3 substituents independently selected from R^{1B}.

In some embodiments, ring C is 5-6 membered heterocycloalkyl optionally substituted with 1, 2, or 3 substituents independently selected from R^{1C}.

In some embodiments, ring D is 5-6 membered heterocycloalkyl optionally substituted with 1, 2, or 3 substituents independently selected from R^{1D}.

In some embodiments, the 5-6 membered heterocycloalkyl of ring B, ring C, or ring D is selected from any one of the following moieties:

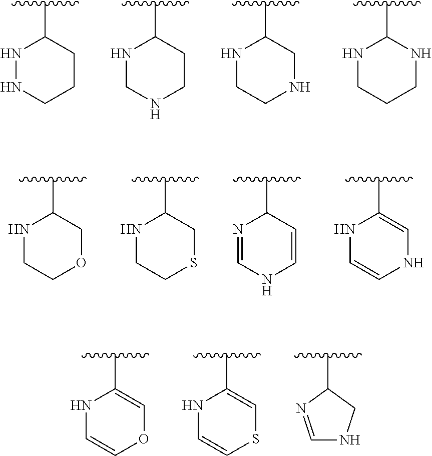

In some embodiments, each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1D} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, the 5-6 membered heteroaryl of ring B, ring C, or ring D is selected from any one of the following moieties:

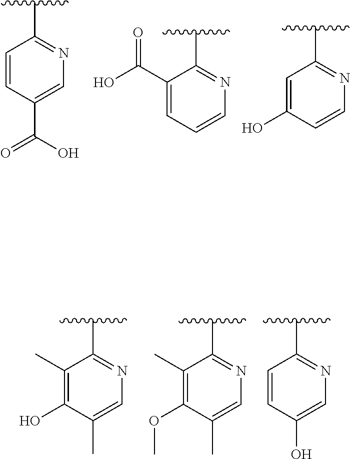

In some embodiments, R³ is C(O)OR^{A}. In some embodiments, R³ is P(O)(OR^{A})(OR^{B}). In some embodiments, R³ is (C═O)NR^{A}R^{B}. In some embodiments, R⁴ is C(O)OR^{A}. In some embodiments, R⁴ is P(O)(OR^{A})(OR^{B}). In some embodiments, R⁴ is (C═O)NR^{A}R^{B}. In some embodiments, R⁵ is C(O)OR^{A}. In some embodiments, R⁵ is P(O)(OR^{A})(OR^{B}). In some embodiments, R⁵ is (C═O)NR^{A}R^{B}.

In some embodiments, the compound of Formula (I) has formula:

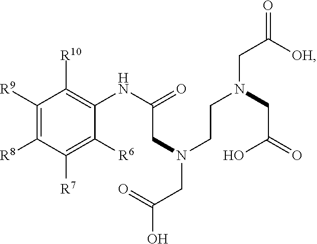

or a pharmaceutically acceptable salt thereof.

In some embodiments, the compound of Formula (I) has formula:

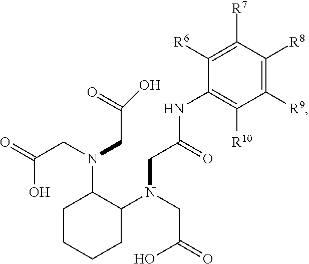

or a pharmaceutically acceptable salt thereof.

In some embodiments R⁶, R⁷, R⁸, R⁹, and R¹⁰ are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from halo, C₁₋₆ haloalkyl, C₁₋₆ haloalkoxy.

In some embodiments, at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, R⁶, R⁷, R⁸, R⁹, and R¹⁰ are independently selected from NO₂, CN, C₁₋₆ alkyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(CH₂)₁₋₃CO₂R^{A}, —(CH₂)₃NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};

In some embodiments:
R⁶, R⁸, and R¹⁰ are each H, and
R⁷ and R⁹ are each independently selected from halo, C₁₋₆ haloalkyl, and C₁₋₆ haloalkoxy.

In some embodiments, R⁷ and R⁹ are each independently selected from halo, C₁₋₆ haloalkyl, and C₁₋₆ haloalkoxy. In some embodiments, R⁷ and R⁹ are each independently C₁₋₆ haloalkyl.

In some embodiments, the compound of Formula (I) has formula:

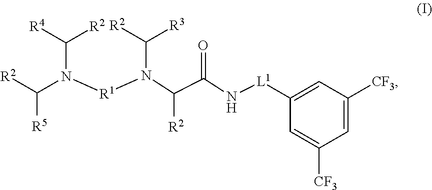

or a pharmaceutically acceptable salt thereof.

In some embodiments, the compound of Formula (I) has formula:

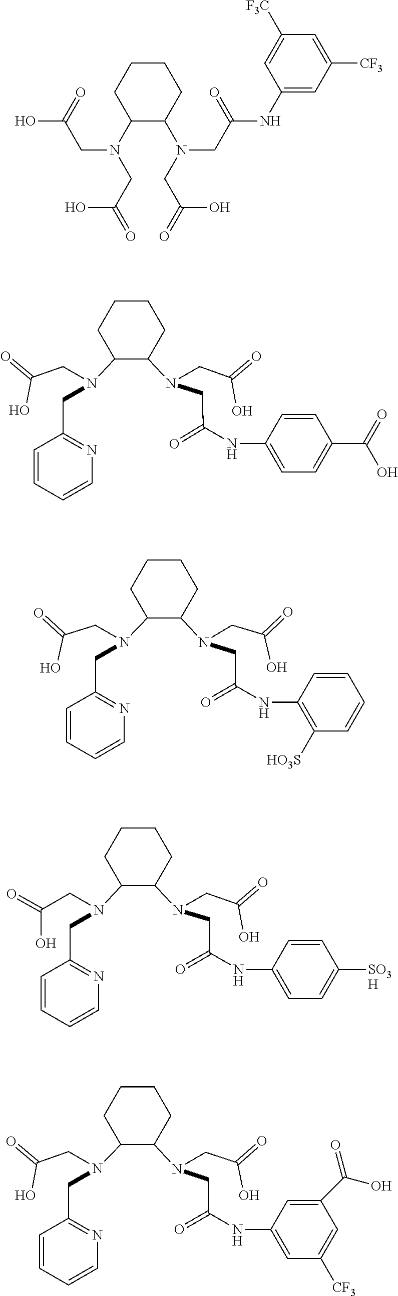

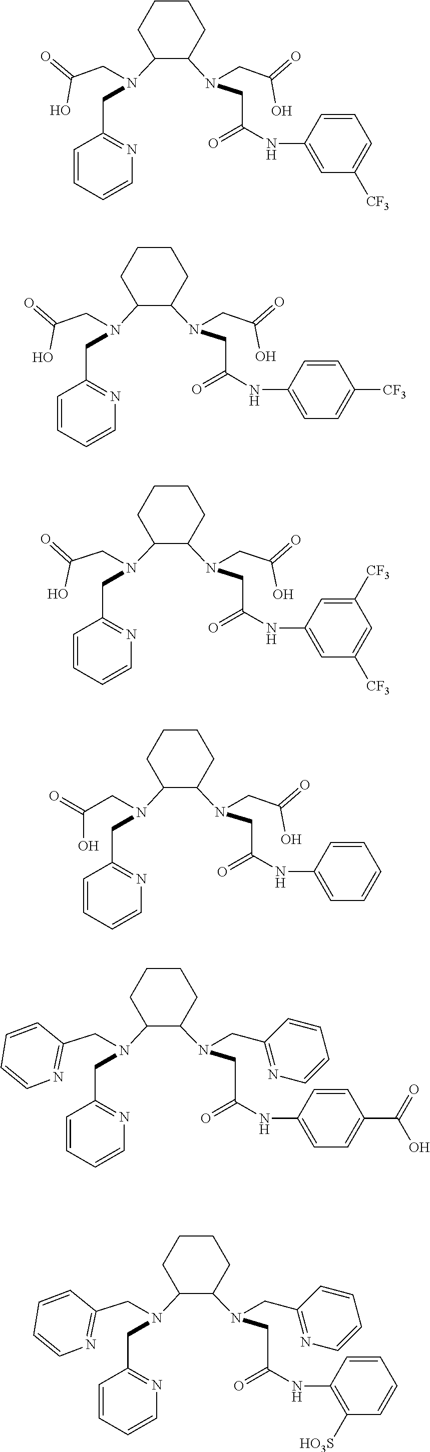

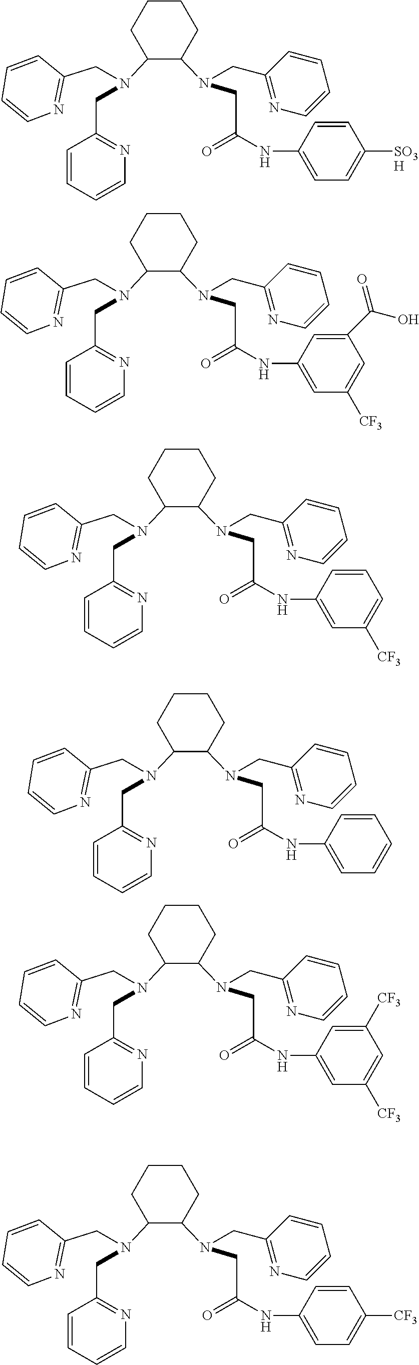

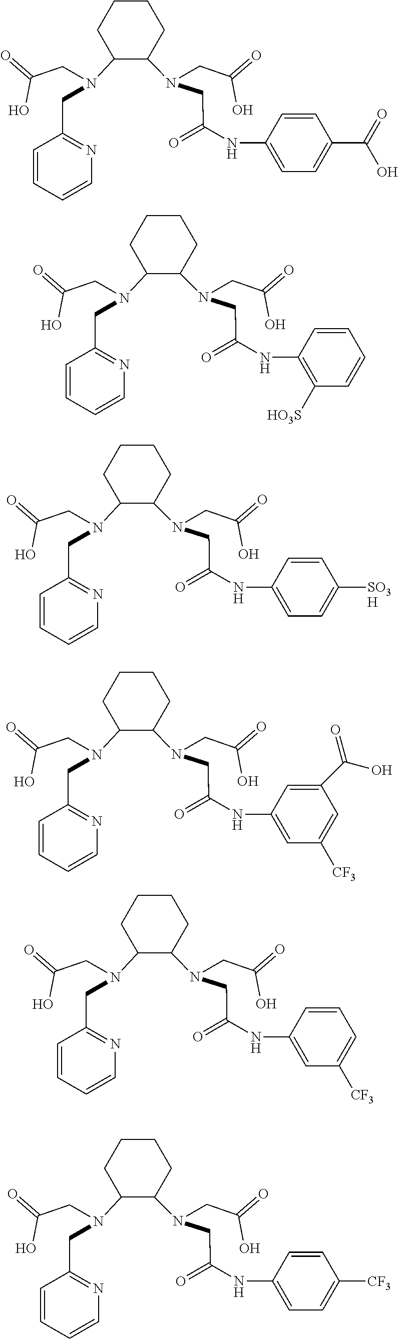

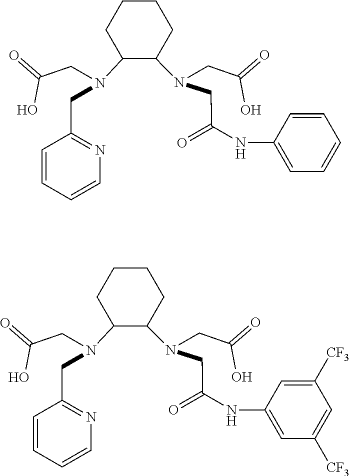

or a pharmaceutically acceptable salt thereof.

In some embodiments, the compound of Formula (I) has any one of the following formulae:

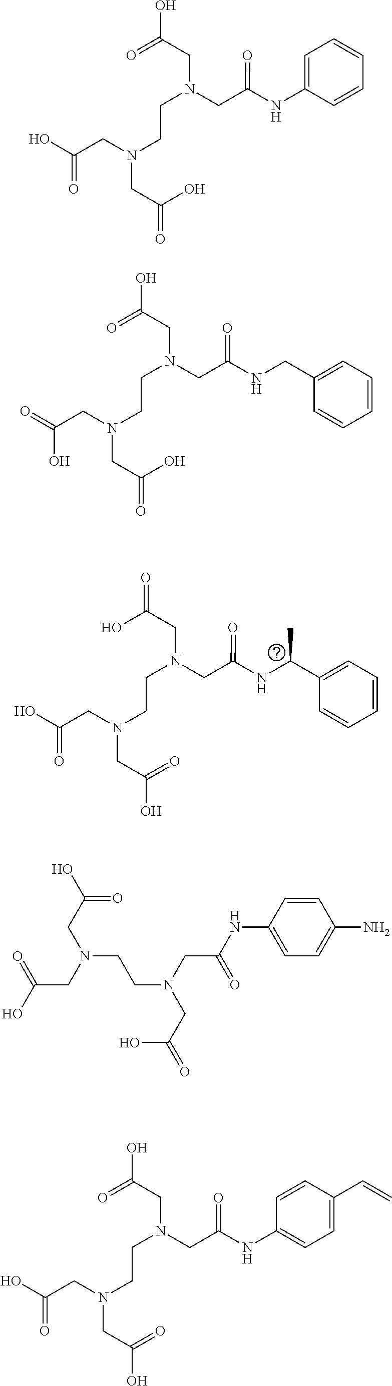

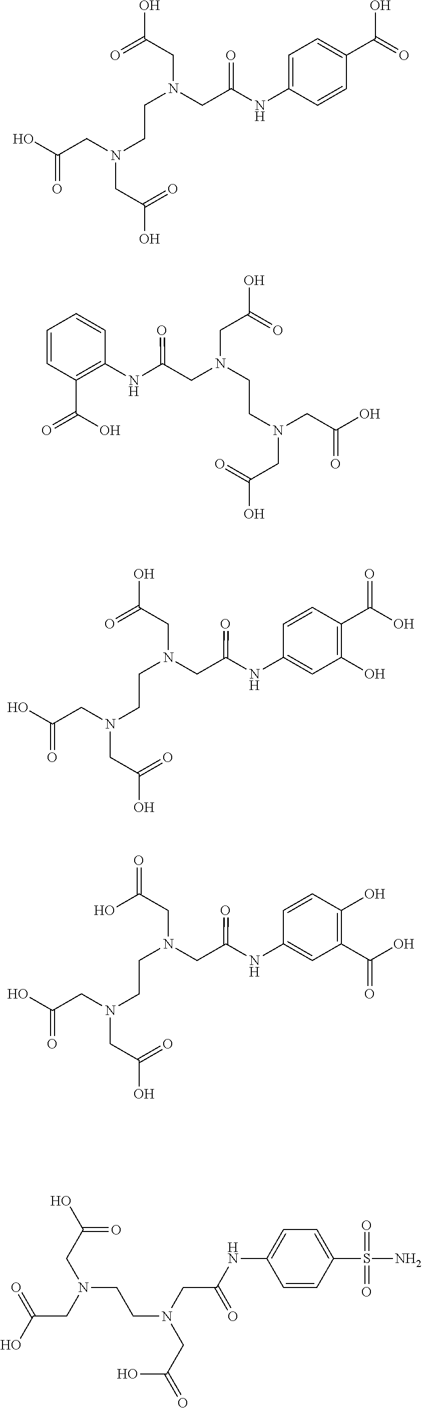 [SMILES: O=C(O)CN(CCN(CC(=O)O)CC(=O)Nc1ccc(C(=O)O)cc1)CC(=O)O ; O=C(O)CN(CCN(CC(=O)O)CC(=O)Nc1ccccc1C(=O)O)CC(=O)O ; O=C(O)CN(CCN(CC(=O)O)CC(=O)Nc1ccc(C(=O)O)c(O)c1)CC(=O)O ; O=C(O)CN(CCN(CC(=O)O)CC(=O)Nc1ccc(O)c(C(=O)O)c1)CC(=O)O ; NS(=O)(=O)c1ccc(NC(=O)CN(CCN(CC(=O)O)CC(=O)O)CC(=O)O)cc1]

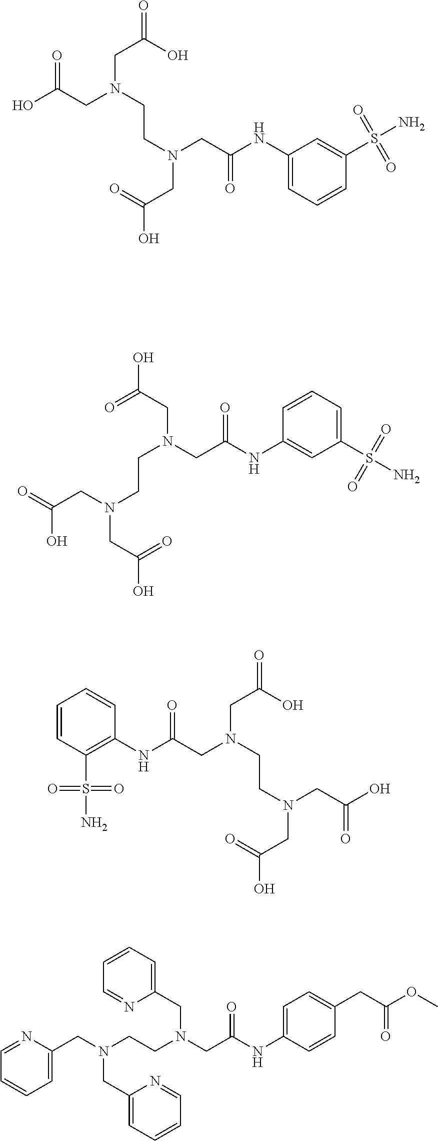 [SMILES: NS(=O)(=O)c1cccc(NC(=O)CN(CCN(CC(=O)O)CC(=O)O)CC(=O)O)c1 ; NS(=O)(=O)c1cccc(NC(=O)CN(CCN(CC(=O)O)CC(=O)O)CC(=O)O)c1 ; NS(=O)(=O)c1ccccc1NC(=O)CN(CCN(CC(=O)O)CC(=O)O)CC(=O)O ; COC(=O)Cc1ccc(NC(=O)CN(CCN(Cc2ccccn2)Cc2ccccn2)Cc2ccccn2)cc1]

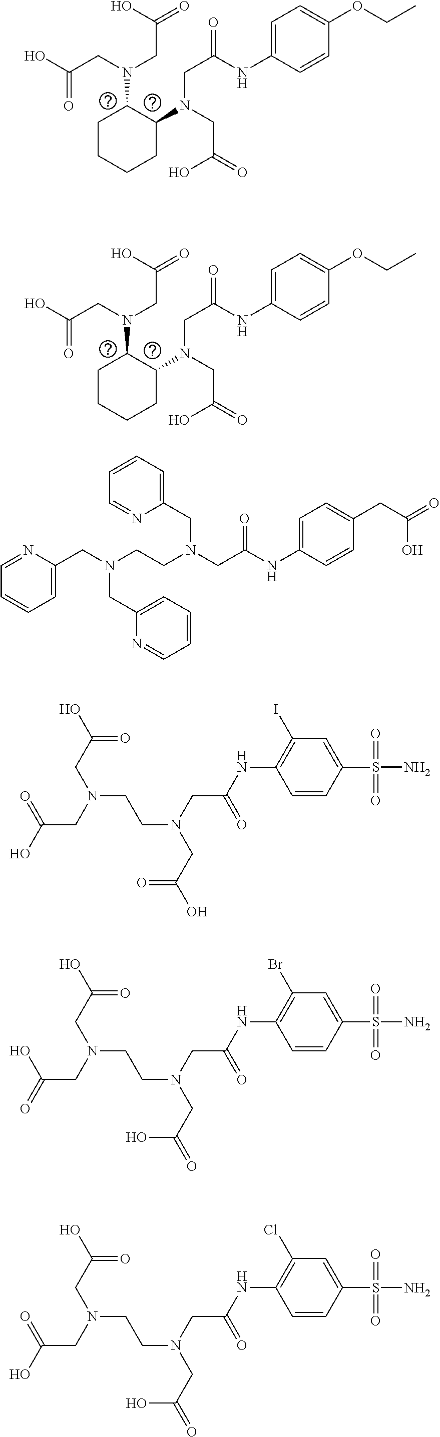

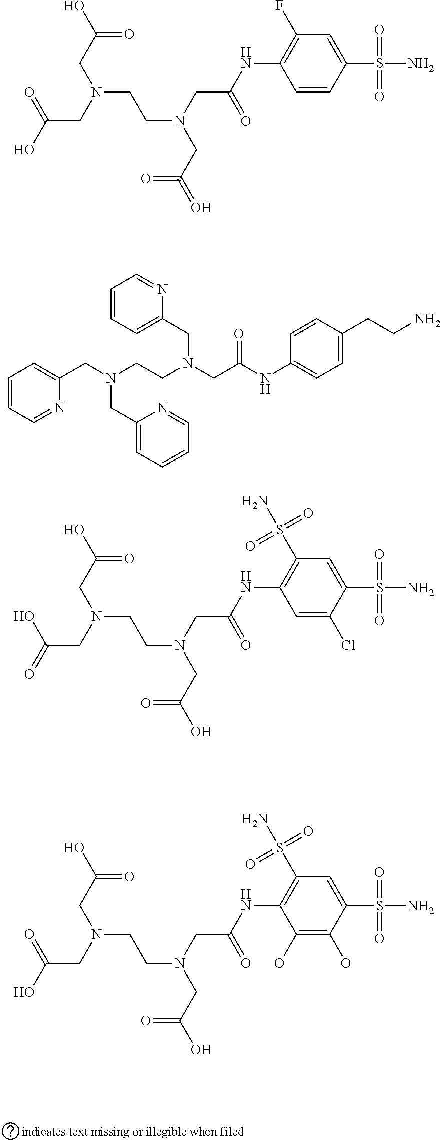

or a pharmaceutically acceptable salt thereof.

### Compounds of Formula (A) and Formula (B)

In some embodiments, the present disclosure provides a coordination complex of Fe²⁺ or Fe³⁺ and a compound of Formula (I). In some embodiments, the complex comprises Fe³⁺. In some embodiments, the complex comprises Fe²⁺. In some embodiments, the complex comprises a combination of Fe²⁺ and Fe³⁺. In some embodiments, the coordination complex further comprises at least one additional iron ligand, in addition to the ligand compound of Formula (I). In some embodiments, each additional iron ligand is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof. In some embodiments, each additional iron ligand is a group L as described hereinbelow. In some embodiments, each L is independently selected from H₂O and NH₃. In some embodiments, each L is H₂O. In some embodiments, L is an inorganic anion such as Cl, Br, SO₄, HCO₃, CO₃, PO₄, nitrate, nitrite, or the like. In some embodiments, each L is an acid or a base commonly present in the plasma. Examples of such L include organic acids such as citric acid, tartaric acid, ascorbic acid, malic acid, succinic acid, or acetic acid. In some embodiments, L is a sugar such as glucose, fructose, mannose, or galactose. In some embodiments, any two L may be joined together to form a single iron ligand. In some embodiments, L is an acid or a base described herein in the “pharmaceutically acceptable salts” section, or an anion or a cation thereof.

In some embodiments, the present disclosure provides a compound of Formula (A):

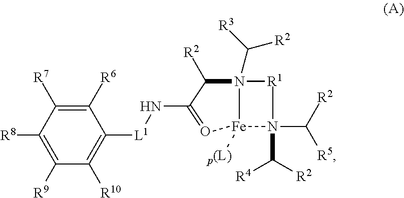

or a pharmaceutically acceptable salt thereof, wherein:
[symbol] indicates a coordinate bond;
each L is independently a Fe ligand;
p is 0, 1 or 2;
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered (I)heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.

In some embodiments, the compound of Formula (A) is not:

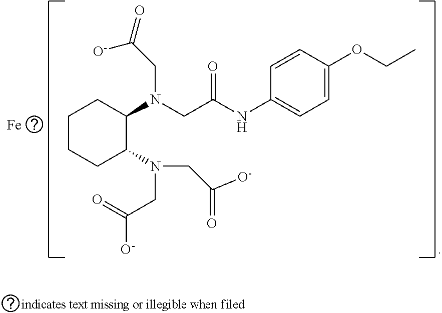

In some embodiments, L¹ is C₁₋₃ alkylene. In some embodiments, L¹ is methylene.

In some embodiments, L¹ is absent.

In some embodiments,

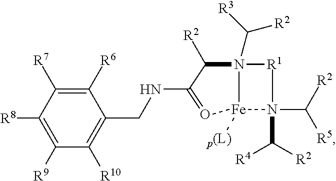

or a pharmaceutically acceptable salt thereof.

In some embodiments,

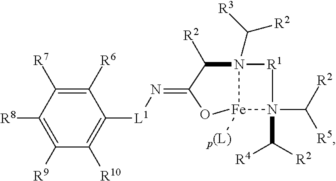

or a pharmaceutically acceptable salt thereof.

In some embodiments, R¹ is C₁₋₃ alkylene-ring A-C₁₋₃ alkylene, wherein said ring A is optionally substituted with 1, 2, or 3 substituents independently selected from R^{1A}.

In some embodiments, the compound has formula:

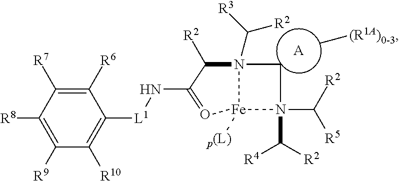

or a pharmaceutically acceptable salt thereof.

In some embodiments, ring A is phenylene, optionally substituted with R^{1A}.

In some embodiments, ring A is 5-6 membered heteroarylene, optionally substituted with R^{1A}.

In some embodiments, ring A is C₃-C₁₀ cycloalkylene, optionally substituted with R^{1A}. In some embodiments, ring A is cyclopentylene or cyclohexylene, optionally substituted with R^{1A}.

In some embodiments, ring A is a moiety of formula:

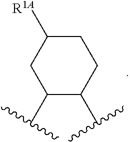

In some embodiments, ring A is 4-10 membered heterocycloalkylene, optionally substituted with R^{1A}.

In some embodiments, R¹ is C₂₋₆ alkylene, optionally substituted with R^{1A}.

In some embodiments, R¹ is 1,2-ethylene, optionally substituted with R^{1A}.

In some embodiments, R¹ is a moiety of formula:

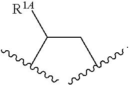

In some embodiments, each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, ring A is selected from any one of the following moieties:

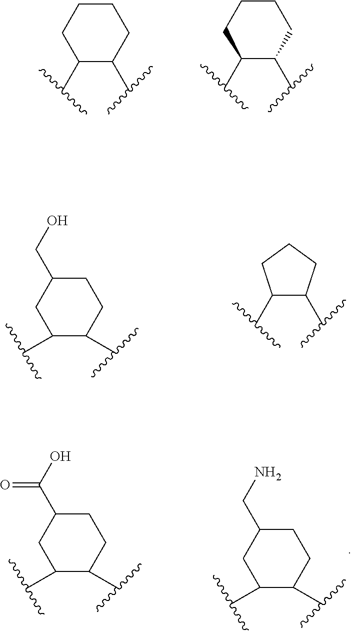 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

In some embodiments, ring A is selected from any one of the following moieties:

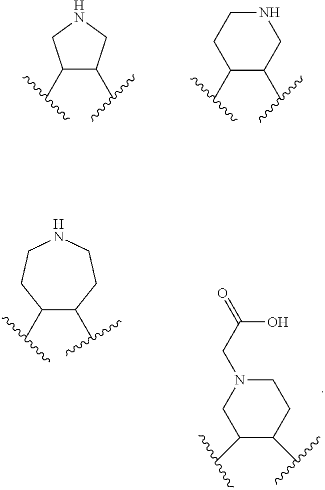 [SMILES: *C1CNCC1* ; *C1CCNCC1* ; *C1CCNCCC1* ; *C1CCN(CC(=O)O)CC1*]

In some embodiments, R¹ is selected from any one of the following moieties:

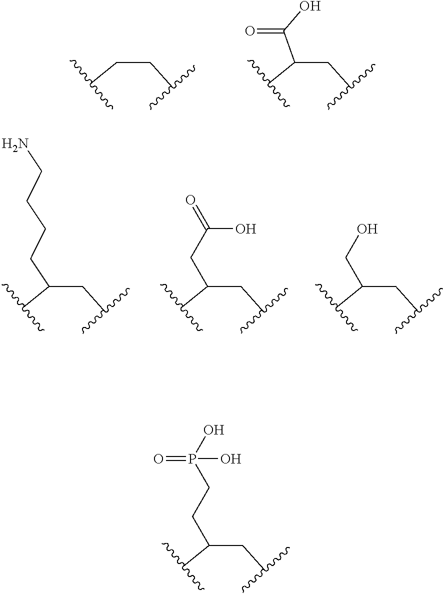

In some embodiments, R¹ is selected from the group consisting of a substituted or unsubstituted alkylene, such as 1,3-propylene or 1,2-ethylene, as shown below:

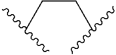 [SMILES: *CC*]

or 2,3-propylene-1-carboxylate, as shown below,

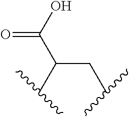 [SMILES: *CC(*)C(=O)O]

or 3,4-butylene-1-carboxylic acid, as shown below,

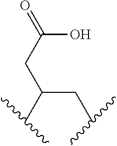 [SMILES: *CC(*)CC(=O)O]

or 1-hydroxy-3,4-butylene, as shown below,

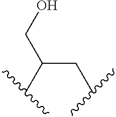 [SMILES: *CC(*)CO]

or 1-amino-5,6-hexylene, as shown below,

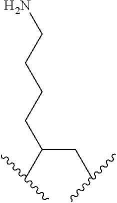 [SMILES: *CC(*)CCCCN]

substituted or unsubstituted cycloalkylene, such as those shown below, for example, cis- or trans-1,2-cyclohexylene, as shown below,

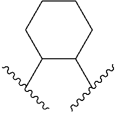 [SMILES: *C1CCCCC1*]

or trans-1,2-cyclohexylene, as shown below,

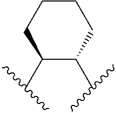 [SMILES: *[C@H]1CCCC[C@@H]1*]

or cis- or trans-1,2-cyclopentylene, as shown below,

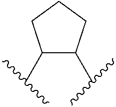 [SMILES: *C1CCCC1*]

substituted or unsubstituted monocyclic heterocyclyl, such as 2,5-dihydro-1H-pyrrolene, as shown below

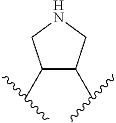 [SMILES: *C1CNCC1*]

1,2,3,6-tetrahydropyridinene, as shown below,

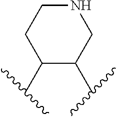 [SMILES: *C1CCNCC1*]

2,3,6,7-tetrahydro-1H-azepinene, as shown below,

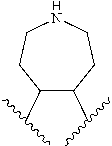 [SMILES: *C1CCNCCC1*]

In some embodiments, each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, each R² is H.

In some embodiments, at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, the compound has formula:

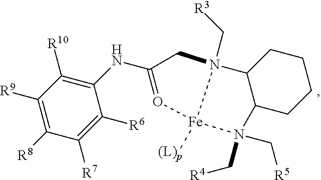

or a pharmaceutically acceptable salt thereof.

In some embodiments, the compound has formula:

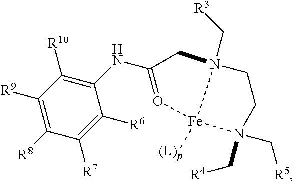

or a pharmaceutically acceptable salt thereof.

In some embodiments, R³ is ring B, optionally substituted with 1, 2, or 3 substituents independently selected from R^{1B}.

In some embodiments, R⁴ is ring C, optionally substituted with 1, 2, or 3 substituents independently selected from R^{1C}.

In some embodiments, R⁵ is ring D, optionally substituted with 1, 2, or 3 substituents independently selected from R^{1D}.

In some embodiments, ring B is 5-6 membered heteroaryl optionally substituted with 1, 2, or 3 substituents independently selected from R^{1B}.

In some embodiments, ring C is 5-6 membered heteroaryl optionally substituted with 1, 2, or 3 substituents independently selected from R^{1C}.

In some embodiments, ring D is 5-6 membered heteroaryl optionally substituted with 1, 2, or 3 substituents independently selected from R^{1D}.

In some embodiments, the 5-6 membered heteroaryl of ring B, ring C, or ring D is selected from any one of the following moieties:

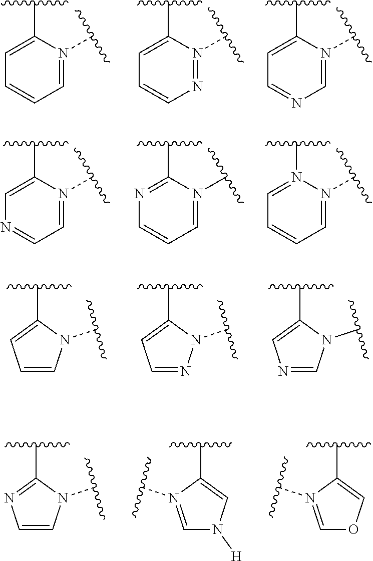

In some embodiments, ring B is 5-6 membered heterocycloalkyl, optionally substituted with 1, 2, or 3 substituents independently selected from R^{1B}.

In some embodiments, ring C is 5-6 membered heterocycloalkyl optionally substituted with 1, 2, or 3 substituents independently selected from R^{1C}.

In some embodiments, ring D is 5-6 membered heterocycloalkyl optionally substituted with 1, 2, or 3 substituents independently selected from R^{1D}.

In some embodiments, the 5-6 membered heterocycloalkyl of ring B, ring C, or ring D is selected from any one of the following moieties:

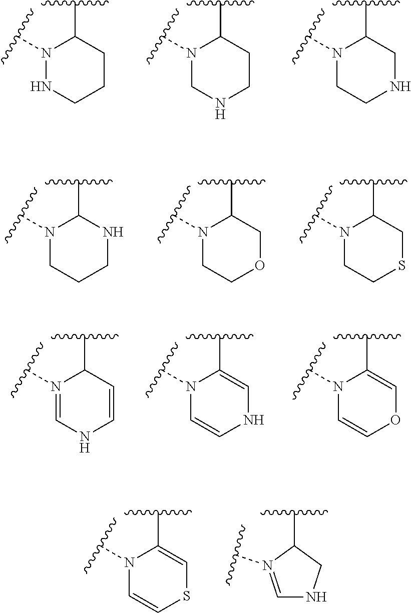

In some embodiments, each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1D} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, the 5-6 membered heteroaryl of ring B, ring C, or ring D is selected from any one of the following moieties:

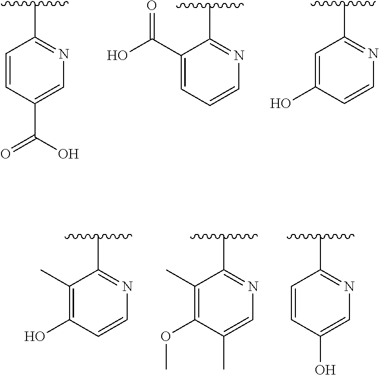

In some embodiments, R³ is C(O)O. In some embodiments, R³ is P(O)(O)(OR^{B}).

In some embodiments, R³ is (C═O)NR^{B}. In some embodiments, R⁴ is C(O)O. In some embodiments, R⁴ is P(O)(O)(OR^{B}). In some embodiments, R⁴ is (C═O)NR^{B}. In some embodiments, R⁵ is C(O)O. In some embodiments, R⁵ is P(O)(O)(OR^{B}). In some embodiments, R⁵ is (C═O)NR^{B}.

In some embodiments, the compound of Formula (A) has formula:

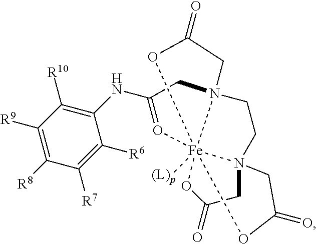

or a pharmaceutically acceptable salt thereof.

In some embodiments, the compound of Formula (A) has formula:

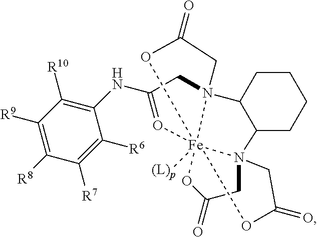

or a pharmaceutically acceptable salt thereof.

In some embodiments, R⁶, R⁷, R⁸, R⁹, and R¹⁰ are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from halo, C₁₋₆ haloalkyl, C₁₋₆ haloalkoxy.

In some embodiments, at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments:
R⁶, R⁸, and R¹⁰ are each H, and
R⁷ and R⁹ are each independently selected from halo, C₁₋₆ haloalkyl, and C₁₋₆ haloalkoxy.

In some embodiments, R⁷ and R⁹ are each independently selected from halo, C₁₋₆ haloalkyl, and C₁₋₆ haloalkoxy. In some embodiments, R⁷ and R⁹ are each independently C₁₋₆ haloalkyl.

In some embodiments, the compound of Formula (A) has formula:

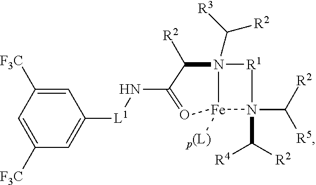

or a pharmaceutically acceptable salt thereof.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof. In some embodiments, p is 1 and L is H₂O.

In some embodiments, the compound of Formula (A) has formula:

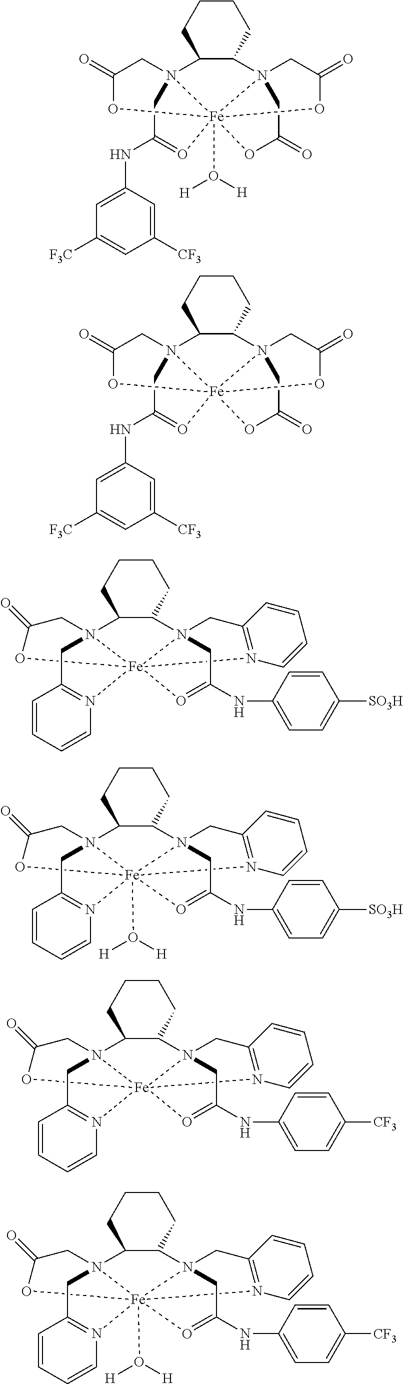

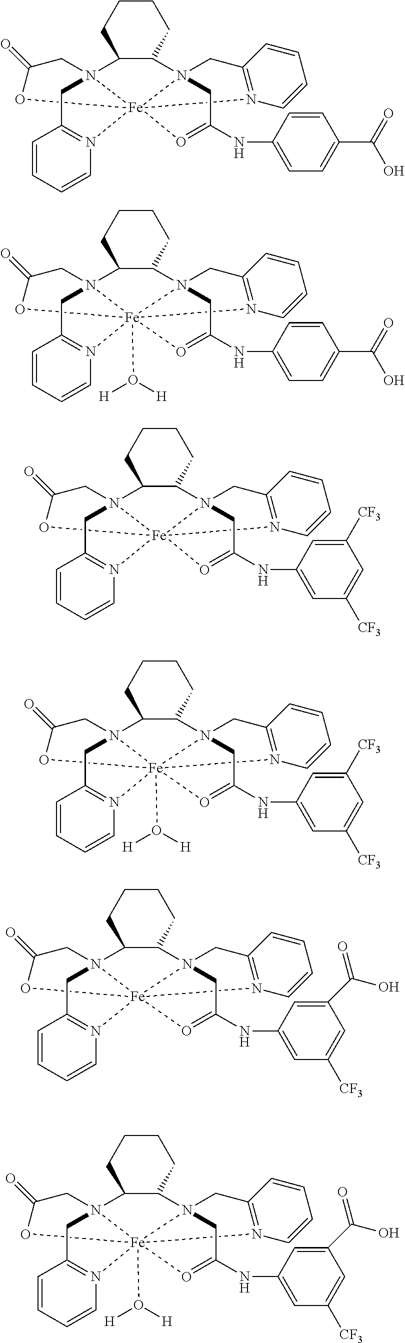

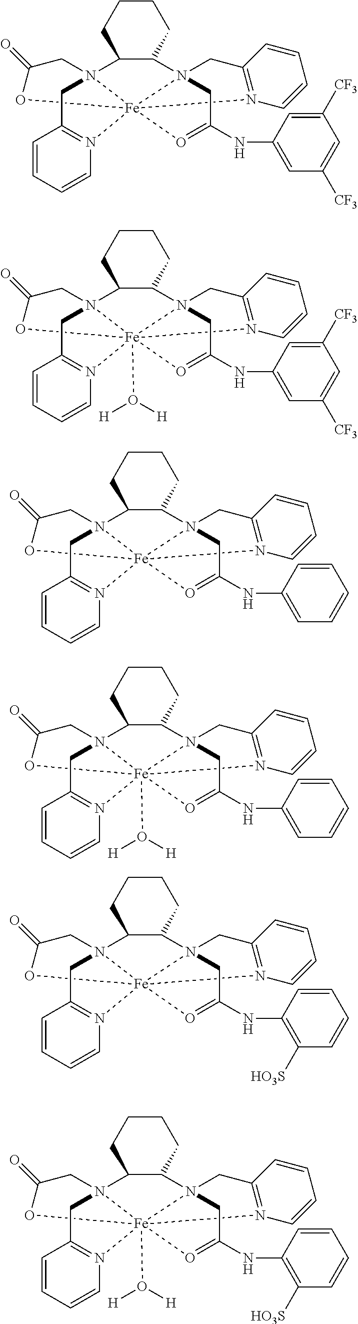

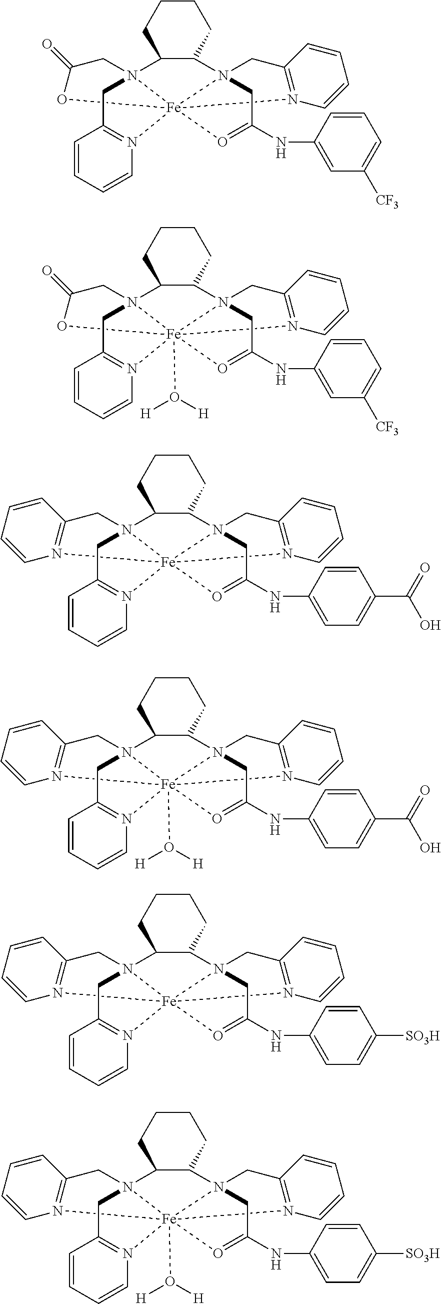

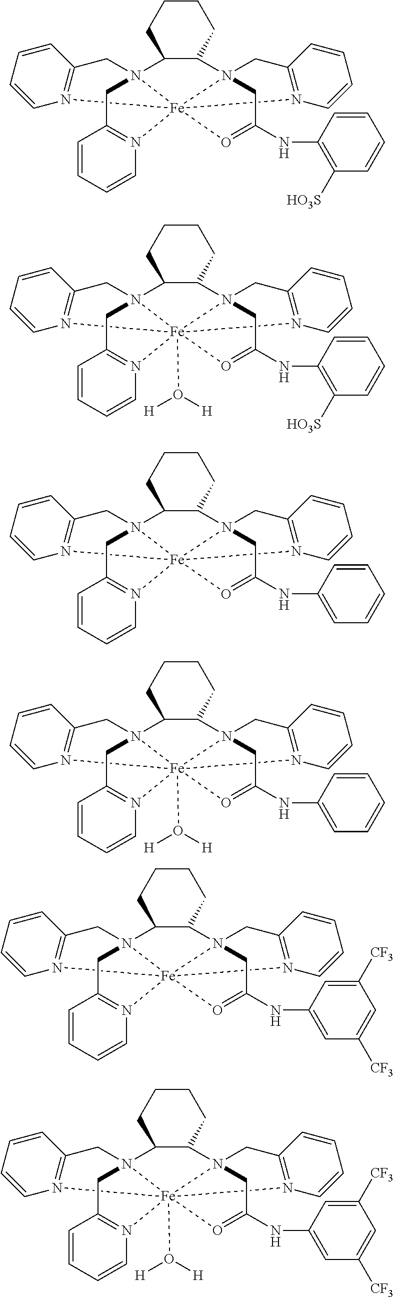

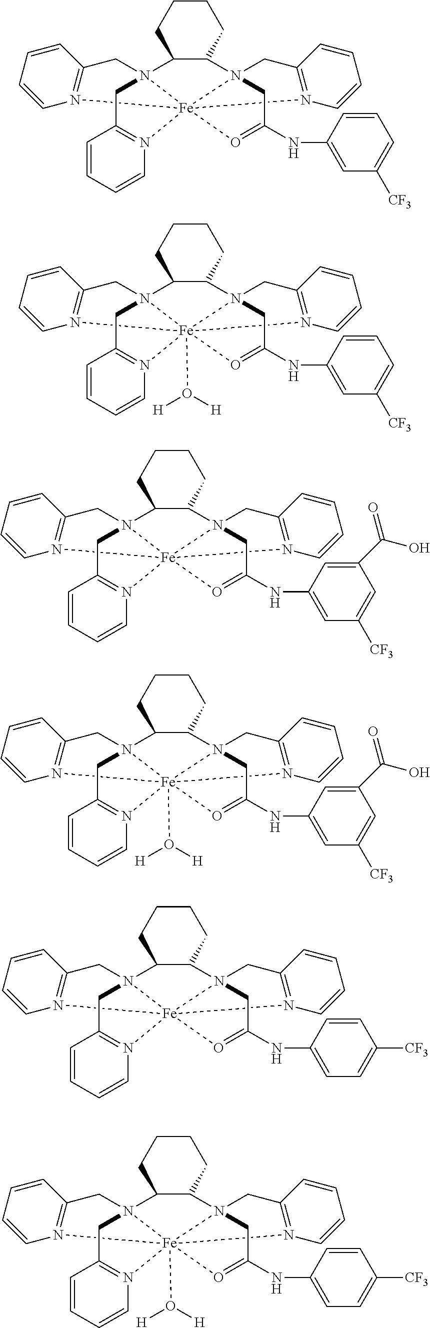

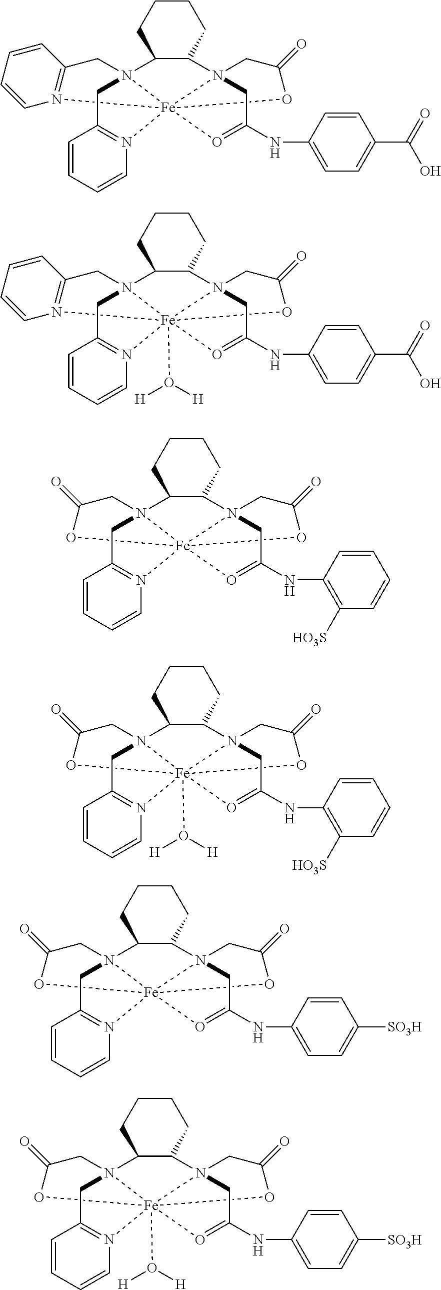

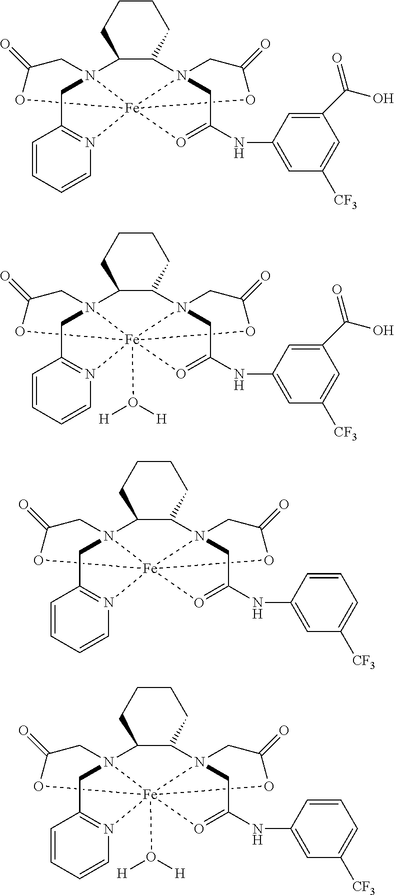

or a pharmaceutically acceptable salt thereof.

In some embodiments, the compound of Formula (A) has formula:

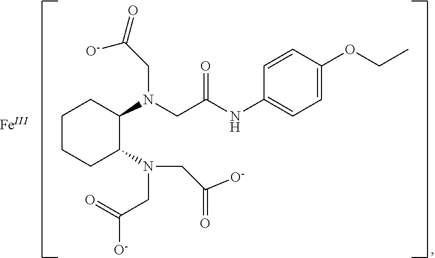

or a pharmaceutically acceptable salt thereof.

In some embodiments, the present disclosure provides a compound of Formula (B):

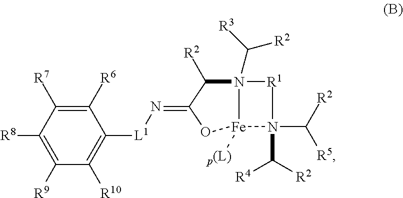

or a pharmaceutically acceptable salt thereof, wherein:
[symbol] indicates a coordinate bond;
each L is independently a Fe ligand;
p is 0, 1 or 2;
L¹ is C₁₋₃ alkylene; or L is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered (I)heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.

In some embodiments, Fe in the compound of Formula (B) is Fe (III). In some embodiments, Fe in the compound of Formula (A) is Fe (II).

In some embodiments, the compound of Formula (A) is not:

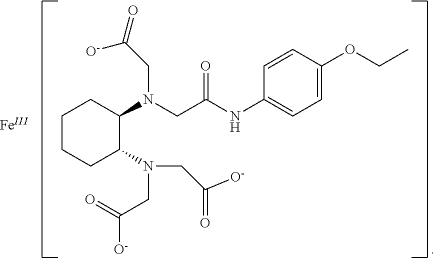

In some embodiments, L¹ is C₁₋₃ alkylene. In some embodiments, L¹ is methylene.

In some embodiments, L¹ is absent.

In some embodiments,

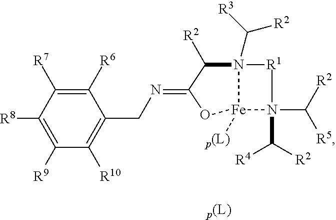

or a pharmaceutically acceptable salt thereof.

In some embodiments,

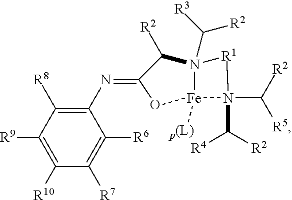

or a pharmaceutically acceptable salt thereof.

In some embodiments, R¹ is C₁₋₃ alkylene-ring A-C₁₋₃ alkylene, wherein said ring A is optionally substituted with 1, 2, or 3 substituents independently selected from R^{1A}.

In some embodiments, the compound has formula:

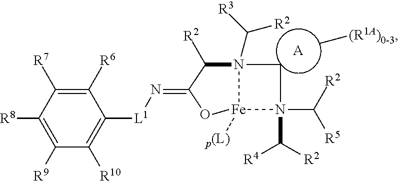

or a pharmaceutically acceptable salt thereof.

In some embodiments, ring A is phenylene, optionally substituted with R^{1A}.

In some embodiments, ring A is 5-6 membered heteroarylene, optionally substituted with R^{1A}.

In some embodiments, ring A is C₃-C₁₀ cycloalkylene, optionally substituted with R^{1A}. In some embodiments, ring A is cyclopentylene or cyclohexylene, optionally substituted with R^{1A}.

In some embodiments, ring A is a moiety of formula:

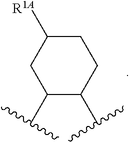

In some embodiments, ring A is 4-10 membered heterocycloalkylene, optionally substituted with R^{1A}.

In some embodiments, R¹ is C₂₋₆ alkylene, optionally substituted with R^{1A}.

In some embodiments, R¹ is 1,2-ethylene, optionally substituted with R^{1A}.

In some embodiments, R¹ is a moiety of formula:

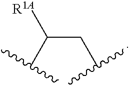

In some embodiments, each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, ring A is selected from any one of the following moieties:

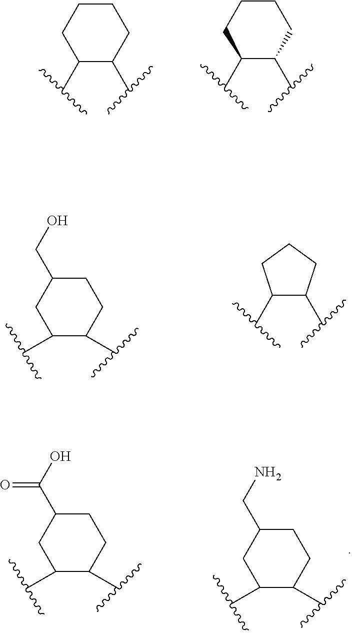 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

In some embodiments, ring A is selected from any one of the following moieties:

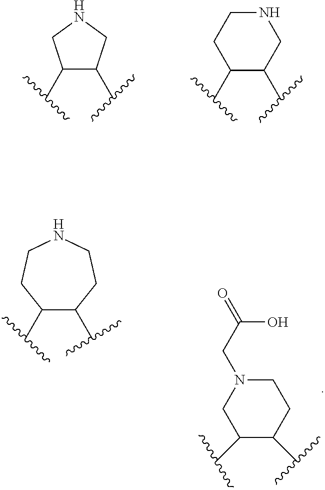 [SMILES: *C1CNCC1* ; *C1CCNCC1* ; *C1CCNCCC1* ; *C1CCN(CC(=O)O)CC1*]

In some embodiments, R¹ is selected from any one of the following moieties:

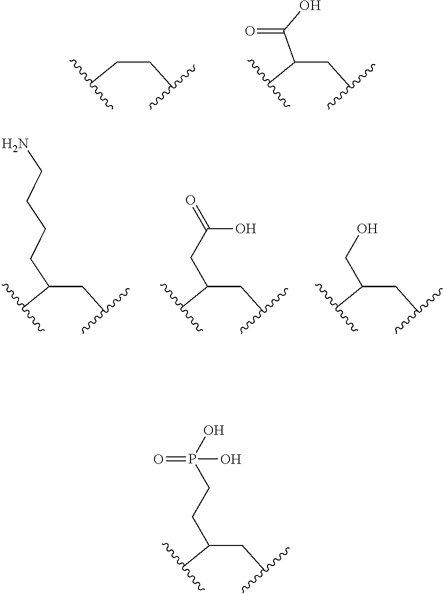

In some embodiments, R¹ is selected from the group consisting of a substituted or unsubstituted alkylene, such as 1,3-propylene or 1,2-ethylene, as shown below:

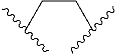 [SMILES: *CC*]

or 2,3-propylene-1-carboxylate, as shown below,

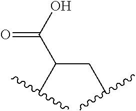 [SMILES: *CC(*)C(=O)O]

or 3,4-butylene-1-carboxylic acid, as shown below,

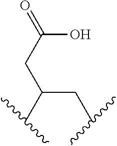 [SMILES: *CC(*)CC(=O)O]

or 1-hydroxy-3,4-butylene, as shown below,

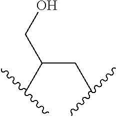 [SMILES: *CC(*)CO]

or 1-amino-5,6-hexylene, as shown below,

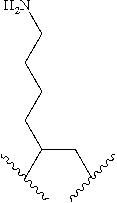 [SMILES: *CC(*)CCCCN]

substituted or unsubstituted cycloalkylene, such as those shown below, for example, cis- or trans-1,2-cyclohexylene, as shown below,

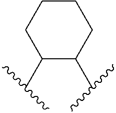 [SMILES: *C1CCCCC1*]

or trans-1,2-cyclohexylene, as shown below,

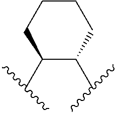 [SMILES: *[C@H]1CCCC[C@@H]1*]

or cis- or trans-1,2-cyclopentylene, as shown below,

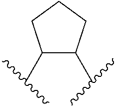 [SMILES: *C1CCCC1*]

substituted or unsubstituted monocyclic heterocyclyl, such as 2,5-dihydro-1H-pyrrolene, as shown below

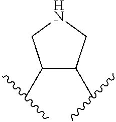 [SMILES: *C1CNCC1*]

1,2,3,6-tetrahydropyridinene, as shown below,

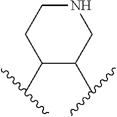 [SMILES: *C1CCNCC1*]

2,3,6,7-tetrahydro-1H-azepinene, as shown below,

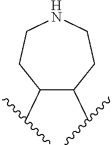 [SMILES: *C1CCNCCC1*]

In some embodiments, each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, each R² is H.

In some embodiments, at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, the compound has formula:

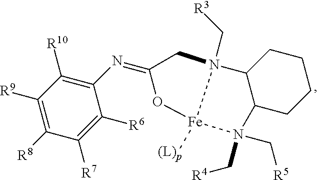

or a pharmaceutically acceptable salt thereof.

In some embodiments, the compound has formula:

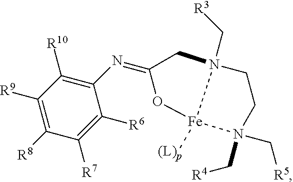

or a pharmaceutically acceptable salt thereof.

In some embodiments, R³ is ring B, optionally substituted with 1, 2, or 3 substituents independently selected from R^{1B}.

In some embodiments, R⁴ is ring C, optionally substituted with 1, 2, or 3 substituents independently selected from R^{1C}.

In some embodiments, R⁵ is ring D, optionally substituted with 1, 2, or 3 substituents independently selected from R^{1D}.

In some embodiments, ring B is 5-6 membered heteroaryl optionally substituted with 1, 2, or 3 substituents independently selected from R^{1B}.

In some embodiments, ring C is 5-6 membered heteroaryl optionally substituted with 1, 2, or 3 substituents independently selected from R^{1C}.

In some embodiments, ring D is 5-6 membered heteroaryl optionally substituted with 1, 2, or 3 substituents independently selected from R^{1D}.

In some embodiments, the 5-6 membered heteroaryl of ring B, ring C, or ring D is selected from any one of the following moieties:

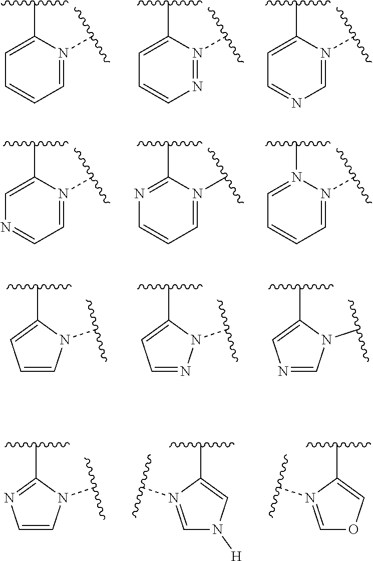

In some embodiments, ring B is 5-6 membered heterocycloalkyl, optionally substituted with 1, 2, or 3 substituents independently selected from R^{1B}.

In some embodiments, ring C is 5-6 membered heterocycloalkyl optionally substituted with 1, 2, or 3 substituents independently selected from R^{1C}.

In some embodiments, ring D is 5-6 membered heterocycloalkyl optionally substituted with 1, 2, or 3 substituents independently selected from R^{1D}.

In some embodiments, the 5-6 membered heterocycloalkyl of ring B, ring C, or ring D is selected from any one of the following moieties:

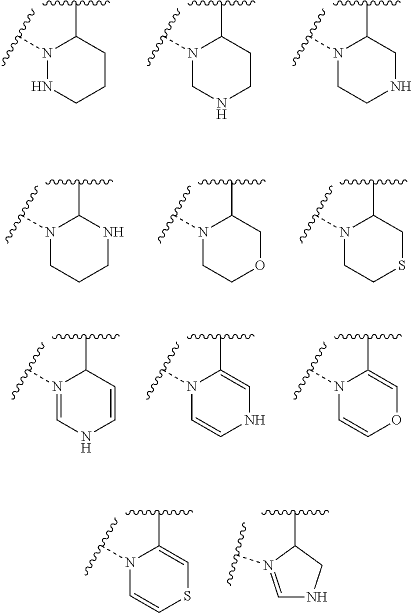

In some embodiments, each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1D} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, the 5-6 membered heteroaryl of ring B, ring C, or ring D is selected from any one of the following moieties:

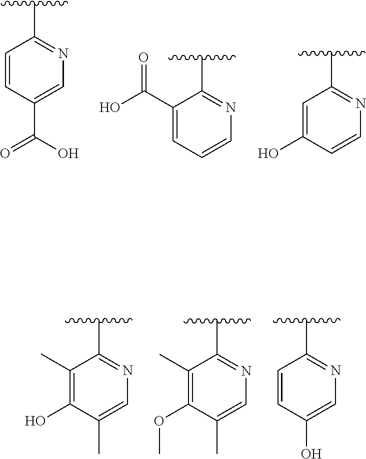

In some embodiments, R³ is C(O)O. In some embodiments, R³ is P(O)(O)(OR^{B}). In some embodiments, R³ is (C═O)NR^{B}.

In some embodiments, R⁴ is C(O)O. In some embodiments, R⁴ is P(O)(O)(OR^{B}). In some embodiments, R⁴ is (C═O)NR^{B}.

In some embodiments, the compound of Formula (B) has formula:

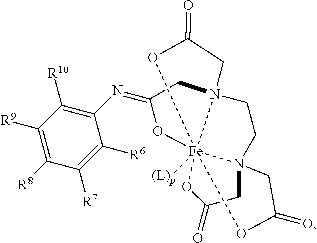

or a pharmaceutically acceptable salt thereof.

In some embodiments, the compound of Formula (B) has formula:

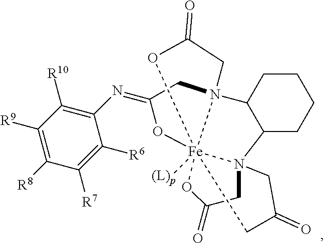

or a pharmaceutically acceptable salt thereof.

In some embodiments, R⁶, R⁷, R⁸, R⁹, and R¹⁰ are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, at least one of R⁶, R⁷, R⁸, R⁹, and R¹⁰ is selected from halo, C₁₋₆ haloalkyl, C₁₋₆ haloalkoxy.

In some embodiments, at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments:
R⁶, R⁸, and R¹⁰ are each H, and
R⁷ and R⁹ are each independently selected from halo, C₁₋₆ haloalkyl, and C₁₋₆ haloalkoxy.

In some embodiments, R⁷ and R⁹ are each independently selected from halo, C₁₋₆ haloalkyl, and C₁₋₆ haloalkoxy. In some embodiments, R⁷ and R⁹ are each independently C₁₋₆ haloalkyl.

In some embodiments, the compound of Formula (B) has formula:

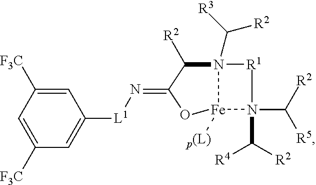

or a pharmaceutically acceptable salt thereof.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof. In some embodiments, p is 1 and L is H₂O.

In some embodiments, the compound of Formula (B) has formula:

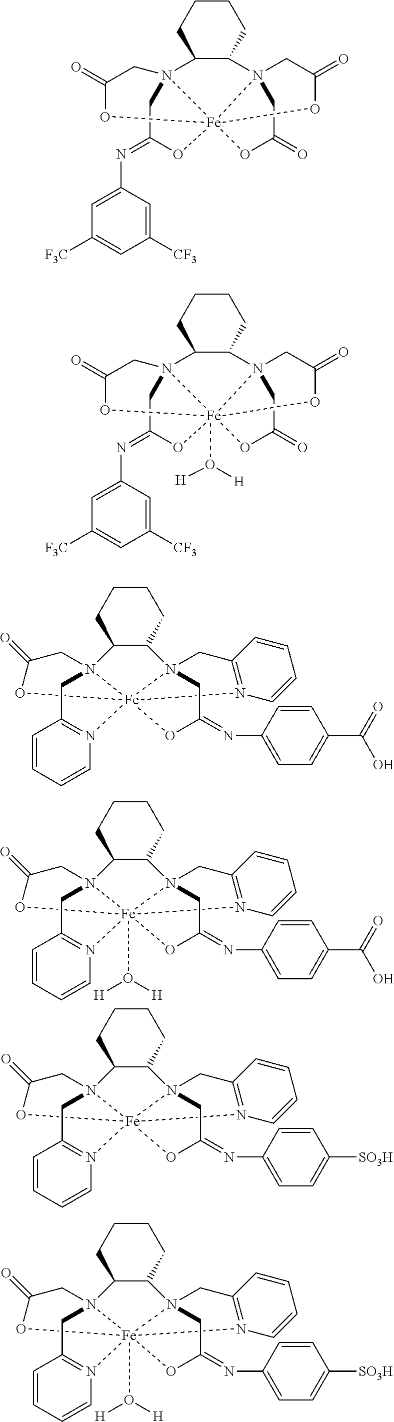

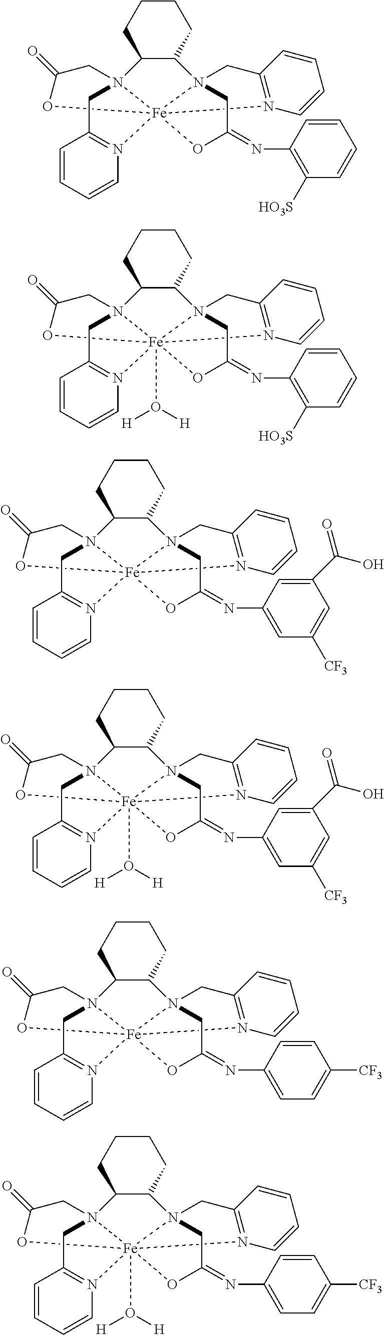

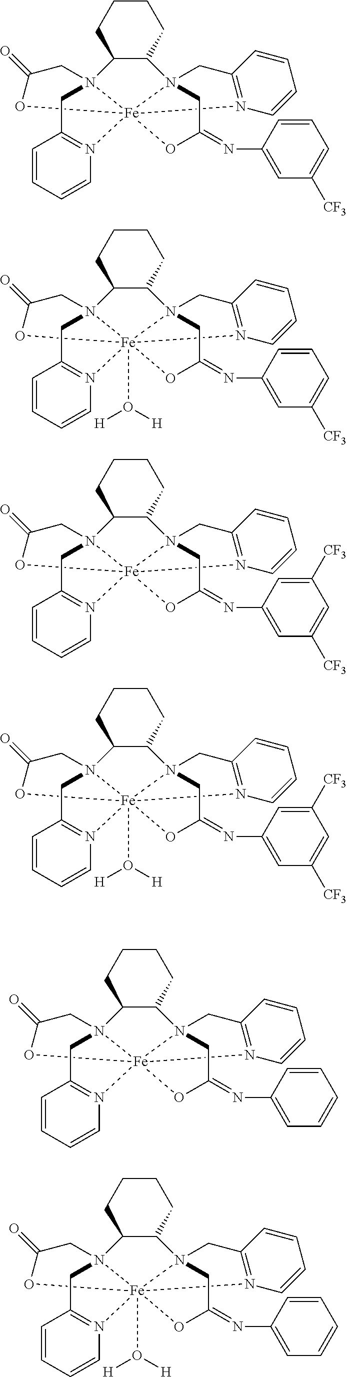

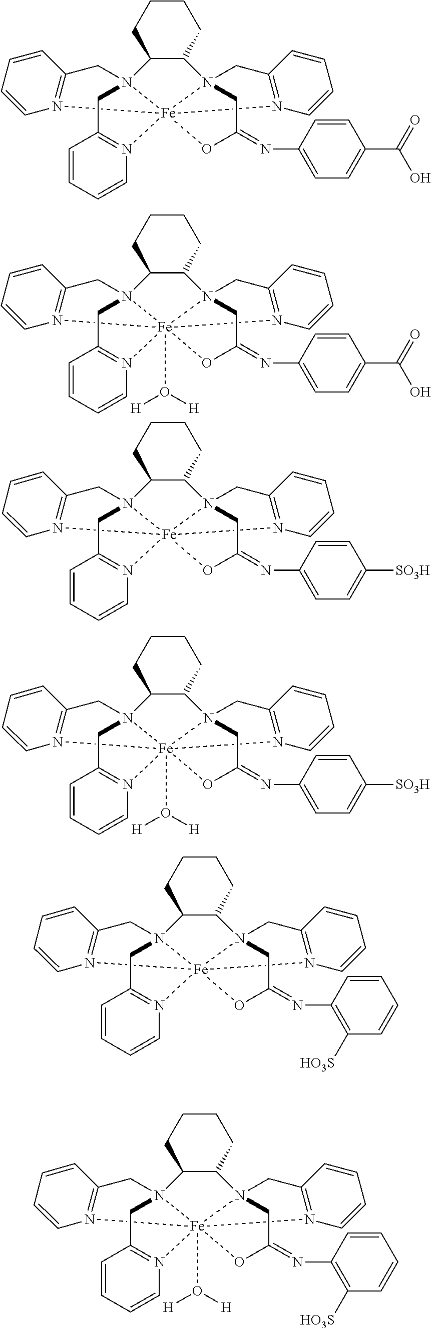

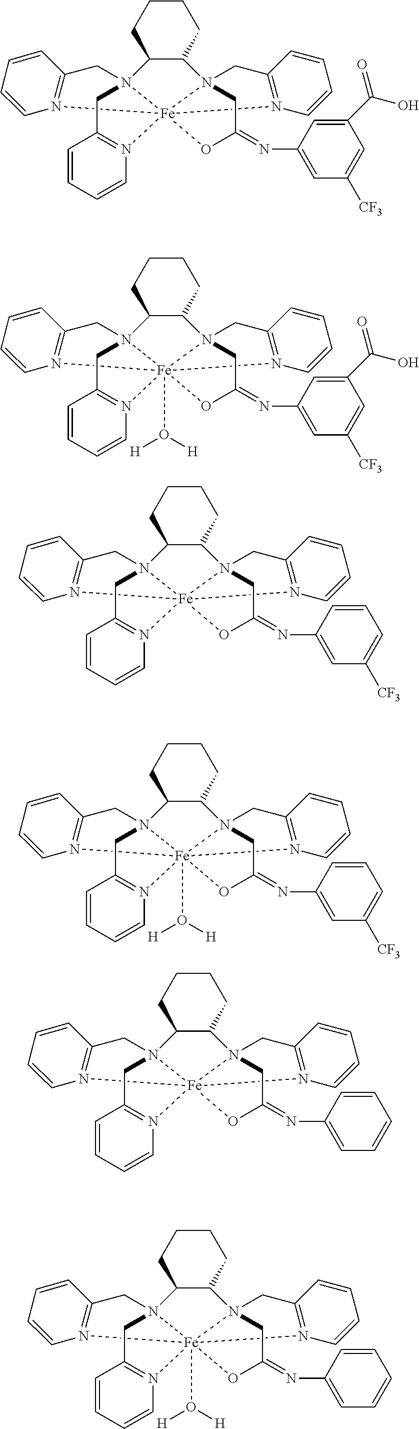

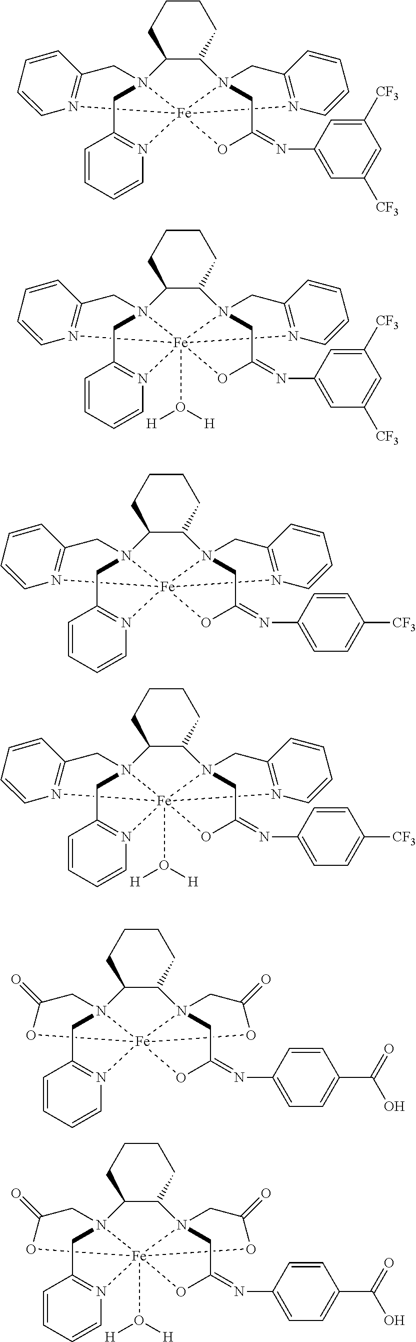

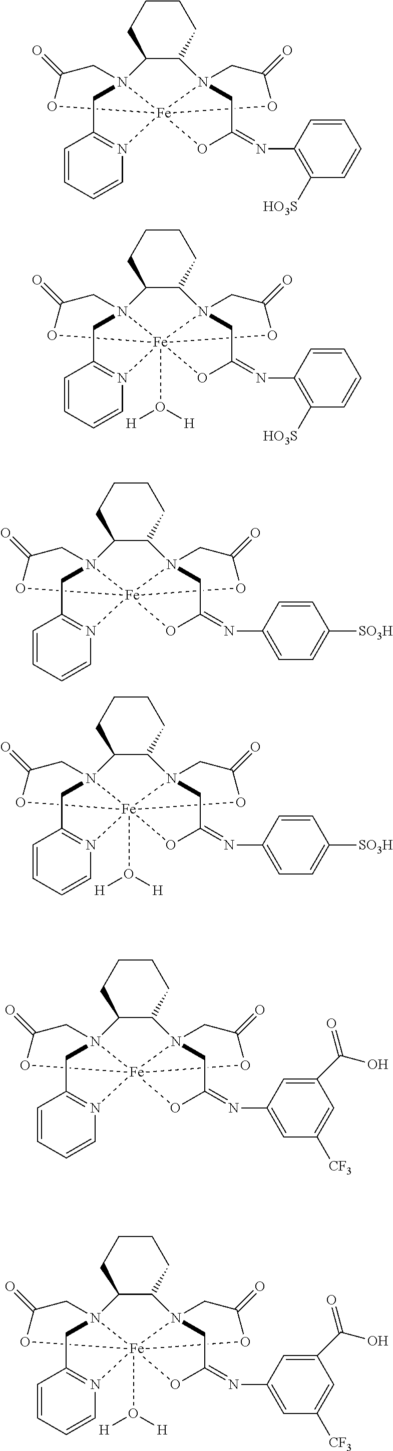

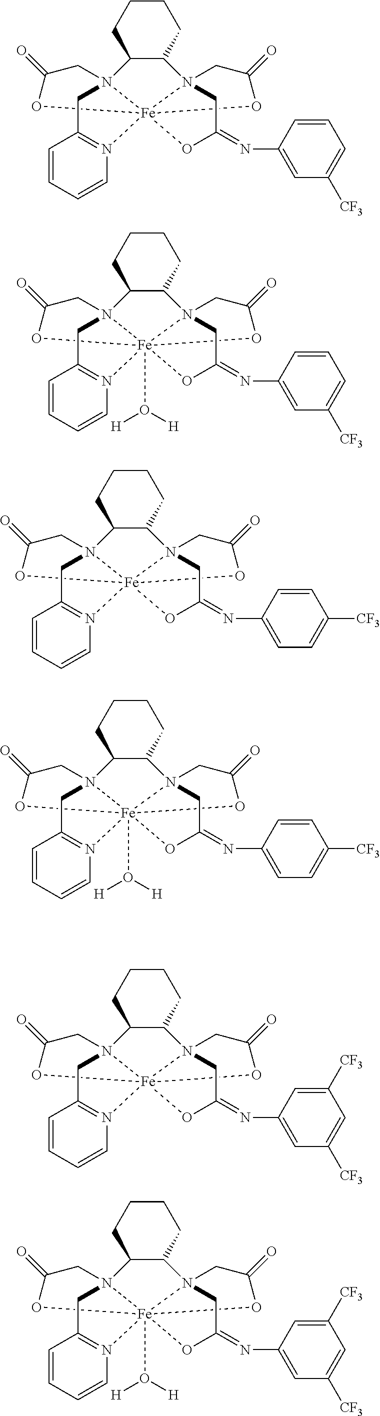

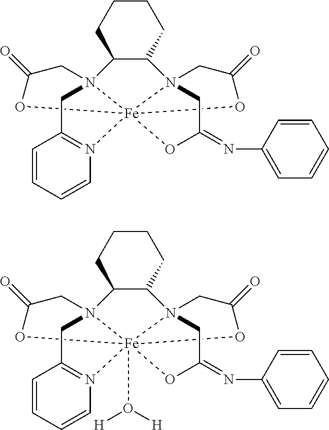

or a pharmaceutically acceptable salt thereof.

In some embodiments, the compound of Formula (A) or Formula (B) has a relaxivity of about 1.0 to about 5.0, for example, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, or any value in between. In some embodiments, the compound of Formula (A) or Formula (B) has a relaxivity of about 1.0 to about 3.0. In some embodiments, the compound of Formula (A) or Formula (B) has a relaxivity of about 1.0 to about 2.5. In some embodiments, the compound of Formula (A) or Formula (B) has a relaxivity of about 1.2 to about 2.3. In some embodiments, the compound of Formula (A) or Formula (B) has a relaxivity of about 1.3 to about 2.0. In some embodiments, the compound of Formula (A) or Formula (B) has a relaxivity of about 1.4 to about 1.9. In some embodiments, the compound of Formula (A) or Formula (B) has a relaxivity of about 1.4 or about 1.9.

### Pharmaceutically Acceptable Salts

In some embodiments, a salt of a compound of any of the Formulae is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁₋₆ alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds herein, or pharmaceutically acceptable salts thereof, are substantially isolated.

### Methods of Use

Positron emission tomography-magnetic resonance imaging (PET-MRI) is a hybrid imaging technology that incorporates magnetic resonance imaging (MRI) soft tissue morphological imaging and positron PET functional imaging. PET/MRI scans can be used to diagnose a health condition in humans and animals, e.g., for research and agricultural purposes. The compounds and compositions of this disclosure can be used as probes in PET/MRI, for example, for imaging organs and tissues, for diagnosing a disease, or for monitoring treatment or a disease.

For imaging a particular organ, such as the heart, a fusion approach is implemented using external fiducial landmarks provided by a “vest” optimized for the particular organ, e.g., for cardiac imaging. The vest surrounds the subject's chest to create a frame that follows minor movements due to transfer between scanners or light anesthesia. The tubes are filled with 15% iodine in water, rendering them visible in MRI. Subject motion is minimized with an imaging bed that can be used in both imaging systems. Exemplary MRI methods are set forth in U.S. patent application Ser. No. 09/778,585, entitled MAGNETIC RESONANCE ANGIOGRAPHY DATA, filed Feb. 7, 2001 and U.S. patent application Ser. No. 10/209,416, entitled SYSTEMS AND METHODS FOR TARGETED MAGNETIC RESONANCE IMAGING OF THE VASCULAR SYSTEM, filed Jul. 30, 2002, both of which are incorporated by reference in their entireties.

In some embodiments, the present disclosure provides a method of identifying and/or quantifying inflammatory processes in an organ or tissue of a subject. This may be attained, for example, by imaging the organ or tissue using MR imagining by administering a compound of Formula (A) to a subject. Without being bound by any theory, a compound of Formula (A) contains Fe in (II) oxidation state (Fe²), which is not by itself detected in the MR imaging. Upon reaching in inflamed area of the subject, the Fe²⁺ in the complex of Formula (A) simultaneously undergoes an oxidation (e.g., by ROS in the inflamed area) and deprotonation events thereby forming a stable complex of Formula (B) containing Fe in (III) oxidation state (Fe³), which is detectable by IRI. As such, administration of compound of Formula (A) to a subject advantageously allows for selective imaging of Formula (B) in the inflamed area of the organ or tissue.

In another general aspect, the present disclosure provides a method of magnetic resonance (MR) imaging of an organ or a tissue (e.g., organ or tissue affected by inflammation) of a subject, the method comprising:
i) administering to the subject an effective amount of a compound of Formula (A), or a pharmaceutically acceptable salt thereof, or a pharmaceutical composition comprising same;
ii) waiting a time sufficient to allow the compound of Formula (A) to accumulate in the organ or a tissue (e.g., organ or tissue affected by inflammation) to be imaged; and
ii) acquiring an MR image of the organ or the tissue (e.g., organ or tissue affected by inflammation) of the subject.

In some embodiments, the organ or the tissue comprises an area affected by acute, immune-mediated, autoimmune, or chronic inflammation. In some embodiments, the organ or the tissue is selected from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a cartilage, an epithelium, a tendon, and a ligament. In some embodiments, the organ is selected from lung, stomach, intestines, liver, thyroid, bladder, heart, eye, skin, kidney, gland, brain, pancreas, colon, lymph node, spleen, and prostate. In some embodiments, the tissue is epithelial tissue, mucosal tissue, connective tissue, muscle tissue, skin tissue, fibrous tissue, vascular tissue, or nervous tissue. In some embodiments, the imaging method comprises detecting blood clot, brain lesion, presence or absence of disrupted blood-brain-barrier, presence or absence of arterial stenosis, presence or absence of a solid tumor, or presence or absence of spinal stenosis, e.g., in the organ or tissue of the subject. In one example, the method allows imaging blood flow, clots, lesions, or the myocardium (e.g., inflamed areas of the myocardium) of the subject.

In some embodiments, administering comprises intravenous injection or infusion, e.g., in any of the dosage forms and in any amounts of the compound of Formula (A) described herein. In some embodiments, the sufficient time is about 5 minutes to about 120 minutes, for example, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes, or any value in between. In some embodiments, the sufficient time is about 5 minutes to about 45 minutes. In some embodiments, the sufficient time is about 30 minutes to about 60 minutes. In some embodiments, the sufficient time is about 45 minutes to about 90 minutes. In some embodiments, the sufficient time is about 60 minutes to about 120 minutes

In some embodiments, the present disclosure provides a method of diagnosing an inflammatory disease or condition in a subject, the method comprising:
i) administering to the subject an effective amount of a compound of Formula (A), or a pharmaceutically acceptable salt thereof, or a pharmaceutical composition comprising same;
ii) waiting a time sufficient (e.g., as described herein) to allow the compound of Formula (A) to accumulate in an organ or a tissue of the subject affected by the inflammatory disease or condition; and
iii) acquiring an MR image of the organ or the tissue of the subject,
wherein observing the MR image attributable to the compound of Formula (A) is indicative of the inflammatory disease or condition.

In yet another general aspect, the present disclosure provides a method of monitoring treatment of an inflammatory disease or condition in a subject, the method comprising:
i) administering to the subject an effective amount of a compound of Formula (A), or a pharmaceutically acceptable salt thereof, or a pharmaceutical composition comprising same;
ii) waiting a time sufficient to allow the compound to accumulate in an organ or tissue of the subject affected by the inflammatory disease or condition;
iii) acquiring a first MR image of the organ or the tissue of the subject;
iv) administering to the subject a therapeutic agent in an effective amount to treat the inflammatory disease or condition;
v) after iv), administering to the subject an effective amount of a compound of Formula (A), or a pharmaceutically acceptable salt thereof, or a pharmaceutical composition comprising same;
vi) waiting a time sufficient (e.g., as described herein) to allow the compound of Formula (A) to accumulate in the organ or tissue of the subject affected by the inflammatory disease or condition;
vii) acquiring a second MR image of the organ or the tissue of the subject; and
viii) comparing the first and the second MR images,
wherein observing a difference between the first and the second MR images attributable to the compound of Formula (A) is indicative of progression of treatment of inflammatory disease or condition.

In some embodiments, the inflammatory disease or condition is selected from cancer, kidney failure, diabetes, rheumatoid arthritis, osteoarthritis, inflammatory joint disease, lupus, tendinitis, colitis, ulcerative colitis, asthma, fatty liver disease, inflammatory diseases of the liver and gut, steatohepatitis, atherosclerosis, hyperlipidemia, dyslipidemia, rhinitis, conjunctivitis, oral inflammation, thyroid disease, lung disease, inflammatory bowel disease (IBD), Crohn's disease, chronic obstructive airways disease, multiple sclerosis (MS), chronic inflammatory demyelinating polyneuropathy, Guillain-Barre syndrome, psoriasis, Graves' disease, Hashimoto's disease, thyroiditis, myasthenia gravis, and vasculitis.

In some embodiments, the inflammatory disease or condition is kidney disease. In some embodiments, the inflammatory disease or condition is myocardial ischemia, acute coronary syndrome, myocardial infarction, stroke; nonalcoholic steatohepatitis; inflammatory bowel disease; multiple sclerosis; atherosclerotic plaque; tissue or organ ischemia; rheumatoid arthritis, metabolic disease, cardiovascular disease, or diabetes.

In some embodiments, the inflammatory disease is diabetes, rheumatoid arthritis, colitis, ulcerative colitis, asthma, allergic asthma, fatty liver disease, obesity, insulin resistance, inflammatory diseases of the liver and gut, steatohepatitis, liver inflammation, autoimmune disease, atherosclerosis, hyperlipidemia, dyslipidemia, rhinitis, conjunctivitis, oral inflammation, thyroid disease, lung disease, inflammatory bowel disease (IBD), Crohn's disease, or chronic obstructive airways disease.

Additional types of inflammatory disease or conditions include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis, Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, polymyalgia rheumatica, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, reperfusion injury, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, ulcerative colitis, undifferentiated connective tissue syndrome (UCTS), urticarial vasculitis, viral arthritis, Wegener's granulomatosis, Whipple's disease, Wilson's disease, and yersinial arthritis.

Suitable examples of cardiovascular diseases include hypertension, atherosclerosis, thrombosis, deep vein thrombosis, pulmonary embolism, coronary artery disease (CAD), ischemia/reperfusion injury, ischemia, cerebral ischemia, heart attack, stroke, myocardial infarction, angina, heart failure, hypertensive heart disease, rheumatic heart disease, cardiomyopathy, celiac disease, cardiac arrest, high blood pressure, arrhythmia, congenital heart disease, peripheral artery disease, restenosis, ischemic stroke, and bronchial asthma.

Suitable examples of cancer include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. In some embodiments, the cancer is prostate cancer. In some embodiments, cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the subject can be monitored for effectiveness of treatment with a steroid or non-steroid anti-inflammatory agent, e.g., ibuprofen, naproxen, indomethacin, diclofenac, aspirin, loxoprofen, and the like. In some embodiments, the subject can be monitored for treatment of cancer with a chemotherapeutic agents such as antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine. Other examples of anti-cancer agents include alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide. Other examples of anti-cancer agents include kinase inhibitors such as imatinib, sorafenib, sunitinib, and vemurafenib. Other examples of anti-cancer agents include cancer immunotherapy such as ipilimumab, pembrolizumab, nivolumab, atezolizumab, bevacizumab, trastuzumab, and cetuximab. In some embodiments, the compound of the present disclosure can be used to monitor and/or assess effectiveness of a radiotherapy.

In some embodiments, the present disclosure provides a method of magnetic resonance (MR) imaging of an organ or a tissue (e.g., any of the organs or tissues described herein) of a subject, the method comprising:
i) administering to the subject an effective amount of a compound of Formula B (e.g., comprising Fe³⁺ ion), or a pharmaceutically acceptable salt thereof, or a pharmaceutical composition comprising same;
ii) waiting a time sufficient (e.g., as described herein) to allow the compound to accumulate in the organ or a tissue to be imaged; and
ii) acquiring an MR image of the organ or the tissue of the subject.

In some embodiments, a method of diagnosing a disease or condition (e.g., any of the conditions described herein) in a subject, the method comprising:
i) administering to the subject an effective amount of a compound of Formula (B), or a pharmaceutically acceptable salt thereof, or a pharmaceutical composition comprising same;
ii) waiting a time sufficient (e.g., as described herein) to allow the compound to accumulate in an organ or a tissue of the subject affected by the disease or condition; and
iii) acquiring an MR image of the organ or the tissue of the subject,
wherein observing the MR image attributable to the compound of Formula (B) is indicative of the disease or condition.

In some embodiments, the present disclosure provides a method of monitoring treatment of a disease or condition (e.g., any disease as described herein) in a subject, the method comprising:
i) administering to the subject an effective amount of a compound of Formula (B), or a pharmaceutically acceptable salt thereof, or a pharmaceutical composition comprising same;
ii) waiting a time sufficient to allow the compound of Formula (B) to accumulate in an organ or tissue of the subject affected by the disease or condition;
iii) acquiring a first MR image of the organ or the tissue of the subject;
iv) administering to the subject a therapeutic agent in an effective amount to treat the disease or condition;
v) after iv), administering to the subject an effective amount of a compound of Formula (B), or a pharmaceutically acceptable salt thereof, or a pharmaceutical composition comprising same;
vi) waiting a time sufficient to allow the compound of Formula (B) to accumulate in the organ or tissue of the subject affected by the disease or condition;
vii) acquiring a second MR image of the organ or the tissue of the subject; and
viii) comparing the first and the second MR images,
wherein observing a difference between the first and the second MR images attributable to the compound of Formula (B) is indicative of progression of treatment of the disease or condition.

In some embodiments, the method may also comprise comparing images obtained from subjects exhibiting the symptoms of the disease or condition with the images obtained from healthy subjects. In one example, overabundance of images attributable to compound of Formula (A) or (B) in the brain of the subject (as compared to healthy subject) may be indicative of a neurodegenerative disease such as Alzheimer's disease or a related condition. In some embodiments, the disease or condition is selected from cancer, blood clot, brain lesion, disrupted blood-brain-barrier, arterial stenosis, and spinal stenosis.

### Compositions, Formulations, and Routes of Administration

The present application also provides pharmaceutical compositions comprising an effective amount of a compound of the present disclosure (e.g., an iron complex of Formula A or B, or a pharmaceutically acceptable salt thereof) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

### Dosages and Regimens

In the pharmaceutical compositions of the present application, a compound of the present disclosure (e.g., an iron complex of a compound of Formula A or B) is present in an effective amount (e.g., a therapeutically effective amount). Effective doses may vary, depending on the organ imaged, the disease being diagnosed, the treatment being monitored, as well as the sex, age and general health condition of the subject, excipient usage, the possibility of interaction with therapeutic treatments such as use of other agents, and the judgment of the treating physician, or the imaging technician, or a diagnostician.

In some embodiments, an effective amount of the compound (e.g., an iron complex of a Formula A or B) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound such as an iron complex of Formula A or B is about 0.1 mg/kg, about 0.5 mg/kg, about 1 mg/kg, about 2 mg/kg, or about 5 mg/kg.

In some embodiments, an effective amount of the compound (e.g., an iron complex of formula A or B) can range, for example, from about 1 μg Fe/kg to about 500 μg Fe/kg, from about 2 μg Fe/kg to about 300 μg Fe, from about 5 μg Fe/kg to about 250 μg Fe/kg/kg, from about 1 μg Fe/kg to about 50 μg Fe/kg, from about 5 μg Fe/kg to about 100 μg Fe/kg/kg, from about 5 μg Fe/kg to about 50 μg Fe/kg/kg, from about 1 μg Fe/kg to about 25 μg Fe/kg/kg, or from about 1 μg Fe/kg to about 10 μg Fe/kg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). The frequency of administration can be determined on the particular imaging needs.

### Definitions

As used herein, the term “about” means “approximately” (e.g., plus or minus approximately 10% of the indicated value).

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, the term “NC—C₁₋₃ alkyl” refers to a group of formula —(C₁₋₃ alkylene)-CN.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. The term “arylene” refers to a divalent aryl group.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl. The term “cycloalkylene” refers to a divalent cycloalkyl group.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl. The term “heteroarylene” refers to a divalent heteroaryl group.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. The term “heterocycloalkylene” refers to a divalent heterocycloalkyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

The terms “chelating ligand,” “chelating moiety,” and “chelate moiety” are used interchangeably and refer to any polydentate ligand that is capable of coordinating a metal ion, either directly or after removal of protecting groups, or is a reagent, with or without suitable protecting groups, that is used in the synthesis of a MR contrast agent and comprises substantially all of the atoms that ultimately will coordinate the metal ion of the final metal complex. The terms “chelate” or “metal chelate” refer to the actual metal-ligand complex. It is understood that the polydentate ligand can eventually be coordinated to a medically useful or diagnostic metal ion.

Coordination of metal ions by water and other ligands is often regarded in terms of coordination spheres (see e.g., D. T. Richens, The Chemistry of Aqua Ions, John Wiley and Sons, New York, 1997, Chapter 1). The first or primary coordination sphere represents all the ligands directly bonded to the metal ion and is defined by the ligands. There is a second coordination sphere where water molecules and counterions bond to the groups in the first coordination sphere via hydrogen bonding and electrostatic interactions. Tertiary and subsequent coordination spheres are typically termed “bulk water” or “bulk solvent”. The distinctions between these spheres are both spatial and temporal. The first coordination sphere is typically well-defined and the time that a water or other ligand spends in the first coordination sphere is longer than in other coordination spheres. The second sphere is less well-defined, but the waters here have a longer lifetime than the typical diffusion time of water. Beyond the second sphere water diffuses freely.

The term “specific binding affinity” as used herein, refers to the capacity of a contrast agent to be taken up by, retained by, or bound to a particular or target biological component to a greater degree as compared to other non-targeted biological components. Contrast agents that have this property are said to be “targeted” to the “target” component. Contrast agents that lack this property are said to be “non-specific” or “non-targeted” agents. The binding affinity of a binding group for a target is expressed in terms of the equilibrium dissociation constant “K_{d}.”

The term “relaxivity” as used herein, refers to the increase in either of the MR quantities 1/T₁ or 1/T₂ per millimolar (mM) concentration of paramagnetic ion or contrast agent, which quantities may be different if the contrast agent contains a multiplicity of paramagnetic ions, wherein T₁ is the longitudinal or spin-lattice relaxation time, and T₂ is the transverse or spin-spin relaxation time of water protons or other imaging or spectroscopic nuclei, including protons found in molecules other than water. Relaxivity is expressed in units of mM⁻¹s⁻¹.

### EMBODIMENTS

1. Embodiment 1 provide a compound of Formula (A):

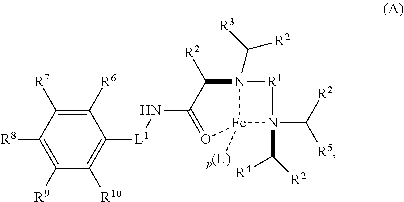

or a pharmaceutically acceptable salt thereof, wherein:
[symbol] indicates a coordinate bond;
each L is independently a Fe ligand;
p is 0, 1 or 2;
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}SO₂NR^{A}R^{B}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃RAB, and —(C₁₋₆ alkyl)_{q}(C═O)NHSO₂R^{A};
each q is independently 0 or 1; and
each R^{A} and R^{B} are independently hydrogen or C₁₋₆ alkyl;
with a proviso that the compound of Formula (A) is not:

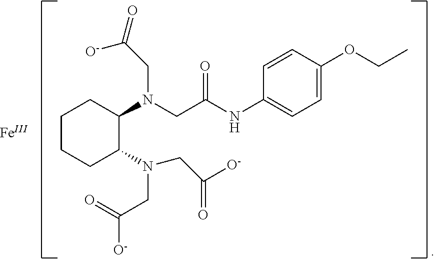

2. The compound of embodiment 1, wherein L¹ is C₁₋₃ alkylene.
3. The compound of embodiment 2, wherein L¹ is methylene.
4. The compound of embodiment 1, wherein L¹ is absent.
5. The compound of any one of embodiments 1-4, wherein R¹ is C₁₋₃ alkylene-ring A-C₁₋₃ alkylene, wherein said ring A is optionally substituted with 1, 2, or 3 substituents independently selected from R^{1A}.
6. The compound of any one of embodiments 1-4, wherein the compound has formula:

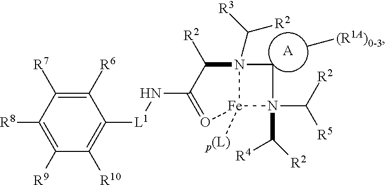

or a pharmaceutically acceptable salt thereof.
7. The compound of embodiment 6, wherein ring A is phenylene, optionally substituted with R^{1A}.
8. The compound of embodiment 6, wherein ring A is 5-6 membered heteroarylene, optionally substituted with R^{1A}.
9. The compound of embodiment 6, wherein ring A is C₃-C₁₀ cycloalkylene, optionally substituted with R^{1A}.
10. The compound of embodiment 9, wherein ring A is cyclopentylene or cyclohexylene, optionally substituted with R^{1A}.
11. The compound of embodiment 10, wherein ring A is a moiety of formula:

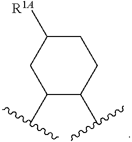

12. The compound of embodiment 6, wherein ring A is 4-10 membered heterocycloalkylene, optionally substituted with R^{1A}.
13. The compound of any one of embodiments 1-4, wherein R¹ is C₂₋₆ alkylene, optionally substituted with R^{1A}.
14. The compound of embodiment 13, wherein R¹ is 1,2-ethylene, optionally substituted with R^{1A}.
15. The compound of embodiment 14, wherein R¹ is a moiety of formula:

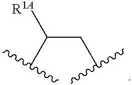

16. The compound of any one of embodiments 1-15, wherein each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
17. The compound of embodiment 10, wherein ring A is selected from any one of the following moieties:

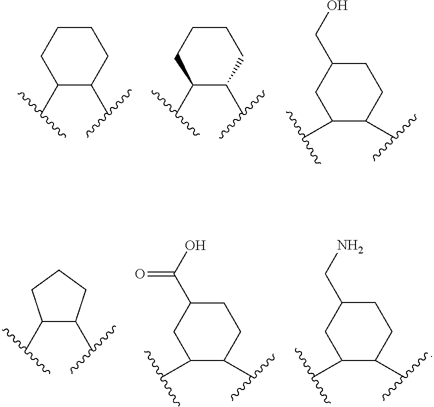 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

18. The compound of embodiment 12, wherein ring A is selected from any one of the following moieties:

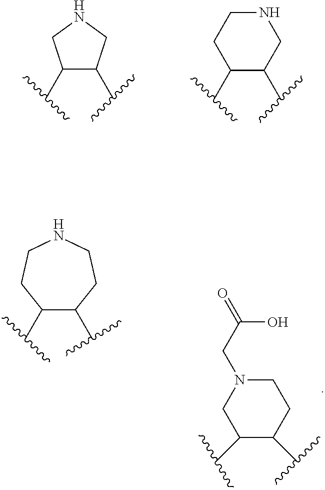 [SMILES: *C1CNCC1* ; *C1CCNCC1* ; *C1CCNCCC1* ; *C1CCN(CC(=O)O)CC1*]

19. The compound of embodiment 15, wherein R¹ is selected from any one of the following moieties:

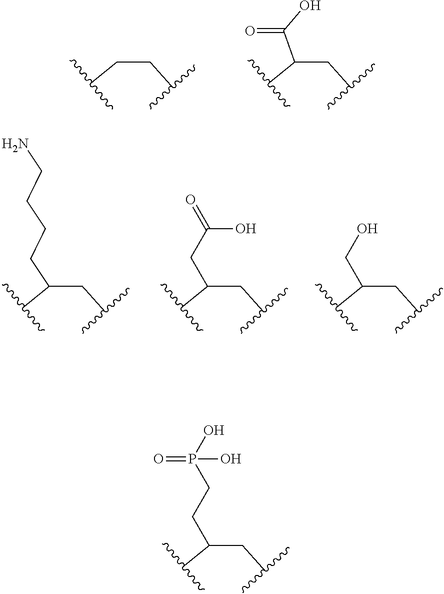

20. The compound of any one of embodiments 1-19, wherein each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
21. The compound of embodiment 20, wherein each R² is H.
22. The compound of embodiment 20, wherein at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
23. The compound of any one of embodiments 1-6, wherein the compound has formula:

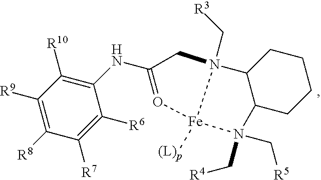

or a pharmaceutically acceptable salt thereof.
24. The compound of any one of embodiments 1-6, wherein the compound has formula:

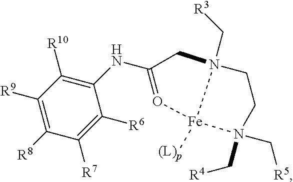

or a pharmaceutically acceptable salt thereof.
25. The compound of any one of embodiments 1-24, wherein R³ is ring B, optionally substituted with 1, 2, or 3 substituents independently selected from R^{1B}.
26. The compound of any one of embodiments 1-25, wherein R⁴ is ring C, optionally substituted with 1, 2, or 3 substituents independently selected from R^{1C}.
27. The compound of any one of embodiments 1-26, wherein R⁵ is ring D, optionally substituted with 1, 2, or 3 substituents independently selected from R^{1D}.
28. The compound of embodiment 25, wherein ring B is 5-6 membered heteroaryl optionally substituted with 1, 2, or 3 substituents independently selected from R^{1B}.
29. The compound of embodiment 26, wherein ring C is 5-6 membered heteroaryl optionally substituted with 1, 2, or 3 substituents independently selected from R^{1C}.
30. The compound of embodiment 27, wherein ring D is 5-6 membered heteroaryl optionally substituted with 1, 2, or 3 substituents independently selected from R^{1D}.
31. The compound of any one of embodiments 28-30, wherein the 5-6 membered heteroaryl is selected from any one of the following moieties:

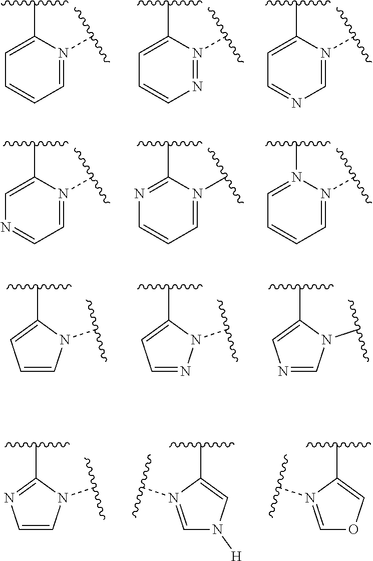

32. The compound of embodiment 25, wherein ring B is 5-6 membered heterocycloalkyl, optionally substituted with 1, 2, or 3 substituents independently selected from R^{1B}.
33. The compound of embodiment 26, wherein ring C is 5-6 membered heterocycloalkyl optionally substituted with 1, 2, or 3 substituents independently selected from R^{1C}.
34. The compound of embodiment 27, wherein ring D is 5-6 membered heterocycloalkyl optionally substituted with 1, 2, or 3 substituents independently selected from R^{1D}.
35. The compound of any one of embodiments 32-34, wherein the 5-6 membered heterocycloalkyl is selected from any one of the following moieties:

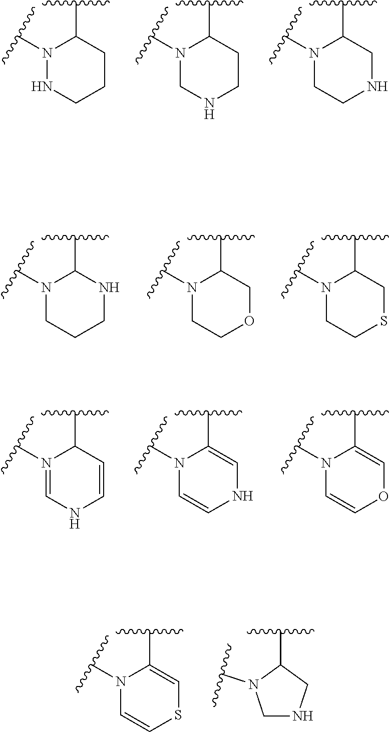

36. The compound of any one of embodiments 1-35, wherein each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
37. The compound of any one of embodiments 1-36, wherein each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
38. The compound of any one of embodiments 1-37, wherein each R^{1D} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
39. The compound of any one of embodiments 1-24, wherein R³ is C(O)O.
40. The compound of any one of embodiments 1-24, wherein R⁴ is C(O)O.
41. The compound of any one of embodiments 1-24, wherein R⁵ is C(O)O.
42. The compound of embodiment 24, having formula:

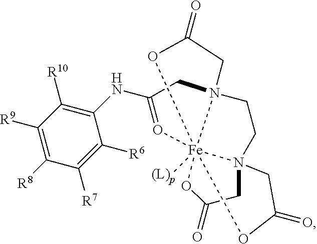

or a pharmaceutically acceptable salt thereof.
43. The compound of embodiment 23, having formula:

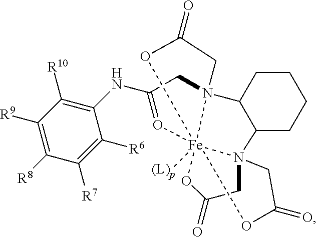

or a pharmaceutically acceptable salt thereof.
44. The compound of any one of embodiments 1-43, wherein R⁶, R⁷, R⁸, R⁹, and R¹⁰ are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
45. The compound of any one of embodiments 1-43, wherein at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
46. The compound of any one of embodiments 1-43, wherein at least one of R⁶, R⁷, R⁸, R⁹, and R¹⁰ is selected from halo, C₁₋₆ haloalkyl, C₁₋₆ haloalkoxy.
47. The compound of any one of embodiments 1-43, wherein at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
48. The compound of any one of embodiments 1-43, wherein:
   R⁶, R⁸, and R¹⁰ are each H, and
   R⁷ and R⁹ are each independently selected from halo, C₁₋₆ haloalkyl, and C₁₋₆ haloalkoxy.
49. The compound of any one of embodiments 1-48, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.
50. The compound of embodiment 1, selected from any one of the following compounds:

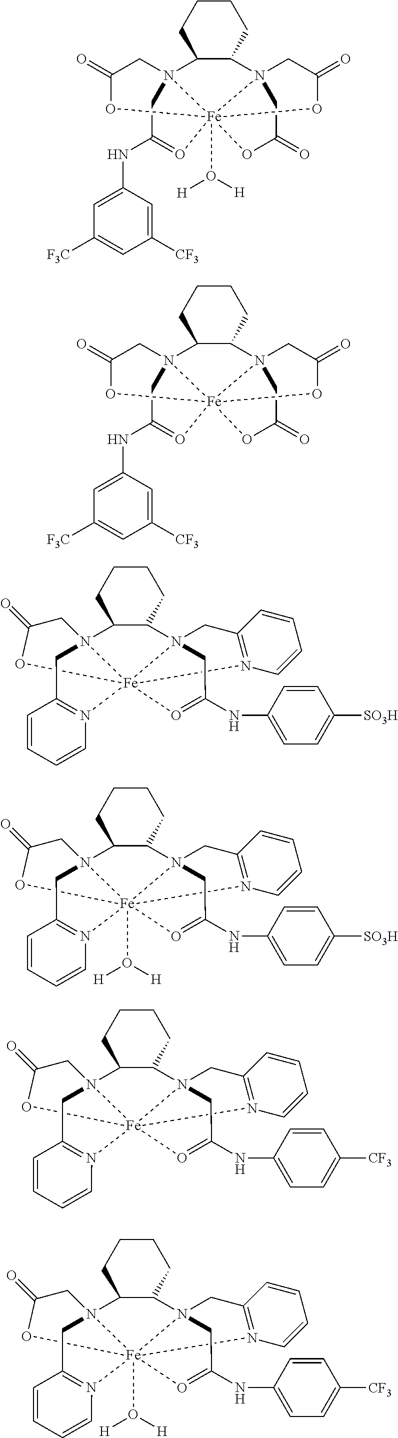

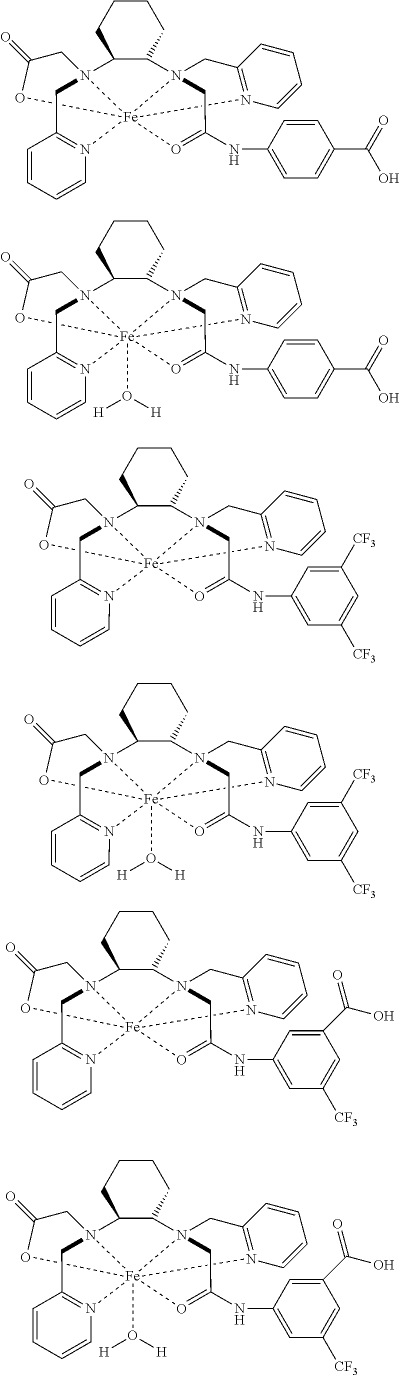

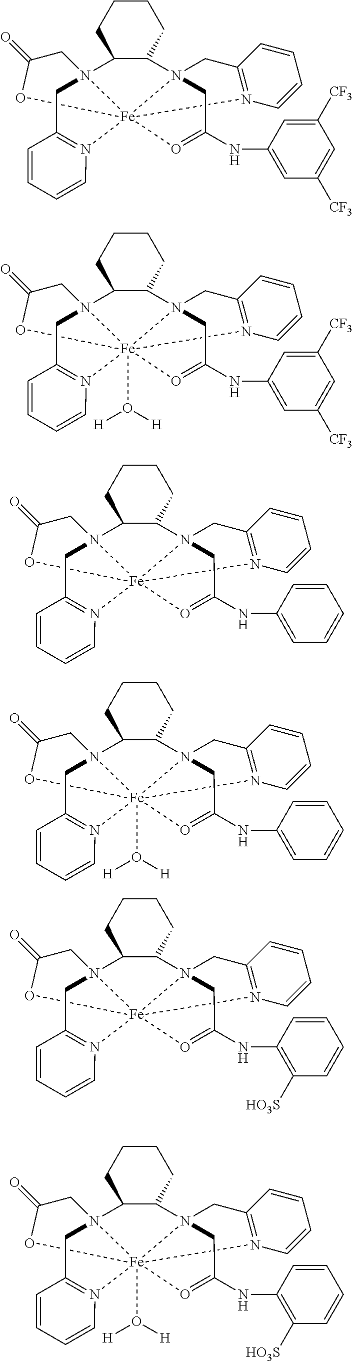

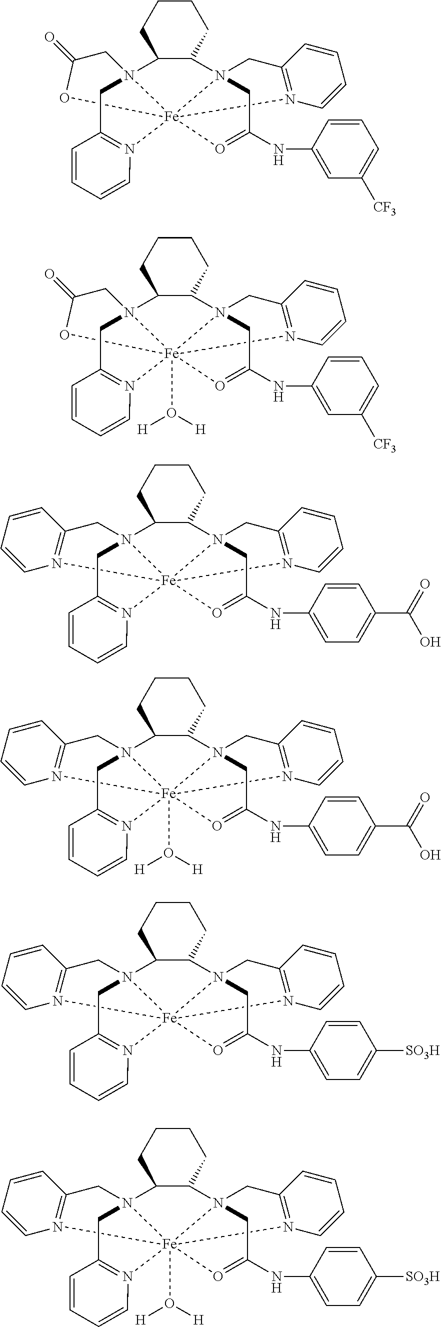

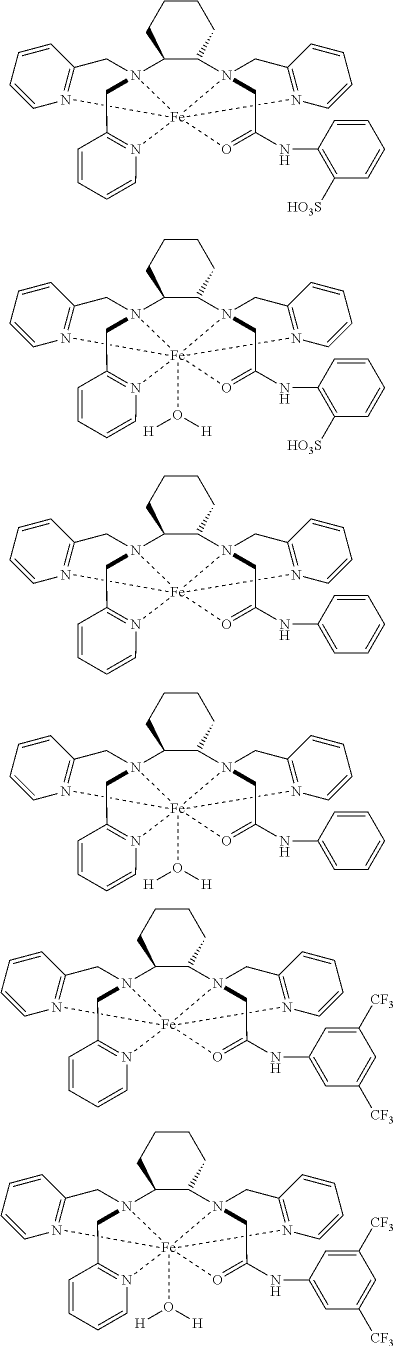

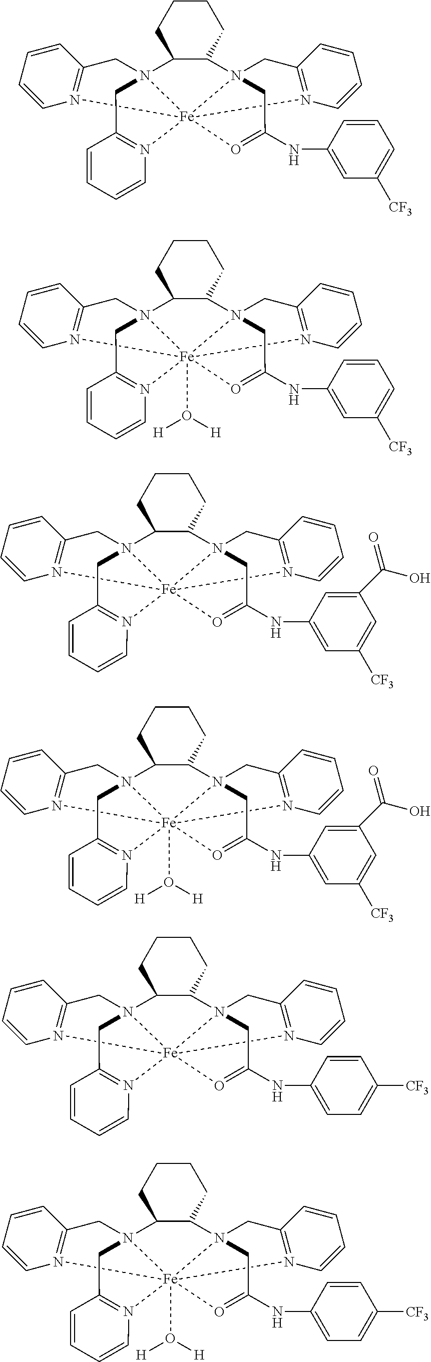

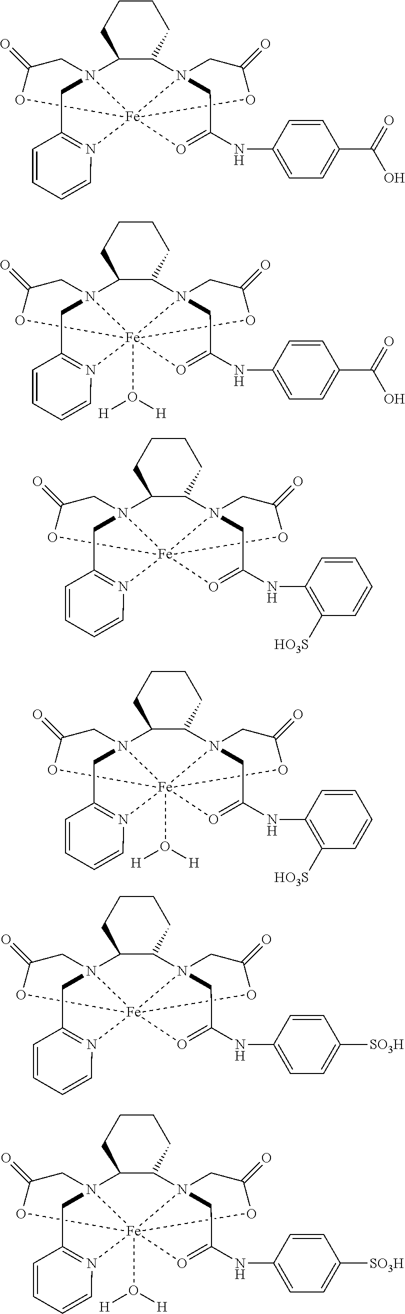

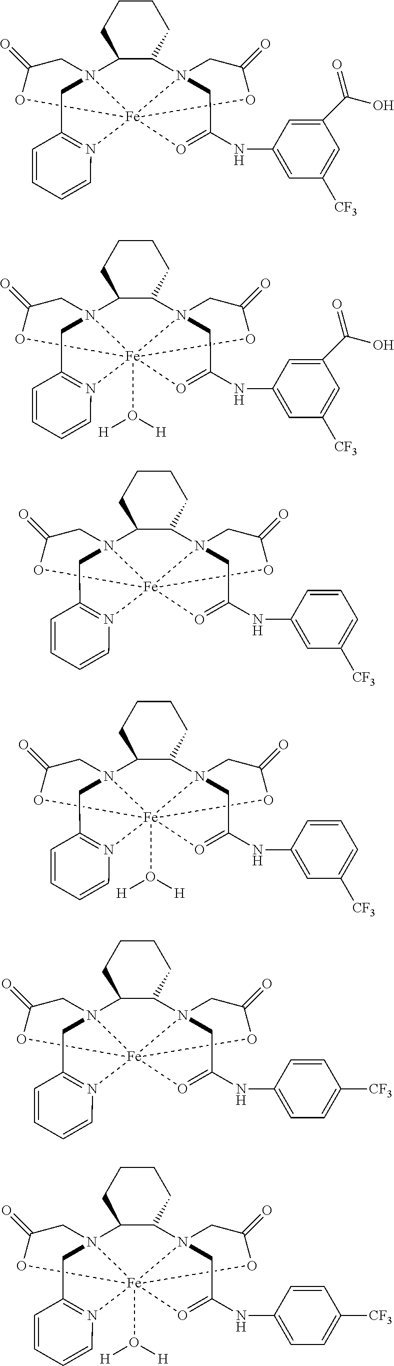

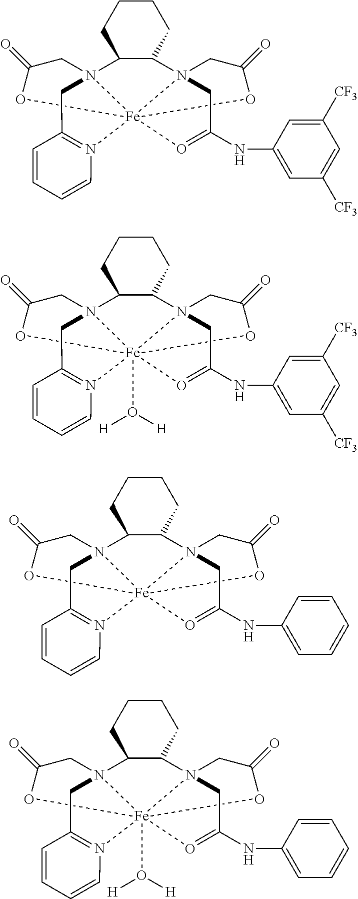

or a pharmaceutically acceptable salt thereof.
51. A pharmaceutical composition comprising a compound of any one of embodiments 1-50, or a pharmaceutically acceptable salt thereof, and a pharmaceutically acceptable carrier.
52. A method of magnetic resonance (MR) imaging of an organ or a tissue of a subject, the method comprising:
   i) administering to the subject an effective amount of a compound of any one of embodiments 1-50, or a pharmaceutically acceptable salt thereof, or a pharmaceutical composition of embodiment 51;
   ii) waiting a time sufficient to allow the compound to accumulate in the organ or a tissue to be imaged; and
   ii) acquiring an MR image of the organ or the tissue of the subject.
53. The method of embodiment 52, wherein the organ or the tissue comprises an area affected by acute or chronic inflammation.
54. The method of embodiment 52 or embodiment 53, wherein the organ or the tissue is selected from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a cartilage, an epithelium, a tendon, and a ligament.
55. The method of embodiment 52, comprising detecting blood clot, brain lesion, presence or absence of disrupted blood-brain-barrier, presence or absence of arterial stenosis, presence or absence of a solid tumor, or presence or absence of spinal stenosis.
56. A method of diagnosing an inflammatory disease or condition in a subject, the method comprising:
   i) administering to the subject an effective amount of a compound of any one of embodiments 1-50, or a pharmaceutically acceptable salt thereof, or a pharmaceutical composition of embodiment 51;
   ii) waiting a time sufficient to allow the compound to accumulate in an organ or a tissue of the subject affected by the inflammatory disease or condition; and
   iii) acquiring an MR image of the organ or the tissue of the subject, wherein observing the MR image attributable to the compound of Formula (A) is indicative of the inflammatory disease or condition.
57. A method of monitoring treatment of an inflammatory disease or condition in a subject, the method comprising:
   i) administering to the subject an effective amount of a compound of any one of embodiments 1-50, or a pharmaceutically acceptable salt thereof, or a pharmaceutical composition of embodiment 51;
   ii) waiting a time sufficient to allow the compound to accumulate in an organ or tissue of the subject affected by the inflammatory disease or condition;
   iii) acquiring a first MR image of the organ or the tissue of the subject;
   iv) administering to the subject a therapeutic agent in an effective amount to treat the inflammatory disease or condition;
   v) after iv), administering to the subject an effective amount of a compound of any one of embodiments 1-50, or a pharmaceutically acceptable salt thereof, or a pharmaceutical composition of embodiment 51
   vi) waiting a time sufficient to allow the compound to accumulate in the organ or tissue of the subject affected by the inflammatory disease or condition;
   vii) acquiring a second MR image of the organ or the tissue of the subject; and
   viii) comparing the first and the second MR images,
   wherein observing a difference between the first and the second MR images attributable to the compound of Formula (A) is indicative of progression of treatment of inflammatory disease or condition.
58. The method of embodiment 56 or embodiment 57, wherein the inflammatory disease or condition is selected from cancer, kidney failure, diabetes, rheumatoid arthritis, osteoarthritis, inflammatory joint disease, lupus, tendinitis, colitis, ulcerative colitis, asthma, fatty liver disease, inflammatory diseases of the liver and gut, steatohepatitis, atherosclerosis, hyperlipidemia, dyslipidemia, rhinitis, conjunctivitis, oral inflammation, thyroid disease, lung disease, inflammatory bowel disease (IBD), Crohn's disease, chronic obstructive airways disease, multiple sclerosis (MS), chronic inflammatory demyelinating polyneuropathy, Guillain-Barre syndrome, psoriasis, Graves' disease, Hashimoto's disease, thyroiditis, myasthenia gravis, and vasculitis.
59. A compound of Formula (B):

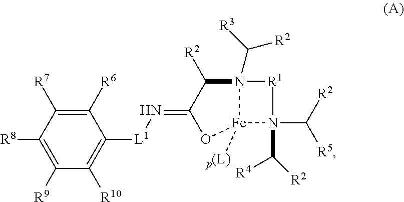

or a pharmaceutically acceptable salt thereof, wherein:
[symbol] indicates a coordinate bond;
each L is independently a Fe ligand;
p is 0, 1 or 2;
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₆ alkyl)_{q}SO₂NR^{A}R^{B}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.
60. The compound of embodiment 59, wherein L¹ is C₁₋₃ alkylene.
61. The compound of embodiment 60, wherein L¹ is methylene.
62. The compound of embodiment 60, wherein L¹ is absent.
63. The compound of any one of embodiments 59-62, wherein R¹ is C₁₋₃ alkylene-ring A-C₁₋₃ alkylene, wherein said ring A is optionally substituted with 1, 2, or 3 substituents independently selected from R^{1A}.
64. The compound of any one of embodiments 59-62, wherein the compound has formula:

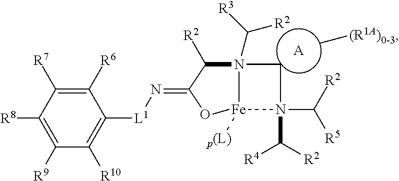

or a pharmaceutically acceptable salt thereof.
65. The compound of embodiment 64, wherein ring A is phenylene, optionally substituted with R^{1A}.
66. The compound of embodiment 64, wherein ring A is 5-6 membered heteroarylene, optionally substituted with R^{1A}.
67. The compound of embodiment 64, wherein ring A is C₃-C₁₀ cycloalkylene, optionally substituted with R^{1A}.
68. The compound of embodiment 67, wherein ring A is cyclopentylene or cyclohexylene, optionally substituted with R^{1A}.
69. The compound of embodiment 68, wherein ring A is a moiety of formula:

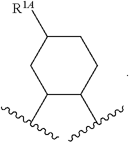

70. The compound of embodiment 64, wherein ring A is 4-10 membered heterocycloalkylene, optionally substituted with R^{1A}.
71. The compound of any one of embodiments 59-62, wherein R¹ is C₂₋₆ alkylene, optionally substituted with R^{1A}.
72. The compound of embodiment 71, wherein R¹ is 1,2-ethylene, optionally substituted with R^{1A}.
73. The compound of embodiment 72, wherein R¹ is a moiety of formula:

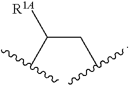

74. The compound of any one of embodiments 59-73, wherein each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
75. The compound of embodiment 69, wherein ring A is selected from any one of the following moieties:

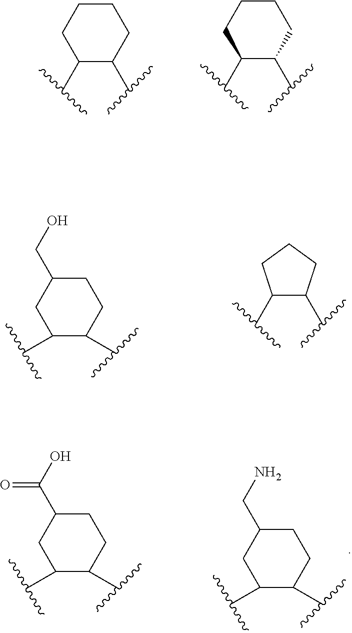 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

76. The compound of embodiment 70, wherein ring A is selected from any one of the following moieties:

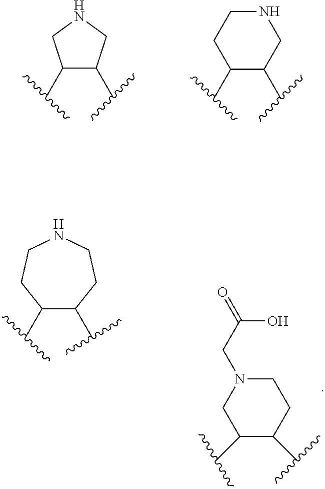 [SMILES: *C1CNCC1* ; *C1CCNCC1* ; *C1CCNCCC1* ; *C1CCN(CC(=O)O)CC1*]

77. The compound of embodiment 72, wherein R¹ is selected from any one of the following moieties:

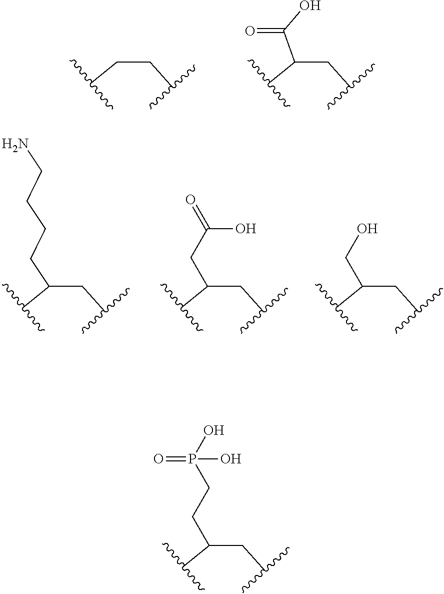

78. The compound of any one of embodiments 59-77, wherein each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
79. The compound of embodiment 78, wherein each R² is H.
80. The compound of embodiment 78, wherein at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
81. The compound of any one of embodiments 59-64, wherein the compound has formula:

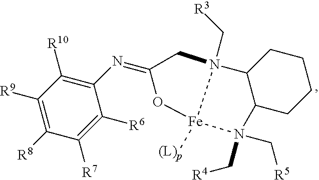

or a pharmaceutically acceptable salt thereof.
82. The compound of any one of embodiments 59-62, wherein the compound has formula:

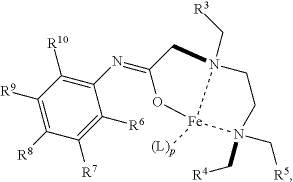

or a pharmaceutically acceptable salt thereof.
83. The compound of any one of embodiments 59-82, wherein R³ is ring B, optionally substituted with 1, 2, or 3 substituents independently selected from R^{1B}.
84. The compound of any one of embodiments 59-83, wherein R⁴ is ring C, optionally substituted with 1, 2, or 3 substituents independently selected from R^{1C}.
85. The compound of any one of embodiments 59-84, wherein R⁵ is ring D, optionally substituted with 1, 2, or 3 substituents independently selected from R^{1D}.
86. The compound of embodiment 83, wherein ring B is 5-6 membered heteroaryl optionally substituted with 1, 2, or 3 substituents independently selected from R^{B}.
87. The compound of embodiment 84, wherein ring C is 5-6 membered heteroaryl optionally substituted with 1, 2, or 3 substituents independently selected from R^{1C}.
88. The compound of embodiment 85, wherein ring D is 5-6 membered heteroaryl optionally substituted with 1, 2, or 3 substituents independently selected from R^{1D}.
89. The compound of any one of embodiments 86-88, wherein the 5-6 membered heteroaryl is selected from any one of the following moieties:

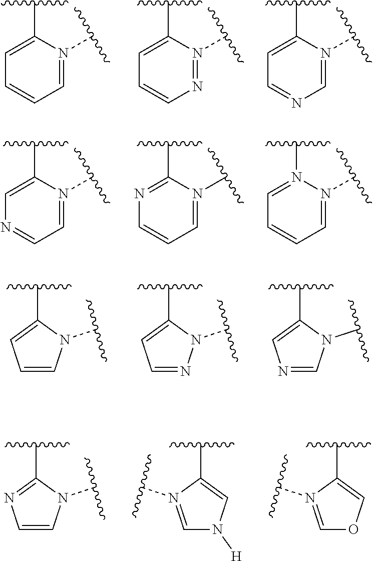

90. The compound of embodiment 83, wherein ring B is 5-6 membered heterocycloalkyl, optionally substituted with 1, 2, or 3 substituents independently selected from R^{1B}.
91. The compound of embodiment 84, wherein ring C is 5-6 membered heterocycloalkyl optionally substituted with 1, 2, or 3 substituents independently selected from R^{1C}.
92. The compound of embodiment 85, wherein ring D is 5-6 membered heterocycloalkyl optionally substituted with 1, 2, or 3 substituents independently selected from R^{1D}.
93. The compound of any one of embodiments 90-92, wherein the 5-6 membered heterocycloalkyl is selected from any one of the following moieties:

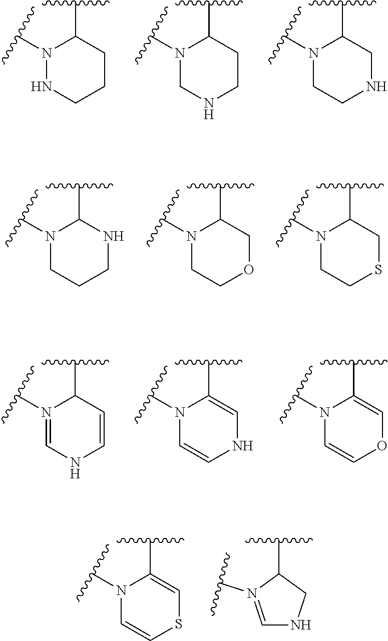

94. The compound of any one of embodiments 59-93, wherein each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
95. The compound of any one of embodiments 59-94, wherein each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
96. The compound of any one of embodiments 59-95, wherein each R^{1D} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
97. The compound of any one of embodiments 59-82, wherein R³ is C(O)O.
98. The compound of any one of embodiments 59-82, wherein R⁴ is C(O)O.
99. The compound of any one of embodiments 59-82, wherein R⁵ is C(O)O.
100. The compound of embodiment 81, having formula:

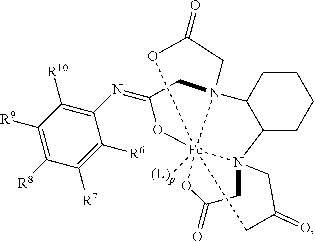

or a pharmaceutically acceptable salt thereof.
101. The compound of embodiment 82 having formula:

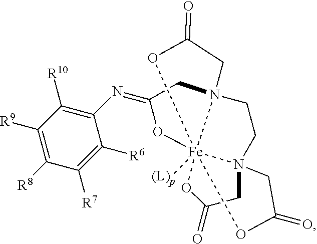

or a pharmaceutically acceptable salt thereof.
102. The compound of any one of embodiments 59-101, wherein R⁶, R⁷, R⁸, R⁹, and R10 are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
103. The compound of any one of embodiments 59-101, wherein at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
104. The compound of any one of embodiments 59-101, wherein at least one of R⁶, R⁷, R⁸, R⁹, and R¹⁰ is selected from halo, C₁₋₆ haloalkyl, C₁₋₆ haloalkoxy.
105. The compound of any one of embodiments 59-101, wherein at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
106. The compound of any one of embodiments 59-101, wherein:
   R⁶, R⁸, and R¹⁰ are each H, and
   R⁷ and R⁹ are each independently selected from halo, C₁₋₆ haloalkyl, and C₁₋₆ haloalkoxy.
107. The compound of any one of embodiments 59-106, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.
108. The compound of embodiment 59, selected from any one of the following compounds:

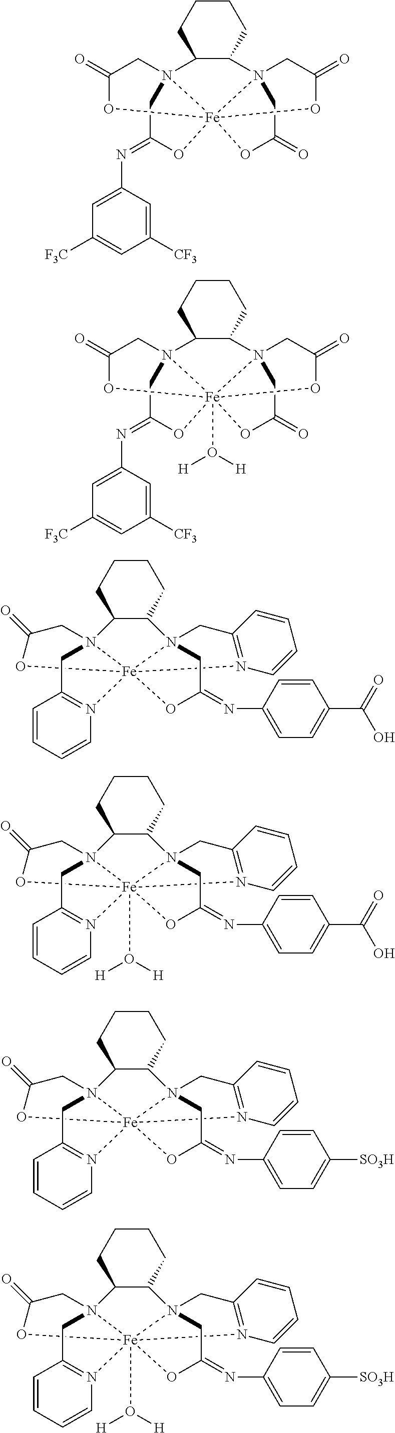

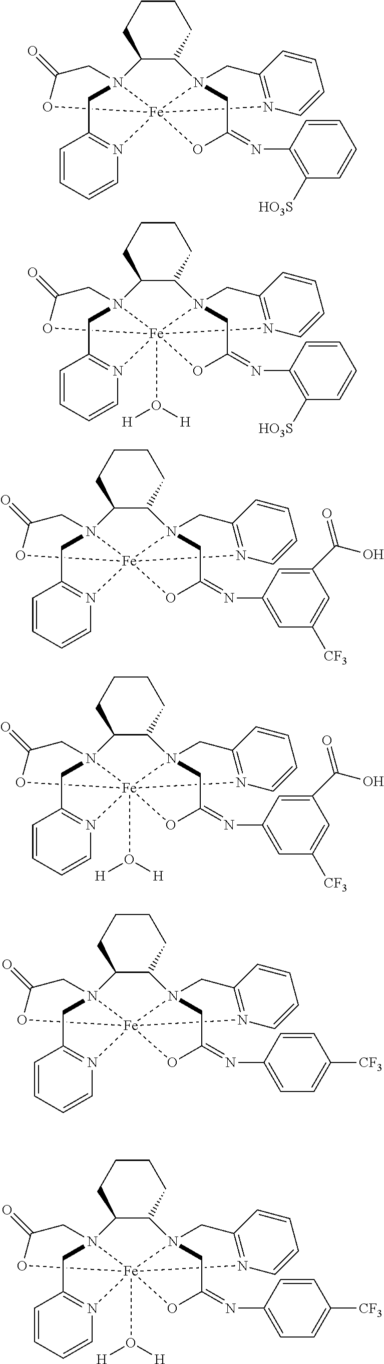

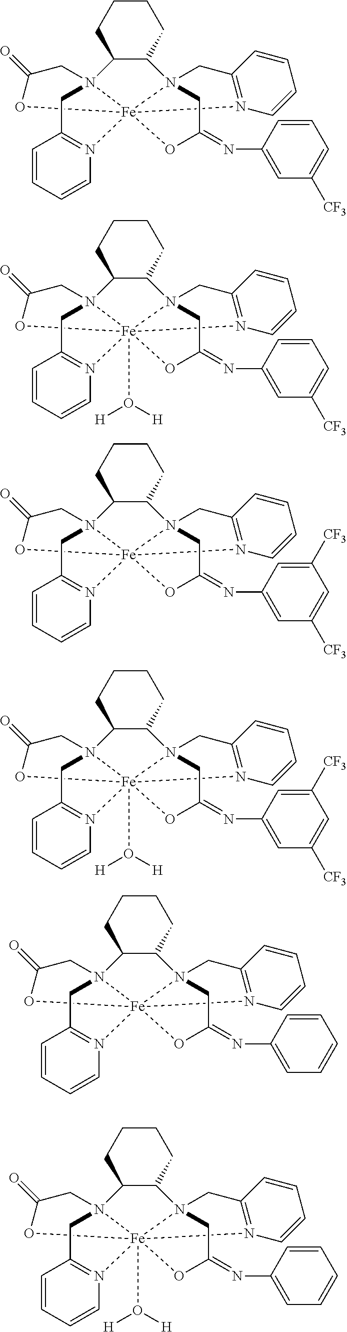

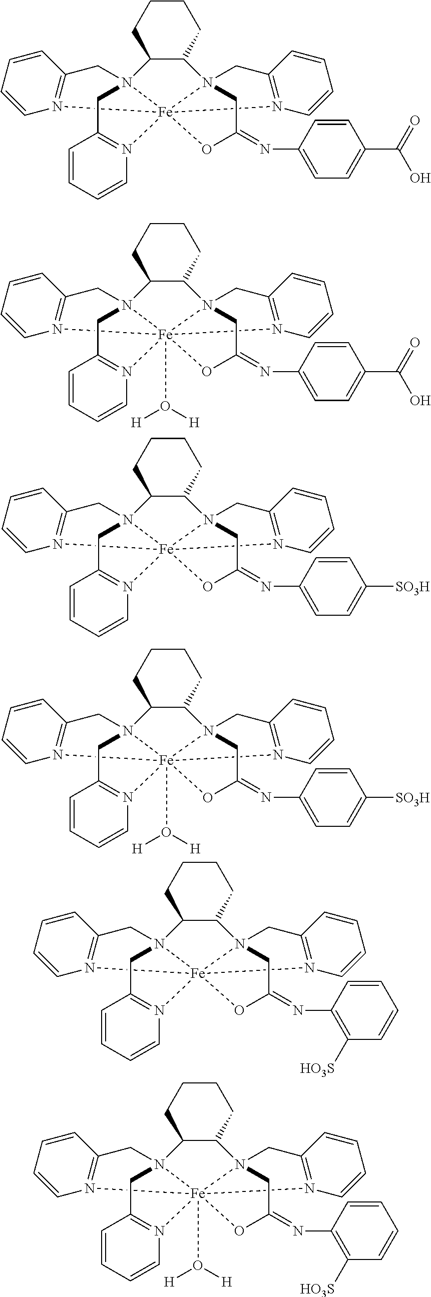

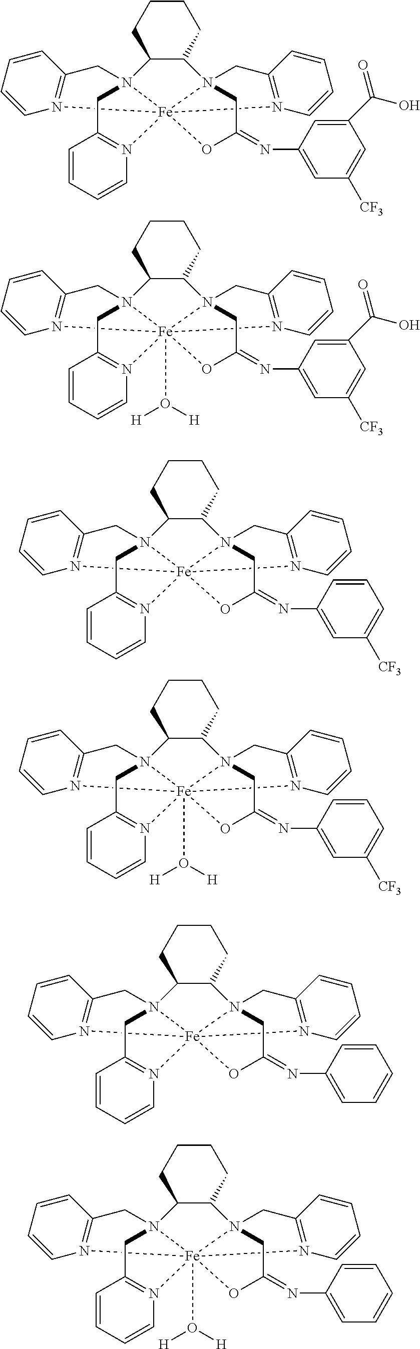

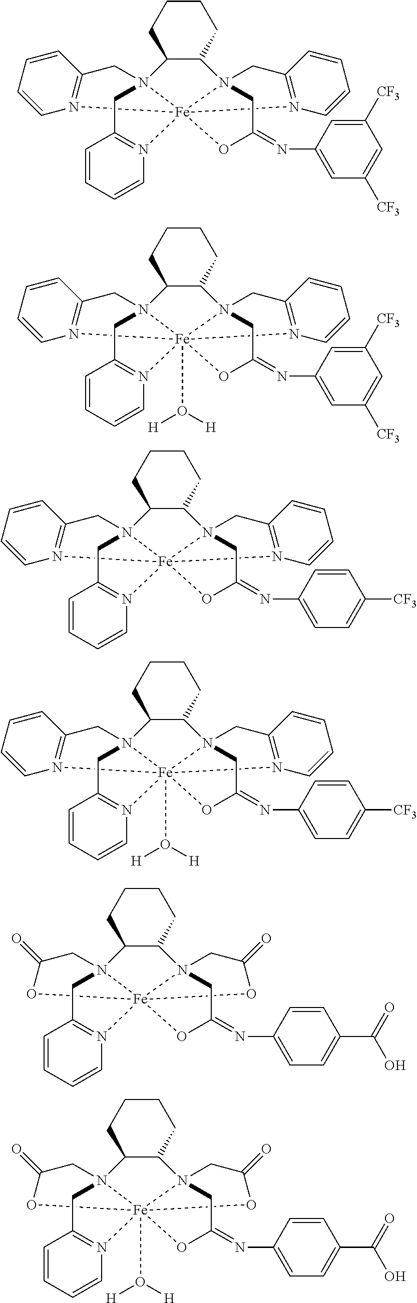

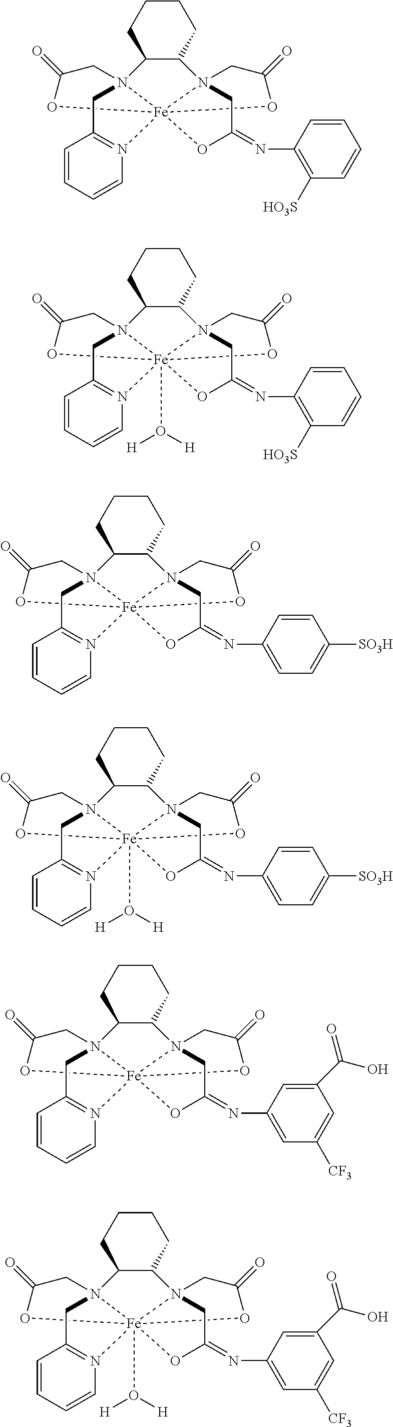

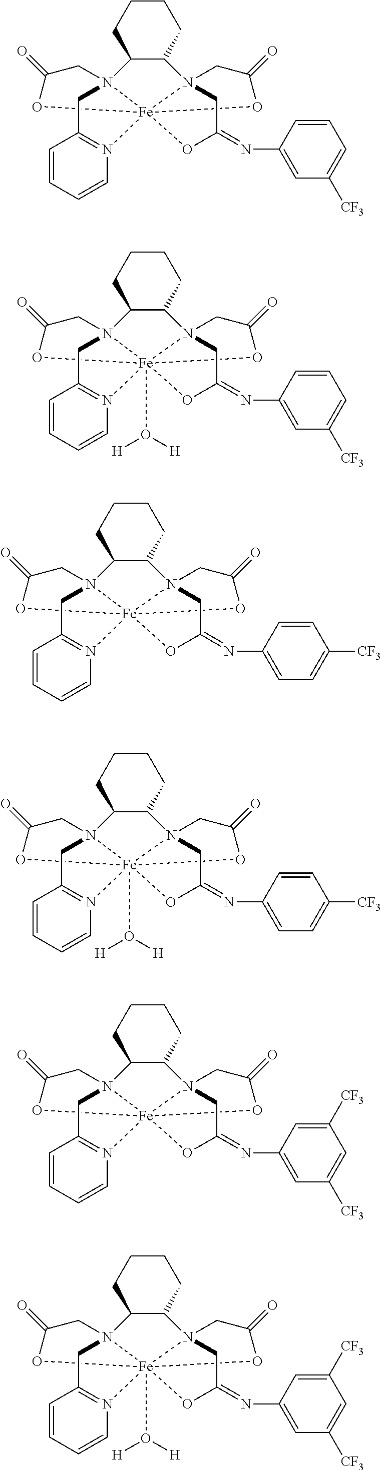

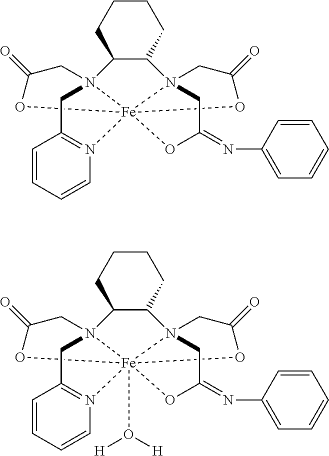

or a pharmaceutically acceptable salt thereof.
109. A pharmaceutical composition comprising a compound of any one of embodiments 59-108, or a pharmaceutically acceptable salt thereof, and a pharmaceutically acceptable carrier.
110. A method of magnetic resonance (MR) imaging of an organ or a tissue of a subject, the method comprising:
   i) administering to the subject an effective amount of a compound of any one of embodiments 59-108, or a pharmaceutically acceptable salt thereof, or a pharmaceutical composition of embodiment 109;
   ii) waiting a time sufficient to allow the compound to accumulate in the organ or a tissue to be imaged; and
   ii) acquiring an MR image of the organ or the tissue of the subject.
111. The method of embodiment 110, wherein the organ or the tissue is selected from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a cartilage, an epithelium, a tendon, and a ligament.
112. A method of diagnosing a disease or condition in a subject, the method comprising:
   i) administering to the subject an effective amount of a compound of any one of embodiments 59-108, or a pharmaceutically acceptable salt thereof, or a pharmaceutical composition of embodiment 109;
   ii) waiting a time sufficient to allow the compound to accumulate in an organ or a tissue of the subject affected by the disease or condition; and
   iii) acquiring an MR image of the organ or the tissue of the subject, wherein observing the MR image attributable to the compound of Formula (B) is indicative of the disease or condition.
113. A method of monitoring treatment of a disease or condition in a subject, the method comprising:
   i) administering to the subject an effective amount of a compound of any one of embodiments 59-108, or a pharmaceutically acceptable salt thereof, or a pharmaceutical composition of embodiment 109;
   ii) waiting a time sufficient to allow the compound to accumulate in an organ or tissue of the subject affected by the inflammatory disease or condition;
   iii) acquiring a first MR image of the organ or the tissue of the subject;
   iv) administering to the subject a therapeutic agent in an effective amount to treat the disease or condition;
   v) after iv), administering to the subject an effective amount of a compound of any one of embodiments 59-108, or a pharmaceutically acceptable salt thereof, or a pharmaceutical composition of embodiment 109;
   vi) waiting a time sufficient to allow the compound to accumulate in the organ or tissue of the subject affected by the disease or condition;
   vii) acquiring a second MR image of the organ or the tissue of the subject; and
   viii) comparing the first and the second MR images,
   wherein observing a difference between the first and the second MR images attributable to the compound of Formula (B) is indicative of progression of treatment of the disease or condition.
114. The method of embodiment 112 or embodiment 113, wherein the disease or condition is selected from cancer, blood clot, brain lesion, disrupted blood-brain-barrier, arterial stenosis, and spinal stenosis.
115. A compound of Formula (I):

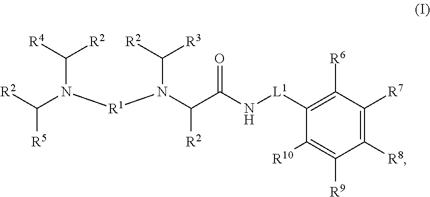

or a pharmaceutically acceptable salt thereof, wherein:
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₆ alkyl)_{q}SO₂NR^{A}R^{B}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)OR^{A}, P(O)(OR^{A})(B), (C═O)NR^{A}R^{B}; and ring B, which is optionally substituted with 1, 2, or 3 substituents independently selected from R^{1B};
R⁴ is selected from C(O)OR^{A}, P(O)(OR^{A})(OR^{B}), (C═O)NR^{A}R^{B}; and ring C, which is optionally substituted with 1, 2, or 3 substituents independently selected from R^{1C};
R⁵ is selected from CO(OR^{A}), P(O)(OR^{A})(OR^{B}), (C═O)NR^{A}R^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl;
with a proviso that the compound of Formula (I) is not:

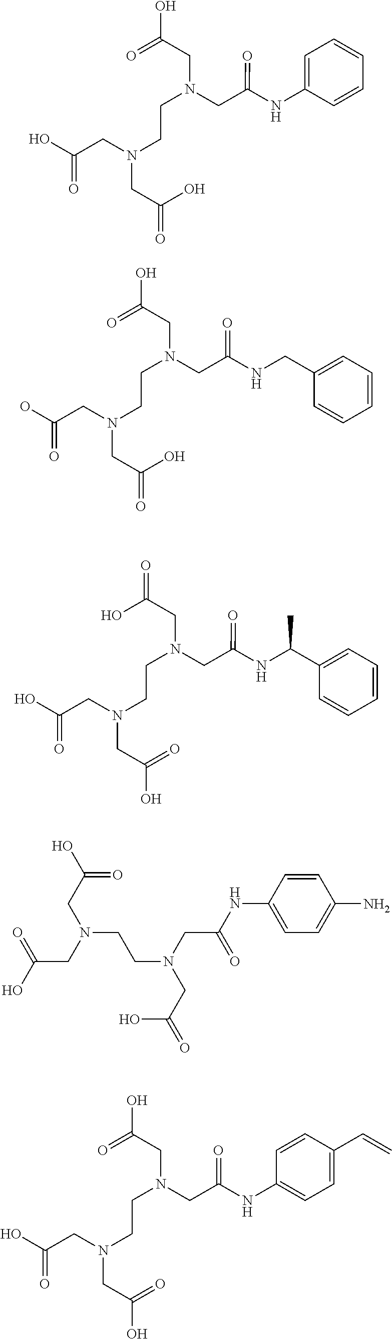

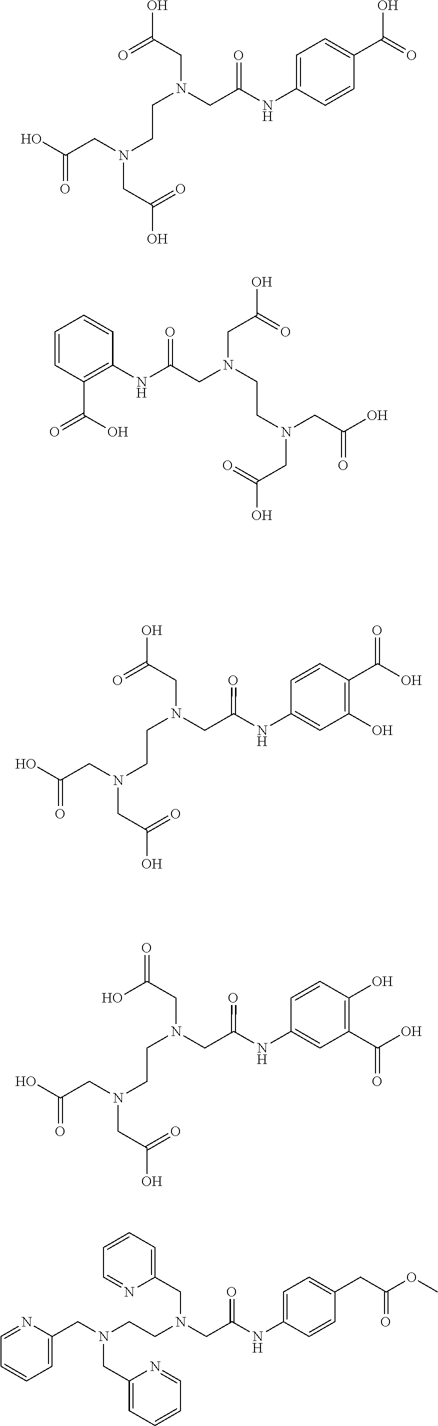 [SMILES: O=C(O)CN(CCN(CC(=O)O)CC(=O)Nc1ccc(C(=O)O)cc1)CC(=O)O ; O=C(O)CN(CCN(CC(=O)O)CC(=O)Nc1ccccc1C(=O)O)CC(=O)O ; O=C(O)CN(CCN(CC(=O)O)CC(=O)Nc1ccc(C(=O)O)c(O)c1)CC(=O)O ; O=C(O)CN(CCN(CC(=O)O)CC(=O)Nc1ccc(O)c(C(=O)O)c1)CC(=O)O ; COC(=O)Cc1ccc(NC(=O)CN(CCN(Cc2ccccn2)Cc2ccccn2)Cc2ccccn2)cc1]

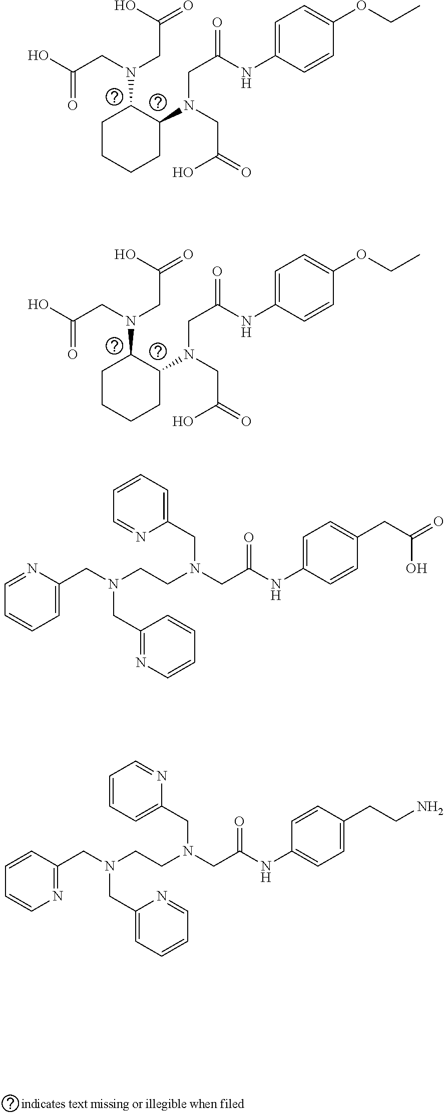 [SMILES: CCOc1ccc(NC(=O)CN(CC(=O)O)[C@H]2CCCC[C@@H]2N(CC(=O)O)CC(=O)O)cc1 ; CCOc1ccc(NC(=O)CN(CC(=O)O)[C@@H]2CCCC[C@H]2N(CC(=O)O)CC(=O)O)cc1 ; O=C(O)Cc1ccc(NC(=O)CN(CCN(Cc2ccccn2)Cc2ccccn2)Cc2ccccn2)cc1 ; NCCc1ccc(NC(=O)CN(CCN(Cc2ccccn2)Cc2ccccn2)Cc2ccccn2)cc1]

or a pharmaceutically acceptable salt thereof.
116. The compound of embodiment 115, wherein L¹ is C₁₋₃ alkylene.
117. The compound of embodiment 116, wherein L¹ is methylene.
118. The compound of embodiment 115, wherein L¹ is absent.
119. The compound of any one of embodiments 115-118, wherein R¹ is C₁₋₃ alkylene-ring A-C₁₋₃ alkylene, wherein said ring A is optionally substituted with 1, 2, or 3 substituents independently selected from R^{1A}.
120. The compound of any one of embodiments 115-118, wherein the compound has formula.

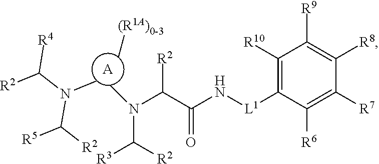

or a pharmaceutically acceptable salt thereof.
121. The compound of embodiment 120, wherein ring A is phenylene, optionally substituted with R^{1A}.
122. The compound of embodiment 120, wherein ring A is 5-6 membered heteroarylene, optionally substituted with R^{1A}.
123. The compound of embodiment 120, wherein ring A is C₃-C₁₀ cycloalkylene, optionally substituted with R^{1A}.
124. The compound of embodiment 123, wherein ring A is cyclopentylene or cyclohexylene, optionally substituted with R^{1A}.
125. The compound of embodiment 124, wherein ring A is a moiety of formula:

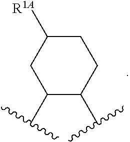

126. The compound of embodiment 120, wherein ring A is 4-10 membered heterocycloalkylene, optionally substituted with R^{1A}.
127. The compound of any one of embodiments 115-118, wherein R¹ is C₂₋₆ alkylene, optionally substituted with R^{1A}.
128. The compound of embodiment 127, wherein R¹ is 1,2-ethylene, optionally substituted with R^{1A}.
129. The compound of embodiment 128, wherein R¹ is a moiety of formula:

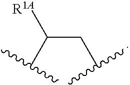

130. The compound of any one of embodiments 115-129, wherein each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
131. The compound of embodiment 124, wherein ring A is selected from any one of the following moieties:

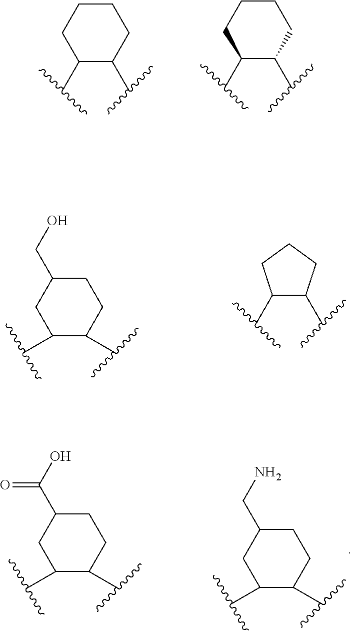 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

132. The compound of embodiment 126, wherein ring A is selected from any one of the following moieties:

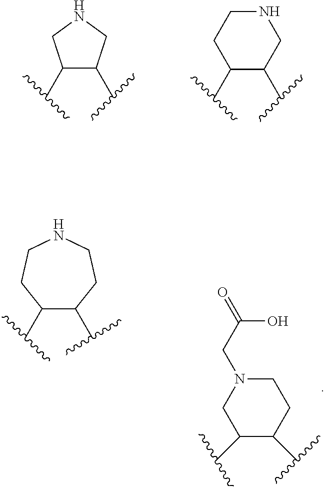 [SMILES: *C1CNCC1* ; *C1CCNCC1* ; *C1CCNCCC1* ; *C1CCN(CC(=O)O)CC1*]

133. The compound of embodiment 128, wherein R¹ is selected from any one of the following moieties:

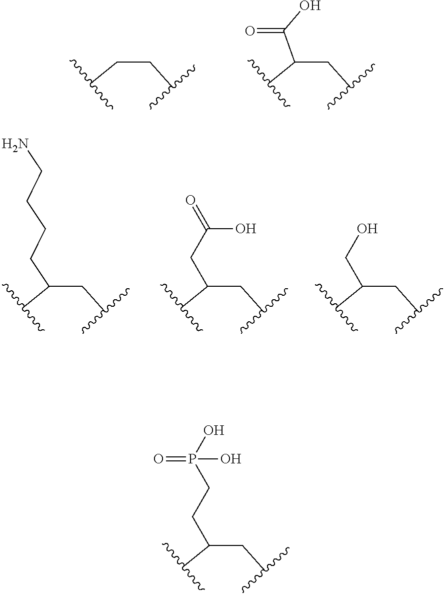

134. The compound of any one of embodiments 115-133, wherein each R² is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
135. The compound of embodiment 134, wherein each R² is H.
136. The compound of embodiment 134, wherein at least one R² is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).
137. The compound of any one of embodiments 115-118, wherein the compound has formula:

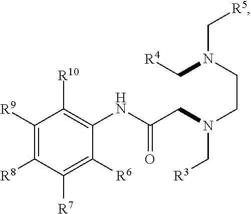

or a pharmaceutically acceptable salt thereof.
138. The compound of any one of embodiments 115-118, wherein the compound has formula:

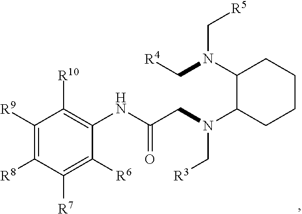

or a pharmaceutically acceptable salt thereof.
139. The compound of any one of embodiments 115-138, wherein R³ is ring B, optionally substituted with 1, 2, or 3 substituents independently selected from R^{1B}.
140. The compound of any one of embodiments 115-139, wherein R⁴ is ring C, optionally substituted with 1, 2, or 3 substituents independently selected from R^{1C}.
141. The compound of any one of embodiments 115-140, wherein R⁵ is ring D, optionally substituted with 1, 2, or 3 substituents independently selected from R^{1D}.
142. The compound of embodiment 139, wherein ring B is 5-6 membered heteroaryl optionally substituted with 1, 2, or 3 substituents independently selected from R^{1B}.
143. The compound of embodiment 140, wherein ring C is 5-6 membered heteroaryl optionally substituted with 1, 2, or 3 substituents independently selected from R^{1C}.
144. The compound of embodiment 141, wherein ring D is 5-6 membered heteroaryl optionally substituted with 1, 2, or 3 substituents independently selected from R^{1D}.
145. The compound of any one of embodiments 142-144, wherein the 5-6 membered heteroaryl is selected from any one of the following moieties:

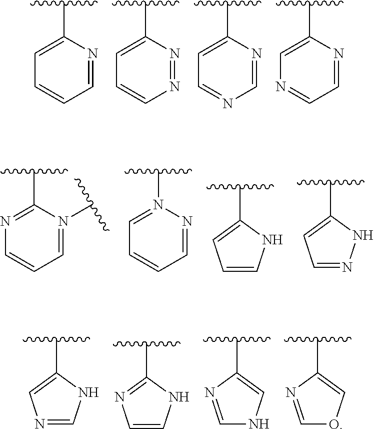

146. The compound of embodiment 139, wherein ring B is 5-6 membered heterocycloalkyl, optionally substituted with 1, 2, or 3 substituents independently selected from R^{1B}.
147. The compound of embodiment 140, wherein ring C is 5-6 membered heterocycloalkyl optionally substituted with 1, 2, or 3 substituents independently selected from R^{1C}.
148. The compound of embodiment 141, wherein ring D is 5-6 membered heterocycloalkyl optionally substituted with 1, 2, or 3 substituents independently selected from R^{1D}.
149. The compound of any one of embodiments 146-148, wherein the 5-6 membered heterocycloalkyl is selected from any one of the following moieties:

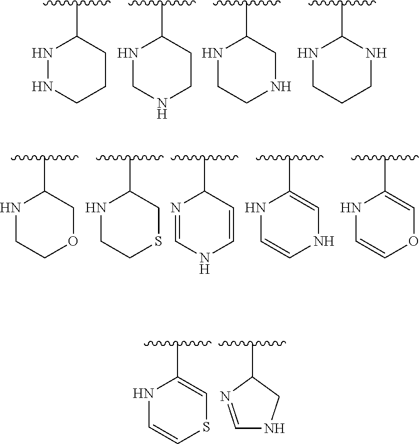

150. The compound of any one of embodiments 115-149, wherein each R^{1B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
151. The compound of any one of embodiments 115-150, wherein each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
152. The compound of any one of embodiments 115-151, wherein each RD is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
153. The compound of any one of embodiments 115-138, wherein R³ is C(O)OR^{A}.
154. The compound of any one of embodiments 115-138, wherein R⁴ is C(O)OR^{A}.
155. The compound of any one of embodiments 115-138, wherein R⁵ is C(O)OR^{A}.
156. The compound of embodiment 137, having formula:

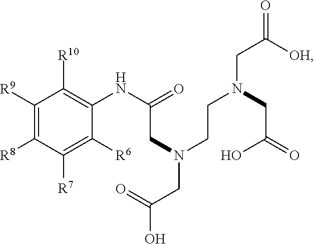

or a pharmaceutically acceptable salt thereof.
157. The compound of embodiment 138, having formula:

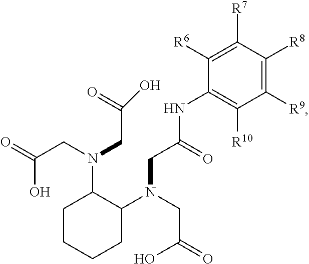

or a pharmaceutically acceptable salt thereof.
158. The compound of any one of embodiments 115-157, wherein R⁶, R⁷, R⁸, R⁹, and R¹⁰ are independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
159. The compound of any one of embodiments 115-157, wherein at least one of R⁶, R⁷, R⁸, R⁹, and R¹⁰ is selected from OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
160. The compound of any one of embodiments 115-157, wherein at least one of R⁶, R⁷, R⁸, R⁹, and R¹⁰ is selected from halo, C₁₋₆ haloalkyl, C₁₋₆ haloalkoxy.
161. The compound of any one of embodiments 115-157, wherein at least one of R⁶, R⁷, R⁸, R⁹, and R¹⁰ is selected from —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).
162. The compound of any one of embodiments 115-157, wherein:
   R⁶, R⁸, and R¹⁰ are each H, and
   R⁷ and R⁹ are each independently selected from halo, C₁₋₆ haloalkyl, and C₁₋₆ haloalkoxy.
163. The compound of embodiment 115, having formula:

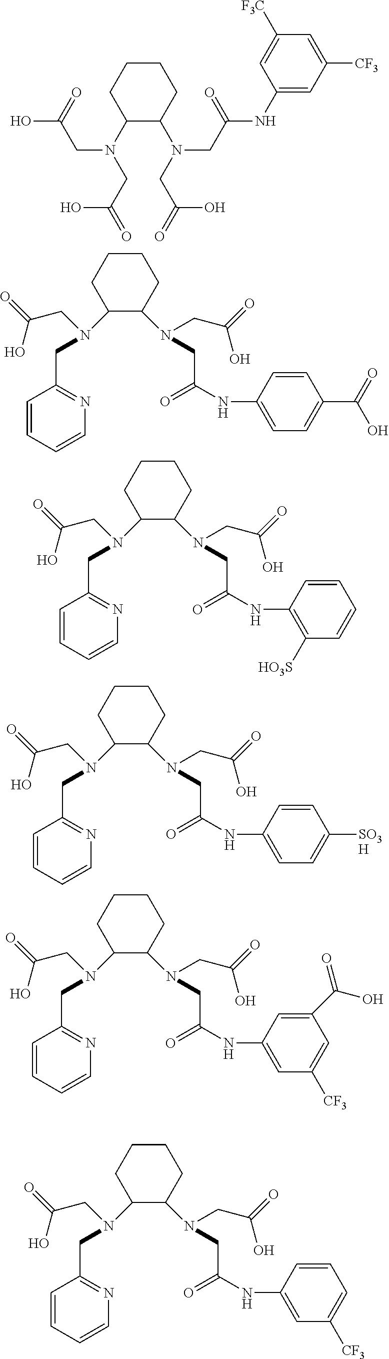

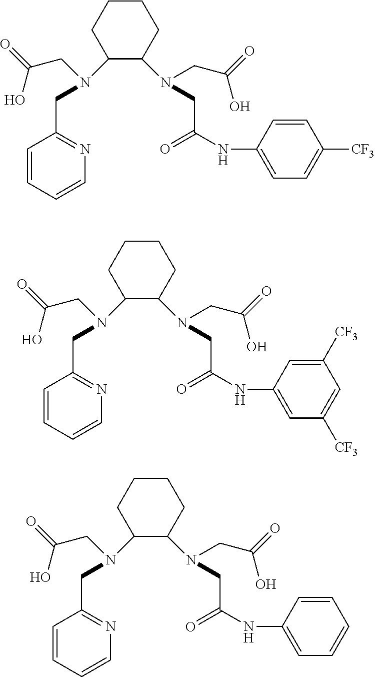

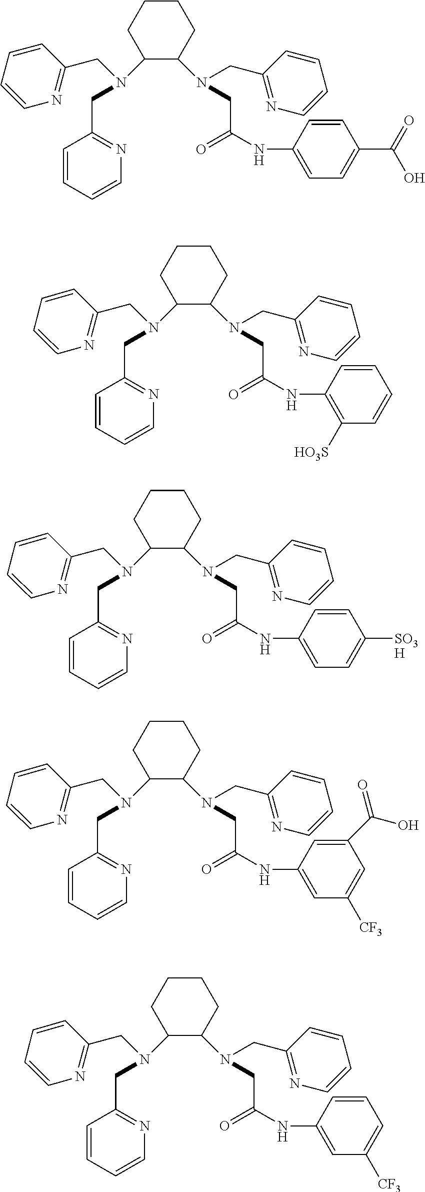

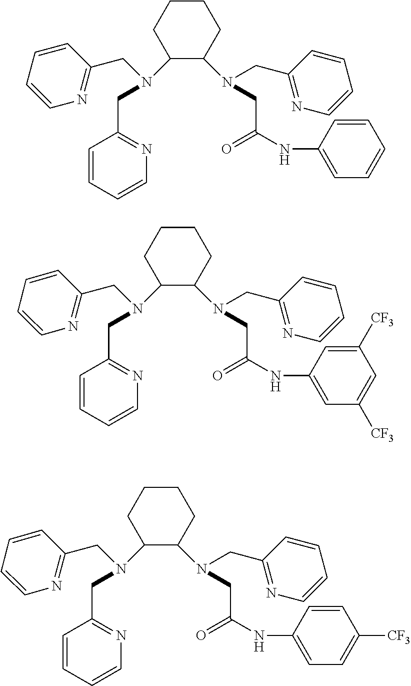

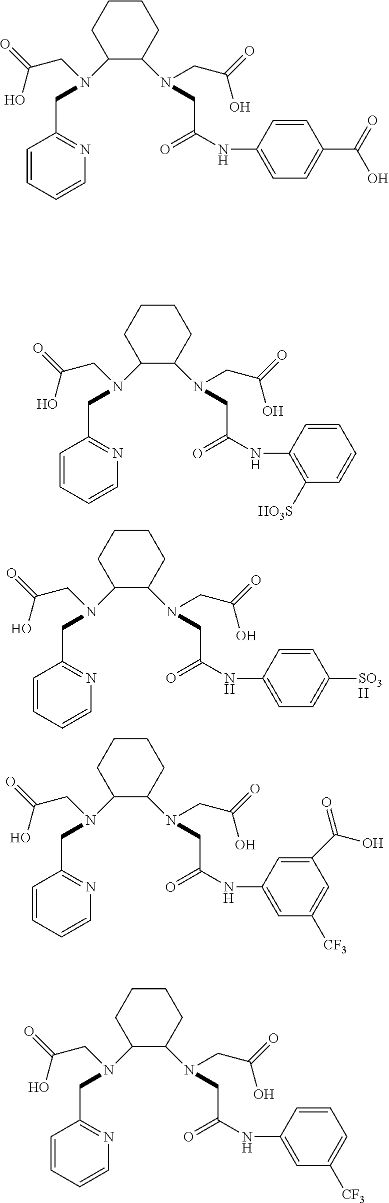

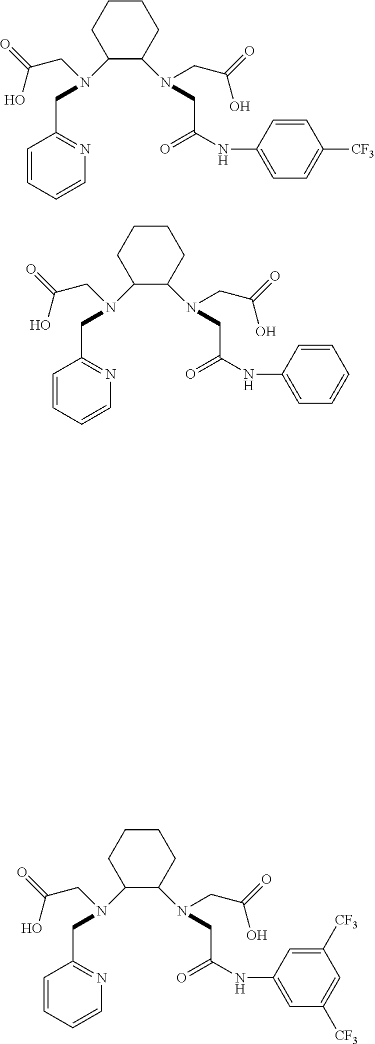

or a pharmaceutically acceptable salt thereof.

### EXAMPLES

The following examples are for illustrative purposes only and are not limiting.

### Example 1—Preparation of Fe(III) Complex of CDTA-pH-3,5-CF₃

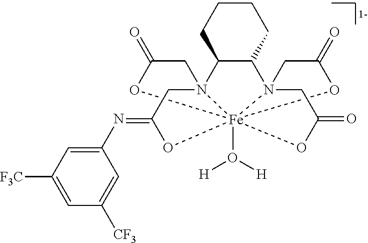

### Step 1—synthesis of 2,2′-((2-((2-((3,5-bis(trifluoromethyl)phenyl)amino)-2-oxoethyl)(carboxymethyl)amino)cyclohexyl)azanediyl)diacetic acid (CDTA-Ph-3,5-CF₃)

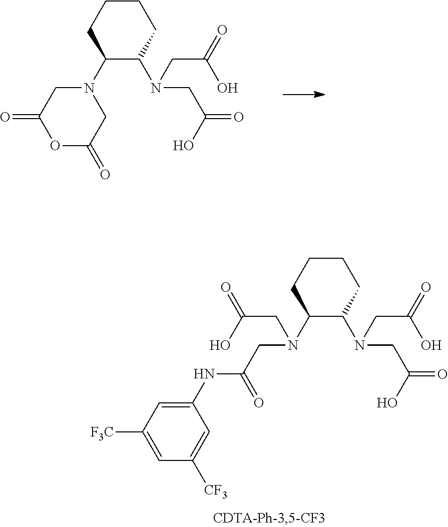

1.312 g (4 mmol) of CDTA-Anhydride was added to 5 mL DMF followed by 702 μL (4.4 mmol) of 3,5-CF₃-Aniline and 613 μL (4.4 mmol) of triethylamine. The reaction slurry was left to stir overnight. The reaction mixture was then transferred into a separate vial where 50 mL of ether was added to crash out the product, followed by centrifugation. The resulting solid was then washed and centrifuged with ether 2 more times. Product was purified using a 100 g C18 RediSep column with a 5-100% ACN (0.1% TFA) gradient over 15 column volumes at 60 mL/min. Lyophilizing the combined fractions yielded the ligand as a white powder with 0.86 mol equiv. TFA associated (96 8 mg, 1.50 mmol, 38%). ¹H NMR (500 MHz, D₂O) δ: 7.905 (d, 2H, J=5 Hz), 7.75 (s, 1H), 3.04 (m, 8H), 2.4 (m, 2H), 2.0 (m, 2H), 1.69 (s, 2H), 1.08 (m, 4H). ¹³C NMR (500 MHz, D₂O) δ: 180.3, 174.7, 168.5, 162.87 (q), 150.39, 130.9 (q), 126.9, 124.8, 123.7, 122.6, 120.4, 119.8, 117.5, 115.2, 112.8, 60.7, 25.2, 23.54. ¹⁹F NMR (500 MHz, D₂O) δ: 62.3 (s, 6F), 75.4 (s, 8.05F). ESI-MS for [C22H25F6N3O7+H]⁺: m/z [M+H]=558.16 (calc.); m/z=558.2 (obsv.)

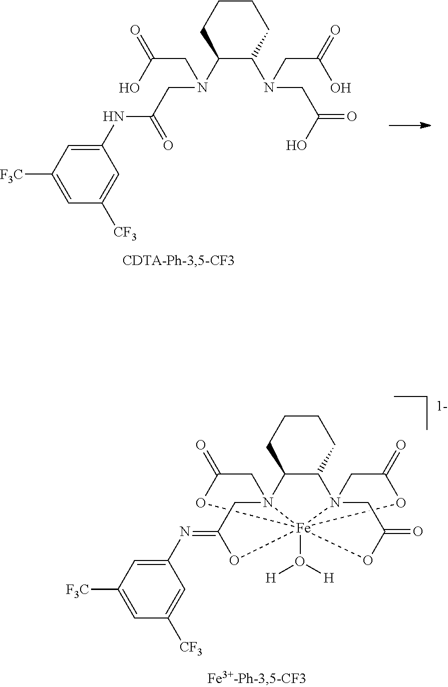

### Step 2—Synthesis of Fe³⁺ Complex of CDTA-Ph-3,5-CF₃

The title compound was synthesized in situ by mixing the compound obtained in step 1 with 0.5 molar equiv. Fe₂(SO₄)₃, (i.e., 1 molar equiv. Fe³⁺). In one representative sample preparation, used for pH-potentiometry measurements, 7.2 mg (0.011 mmol) Ligand were mixed with 4.4 mg (0.011 mmol Fe) were mixed in 3.4 mL water (with 0.1 M NaCl), and the pH adjusted using 0.1 M NaOH. ESI-MS for [C22H22F6FeN3O7]⁻: m/z [M⁻]=609.1 (calc.); m/z=609.1(obsv.)

### Example 2—pH Potentiometric Titration of CDTA-pH-3,5-CF₃ the Corresponding Fe(III) Complex

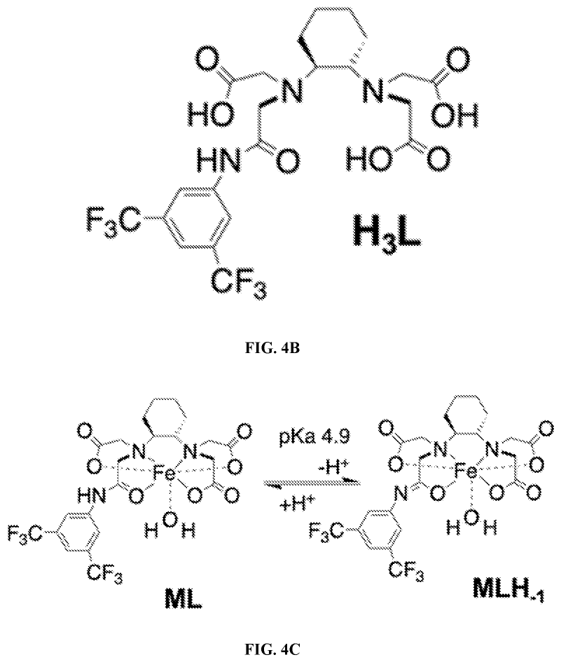

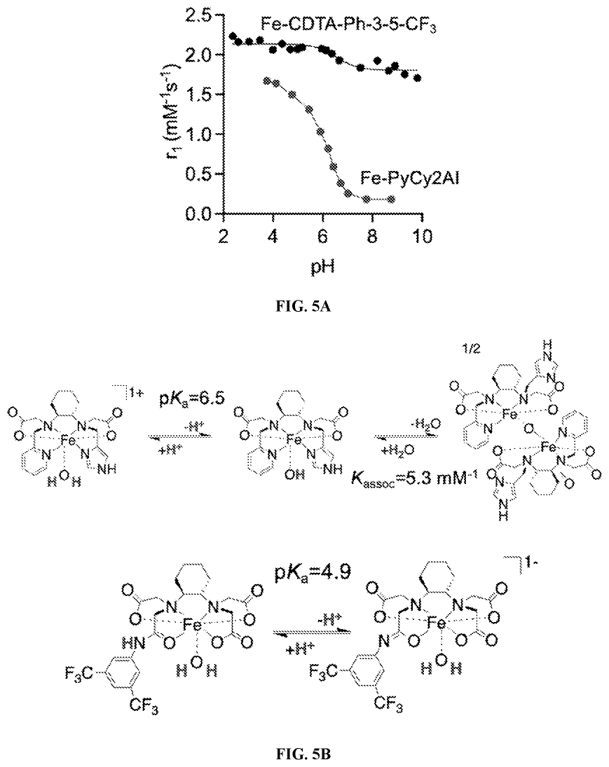

FIG. 4, Panel A, shows pH-potentiometric titration data of the ligand CDTA-Ph-3,5-CF₃ in the absence (denoted by L) and presence of 1 molar equiv Fe³⁺ (denoted by ML). Panel B shows putative structures of the ligand in H3L form. Panel B shows the complex in ML and MLH₋₁ forms. From the titration data (performed in combination with EDTA competition assay at pH 3), a log K=23.7 s estimated for Fe-CDTA-Ph-3,5-CF₃. The titration data show that after formation of ML, another 1 molar equivalent proton is consumed with estimated pKa 4.9, resulting in MLH₋₁. The proposed MLH₋₁ structure assignment is supported by the data shown in FIGS. 5-7.

### Example 3—Fe³⁺-CDTA-pH-3,5-CF₃ Relaxivity as a Function of pH

FIG. 5, Panel A, compares the pH-dependence on T₁-relaxivity (r₁; the Fe induced increase in T1 relaxation rate (R1) normalized to Fe concentration in mM) for the structurally related Fe³⁺ complexes Fe-CDTA-Ph-3,5-CF₃ and Fe-PyCy2AI. Panel B shows chemical equilibria that govern speciation of either complex within the range of measured pH values. Note the large drop in r₁ that accompanies the pKa 6.5 deprotonation event of Fe-PyC3A, resulting from formation of the low-r₁ μ-oxo-bridge and antiferromagnetically coupled dimeric complex. Note how Fe-CDTA-Ph-3,5-CF₃ r₁ at pH 7.4, where speciation is dominated by the monomeric complex, is much greater and consistent with values previously reported for high-spin complexes of Fe³⁺. The imidate donor increases the pKa of the Fe³⁺ water co-ligand to values>7.4, which stabilizes the monomeric Fe³⁺ complex against formation of μ-oxo-bridged and antiferromagnetically coupled complexes at physiologically relevant pH.

### Example 4—Variable Temperature ¹⁷O T2-Relaxometry Recorded in Aqueous Solution in the Absence and Presence of Fe³⁺-CDTA-pH-3,5-CF₃

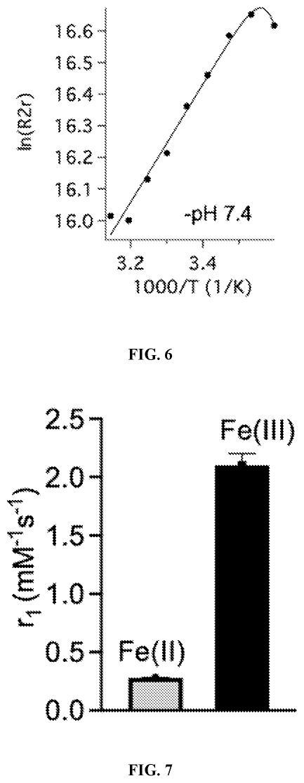

Fe-CDTA-Ph-3,5-CF₃ substantially increases the T2-relaxation rate (R₂) of water-¹⁷O nuclei, consistent with the presence of a rapidly exchanging water co-ligand. FIG. 6 plots the reduced relaxation rate (R₂ᵣ; defined as the Fe induced R₂ increase vs neat water, normalized to the mole fraction of water molecules coordinated to Fe) as a function of temperature.

### Example 5—Fe³⁺-CDTA-Ph-3,5-CF₃ is 10-Fold Greater than Fe²⁺-CDTA-Ph-3,5-CF₃

FIG. 7 compares the relaxivity (r₁; the Fe induced increase in T1 relaxation rate (R1) normalized to Fe concentration in mM) recorded for Fe³⁺-CDTA-Ph-3,5-CF₃ and Fe²⁺-CDTA-Ph-3,5-CF₃ at pH 7.4. The Fe²⁺ complex was formed in situ by addition of excess sodium ascorbate. The r₁ values for the Fe³⁺ complex (2.1±0.11 mM⁻¹s⁻¹) is 7.5-times greater than the sister Fe²⁺ complex (2.8±0.003 mM⁻¹s⁻¹).

### Example 6—Cyclic Voltammetry of Fe-CDTA-Ph-3,5-CF₃

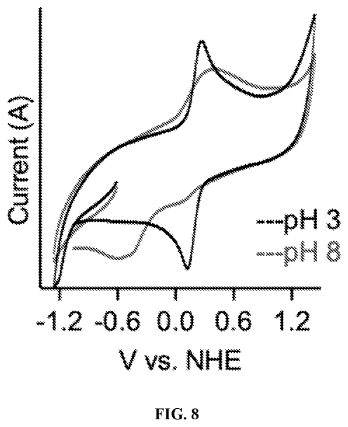

FIG. 8 shows cyclic voltammograms (CV) of Fe-CDTA-Ph-3,5-CF₃ recorded at pH 3 (where the Fe³⁺ complex is in predominantly ML speciation, and pH 9, where the Fe³⁺ complex is in the predominantly MLH₋₁ form (0.5 M KNO3 electrolyte, GC working electrode, Ag/AgCl reference electrode, Pt counter electrode). The CV data recorded at pH3 shows reversible oxidation and reduction waves, consistent with little structural rearrangement upon switching between the Fe³⁺ and Fe²⁺ oxidation states. However, at pH 8 the oxidation and reduction waves are separated by nearly 1V, consistent with a structural change upon oxidation of the Fe²⁺ complex to a highly Fe³⁺ stabilizing structure.

### OTHER EMBODIMENTS

It is to be understood that while the present application has been described in conjunction with the detailed description thereof, the foregoing description is intended to illustrate and not limit the scope of the present application, which is defined by the scope of the appended embodiments. Other aspects, advantages, and modifications are within the scope of the following embodiments.

1. A compound of Formula (A): 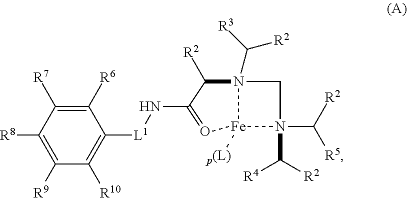
or a pharmaceutically acceptable salt thereof, wherein: [symbol]
indicates a coordinate bond;
each L is independently a Fe ligand;
p is 0, 1 or 2;
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}SO₂NR^{A}R^{B}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl;
with a proviso that the compound of Formula (A) is not:

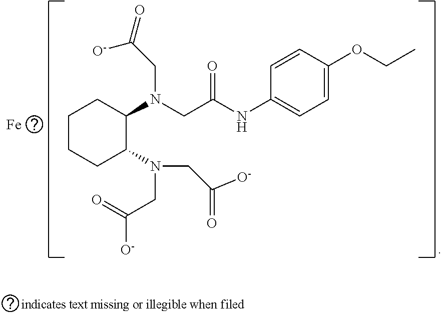

2. The compound of claim 1, wherein L¹ is C₁₋₃ alkylene.

3. The compound of claim 2, wherein L¹ is methylene.

4. The compound of claim 1, wherein L¹ is absent.

5. The compound of claim 1, wherein R¹ is C₁₋₃ alkylene-ring A-C₁₋₃ alkylene, wherein said ring A is optionally substituted with 1, 2, or 3 substituents independently selected from R^{1A}.

6. The compound of claim 1, wherein the compound has formula: 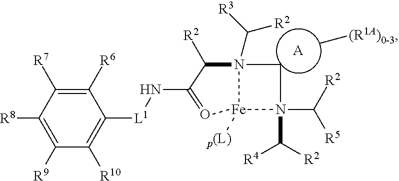
or a pharmaceutically acceptable salt thereof.

7. The compound of claim 1, wherein the compound has formula: 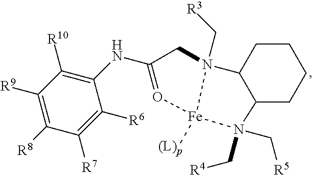
or a pharmaceutically acceptable salt thereof.

8. The compound of claim 1, wherein the compound has formula: 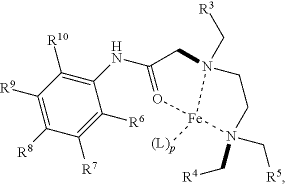
or a pharmaceutically acceptable salt thereof.

9. The compound of claim 8, having formula: 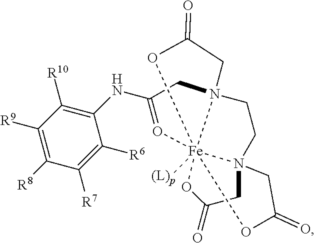
or a pharmaceutically acceptable salt thereof.

10. The compound of claim 1, having formula: 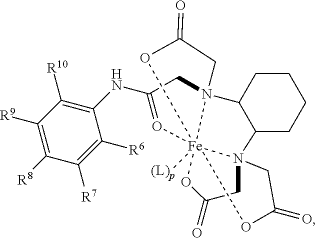
or a pharmaceutically acceptable salt thereof.

11. The compound of claim 1, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

12. The compound of claim 1, selected from any one of the following compounds: 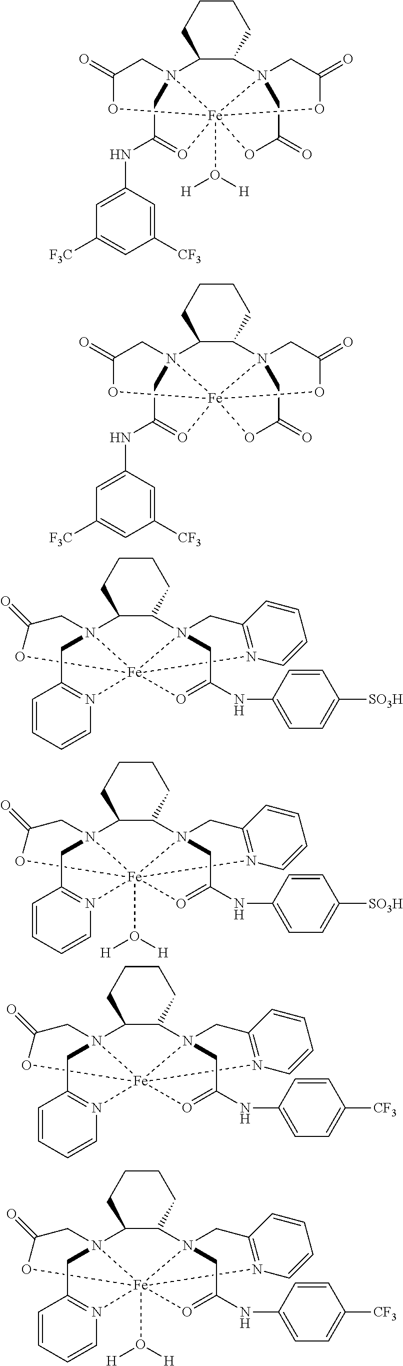 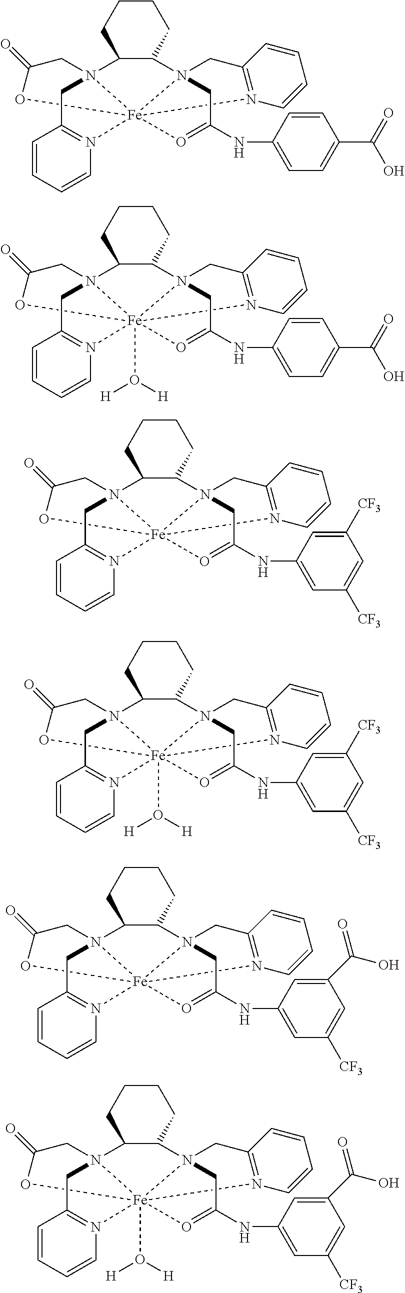 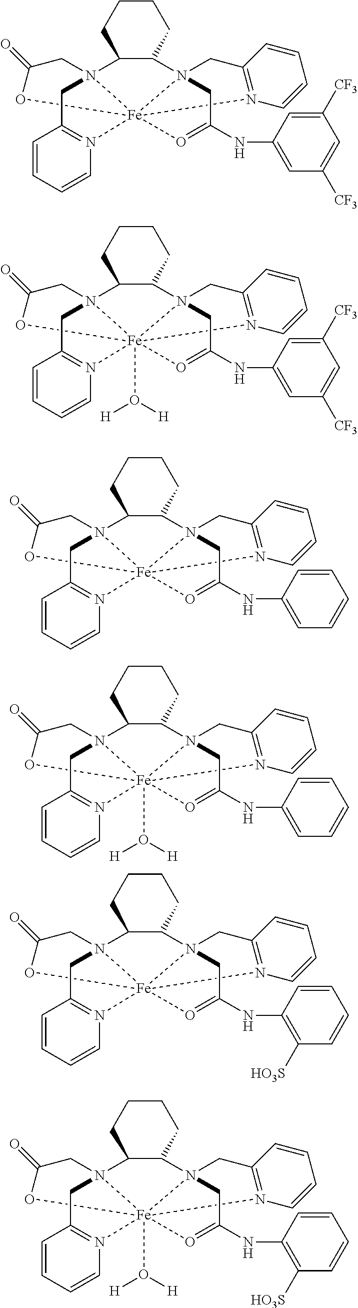 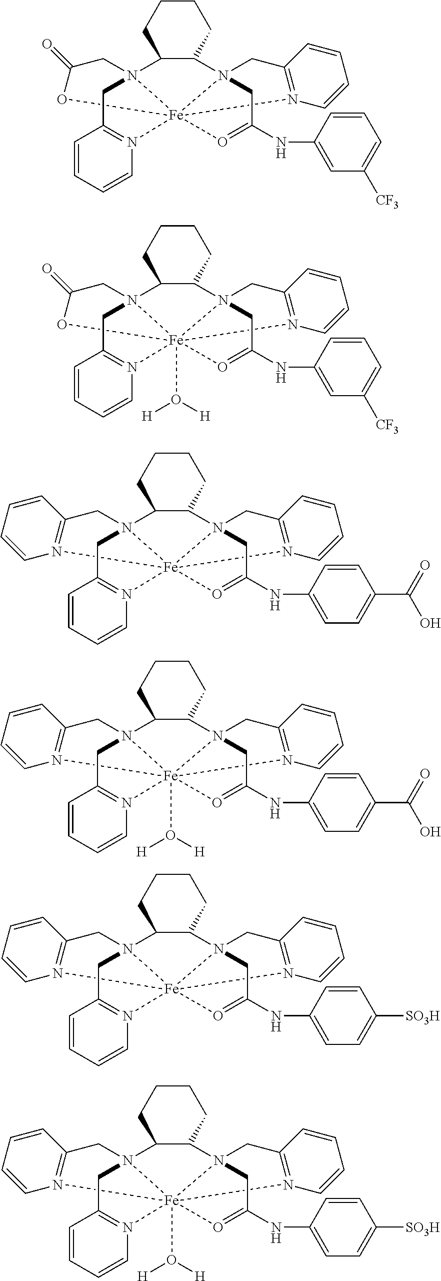 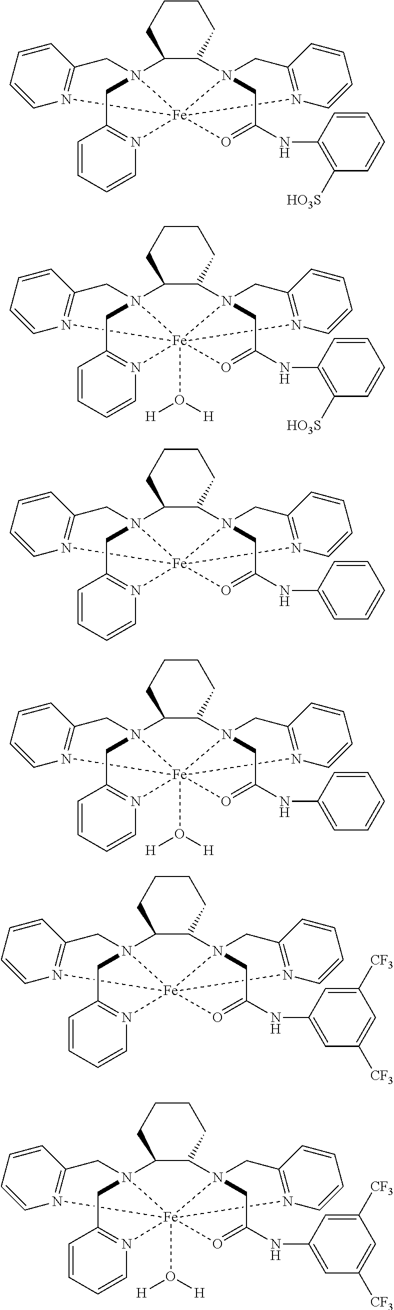 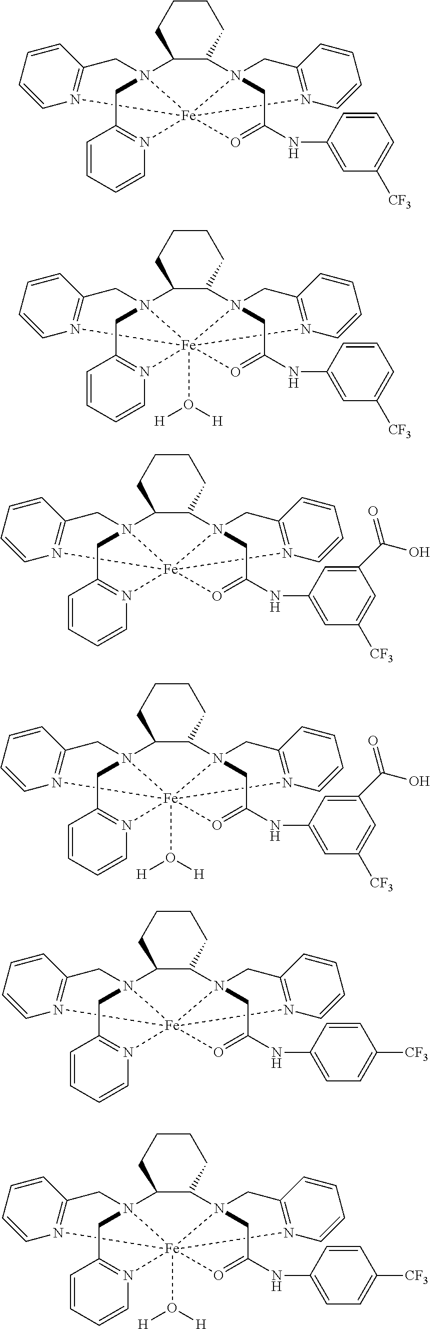 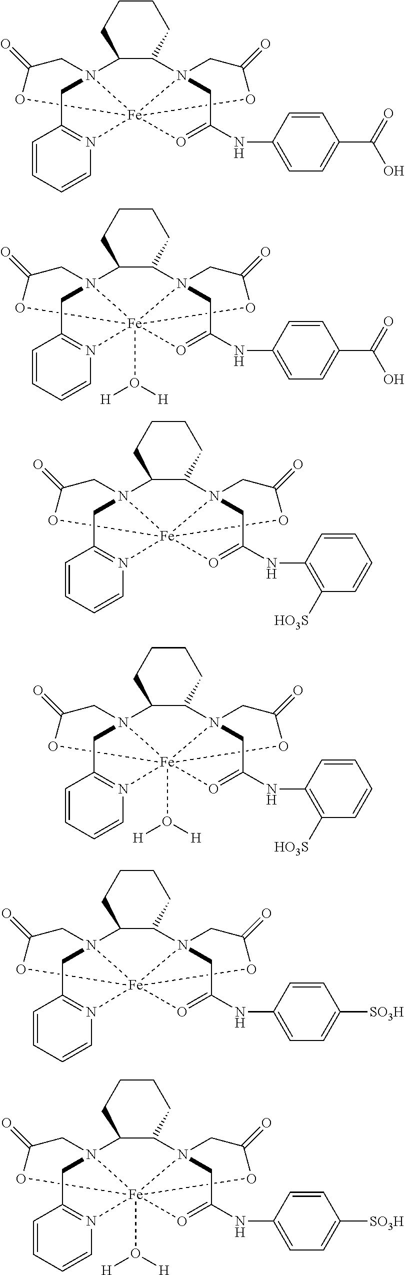 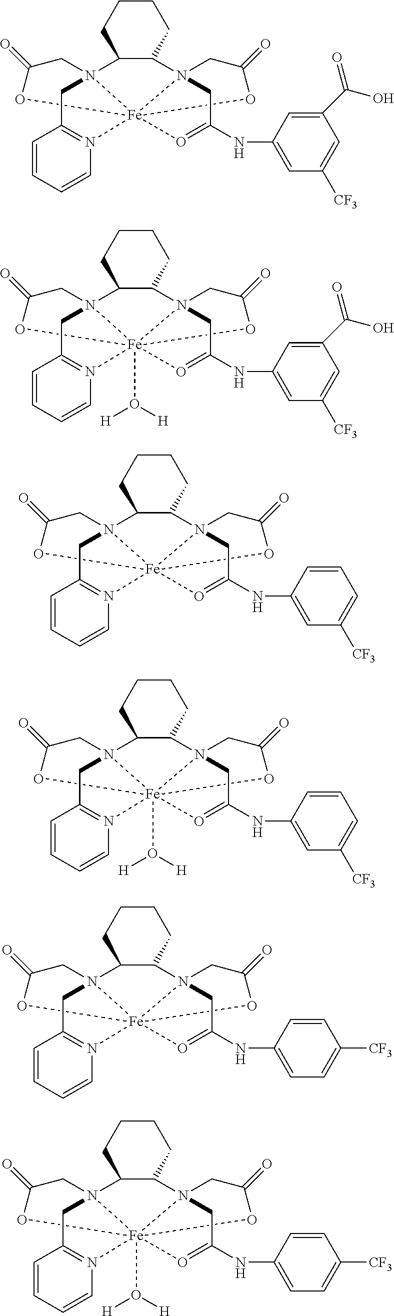 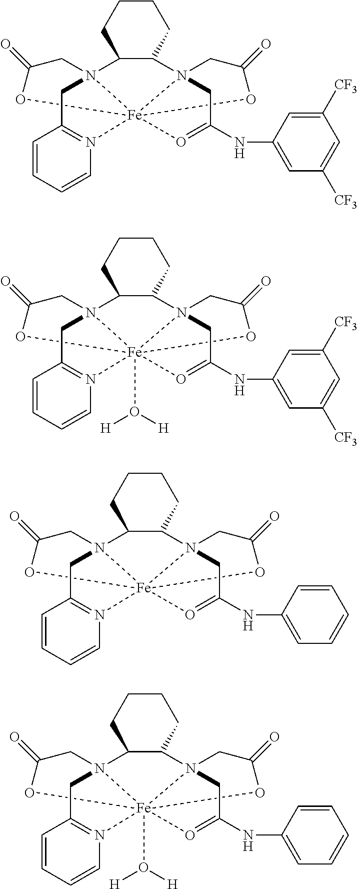
or a pharmaceutically acceptable salt thereof.

13. A pharmaceutical composition comprising a compound of claim 1, or a pharmaceutically acceptable salt thereof, and a pharmaceutically acceptable carrier.

14. A method of magnetic resonance (MR) imaging of an organ or a tissue of a subject, the method comprising:
i) administering to the subject an effective amount of a compound of claim 1, or a pharmaceutically acceptable salt thereof;
ii) waiting a time sufficient to allow the compound to accumulate in the organ or a tissue to be imaged; and
ii) acquiring an MR image of the organ or the tissue of the subject.

15. The method of claim 14, wherein the organ or the tissue comprises an area affected by acute or chronic inflammation.

16. A method of diagnosing an inflammatory disease or condition in a subject, the method comprising:
i) administering to the subject an effective amount of a compound of claim 1, or a pharmaceutically acceptable salt thereof;
ii) waiting a time sufficient to allow the compound to accumulate in an organ or a tissue of the subject affected by the inflammatory disease or condition; and
iii) acquiring an MR image of the organ or the tissue of the subject,
wherein observing the MR image attributable to the compound of Formula (A) is indicative of the inflammatory disease or condition.

17. A method of monitoring treatment of an inflammatory disease or condition in a subject, the method comprising:
i) administering to the subject an effective amount of a compound of claim 1, or a pharmaceutically acceptable salt thereof;
ii) waiting a time sufficient to allow the compound to accumulate in an organ or tissue of the subject affected by the inflammatory disease or condition;
iii) acquiring a first MR image of the organ or the tissue of the subject;
iv) administering to the subject a therapeutic agent in an effective amount to treat the inflammatory disease or condition;
v) after iv), administering to the subject an effective amount of a compound of claim 1, or a pharmaceutically acceptable salt thereof;
vi) waiting a time sufficient to allow the compound to accumulate in the organ or tissue of the subject affected by the inflammatory disease or condition;
vii) acquiring a second MR image of the organ or the tissue of the subject; and
viii) comparing the first and the second MR images,
wherein observing a difference between the first and the second MR images attributable to the compound of Formula (A) is indicative of progression of treatment of inflammatory disease or condition.

18. The method of claim 16, wherein the inflammatory disease or condition is selected from cancer, kidney failure, diabetes, rheumatoid arthritis, osteoarthritis, inflammatory joint disease, lupus, tendinitis, colitis, ulcerative colitis, asthma, fatty liver disease, inflammatory diseases of the liver and gut, steatohepatitis, atherosclerosis, hyperlipidemia, dyslipidemia, rhinitis, conjunctivitis, oral inflammation, thyroid disease, lung disease, inflammatory bowel disease (IBD), Crohn's disease, chronic obstructive airways disease, multiple sclerosis (MS), chronic inflammatory demyelinating polyneuropathy, Guillain-Barre syndrome, psoriasis, Graves' disease, Hashimoto's disease, thyroiditis, myasthenia gravis, and vasculitis.

19. A compound of Formula (B): 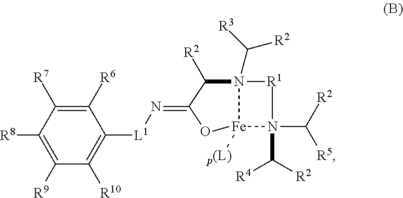
or a pharmaceutically acceptable salt thereof, wherein: [symbol]
indicates a coordinate bond;
each L is independently a Fe ligand;
p is 0, 1 or 2;
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₆ alkyl)_{q}SO₂NR^{A}R^{B}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)O, P(O)(O)(OR^{B}), (C═O)NR^{B}; and ring B, which is optionally substituted with 1, 2, or 3 substituents independently selected from R^{1B};
R⁴ is selected from C(O)O, P(O)(O)(OR^{B}), (C═O)NR^{B}; and ring C, which is optionally substituted with 1, 2, or 3 substituents independently selected from R^{1C};
R⁵ is selected from C(O)O, P(O)(O)(OR^{B}), (C═O)NR^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.

20. The compound of claim 19, wherein the compound has formula: 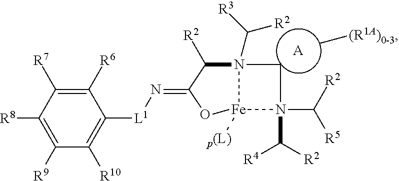
or a pharmaceutically acceptable salt thereof.

21. The compound of claim 19, wherein the compound has formula: 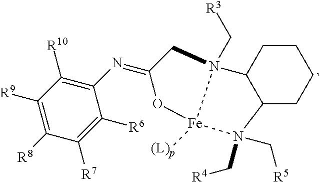
or a pharmaceutically acceptable salt thereof.

22. The compound of claim 19, wherein the compound has formula: 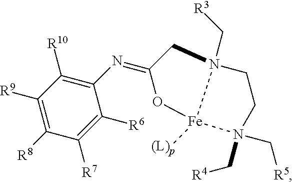
or a pharmaceutically acceptable salt thereof.

23. The compound of claim 19, having formula: 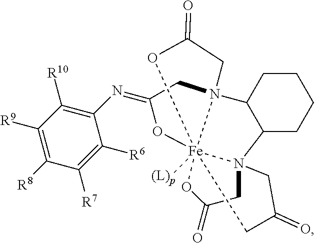
or a pharmaceutically acceptable salt thereof.

24. The compound of claim 19 having formula: 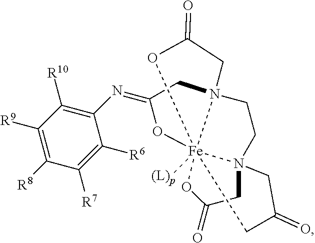
or a pharmaceutically acceptable salt thereof.

25. The compound of a claim 19, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

26. The compound of claim 19, selected from any one of the following compounds: 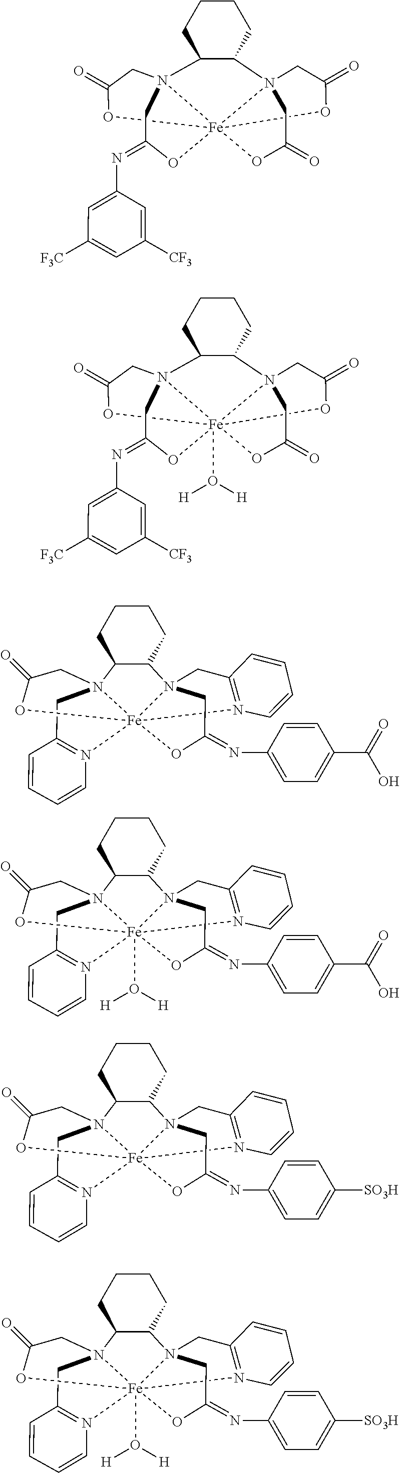 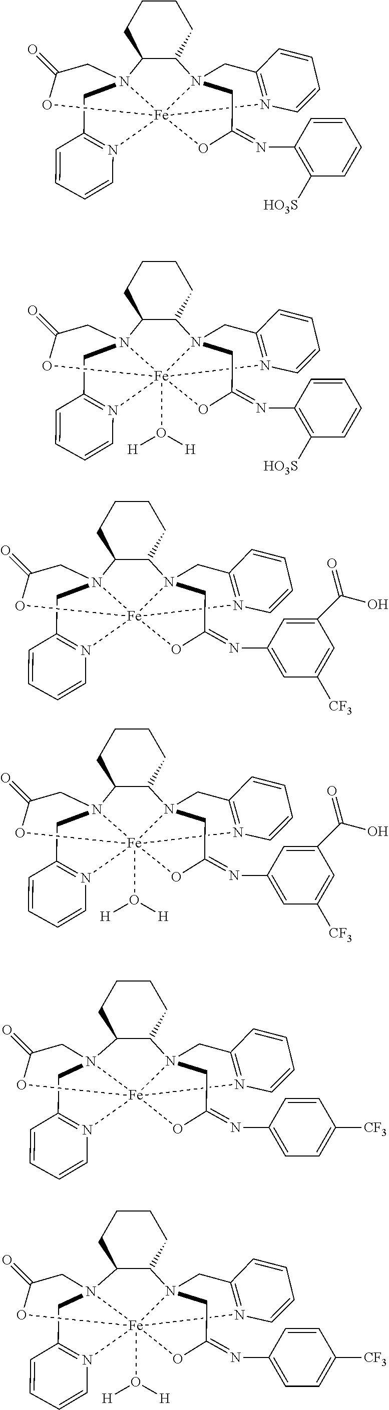 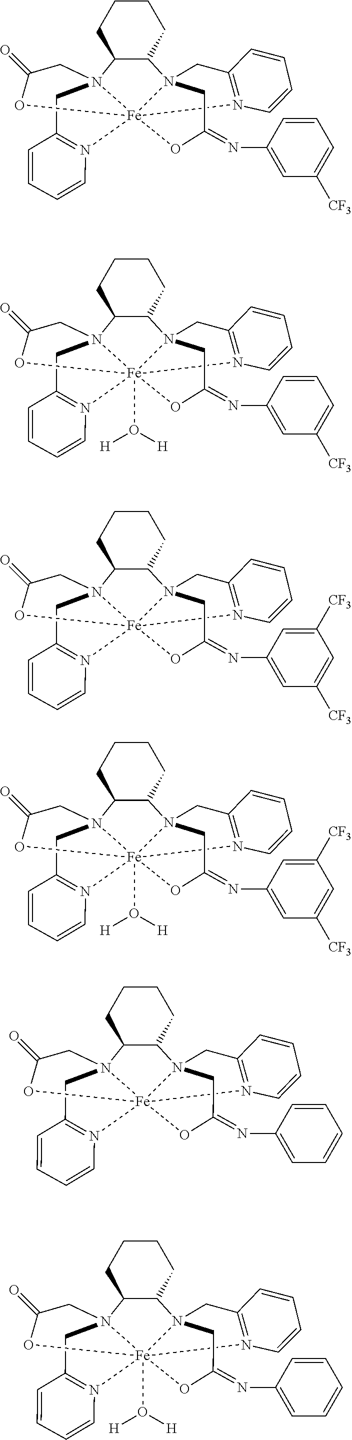 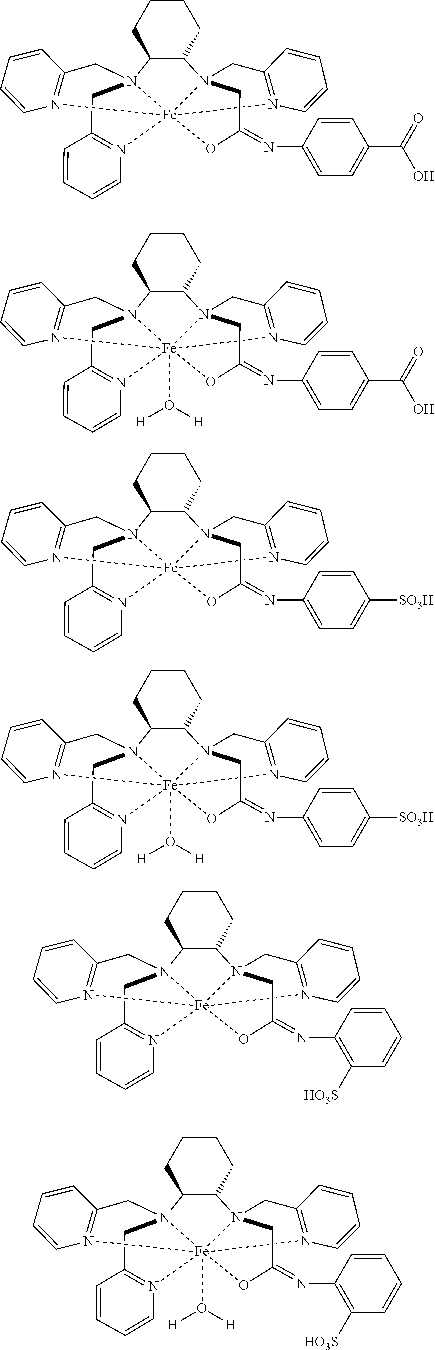 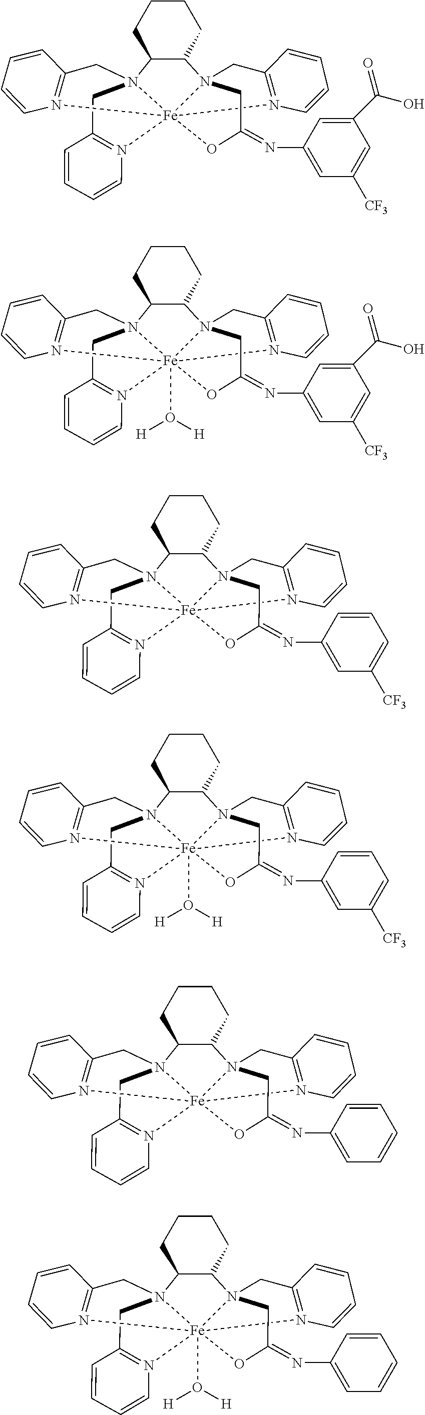 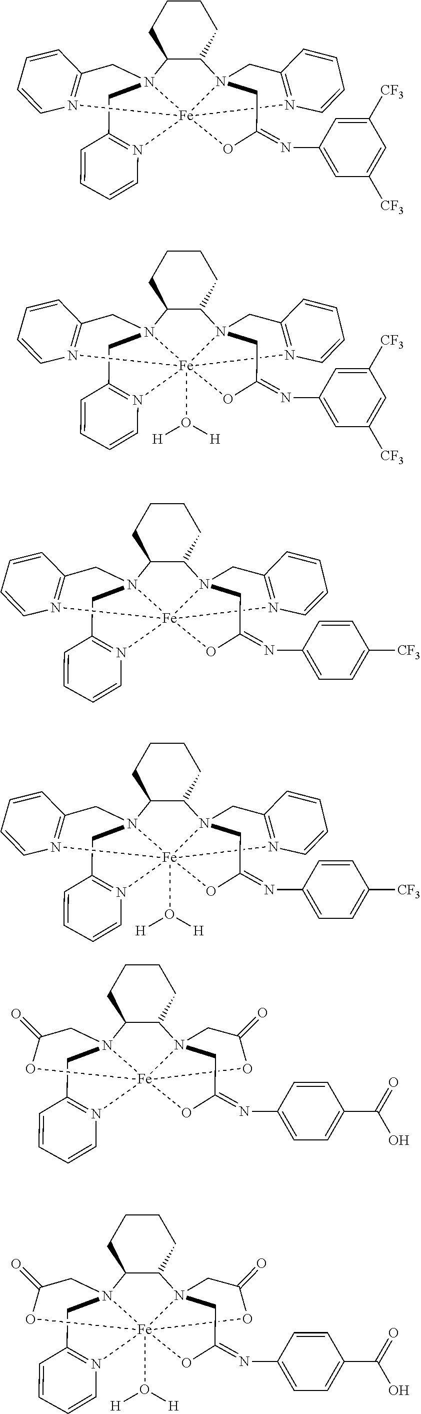 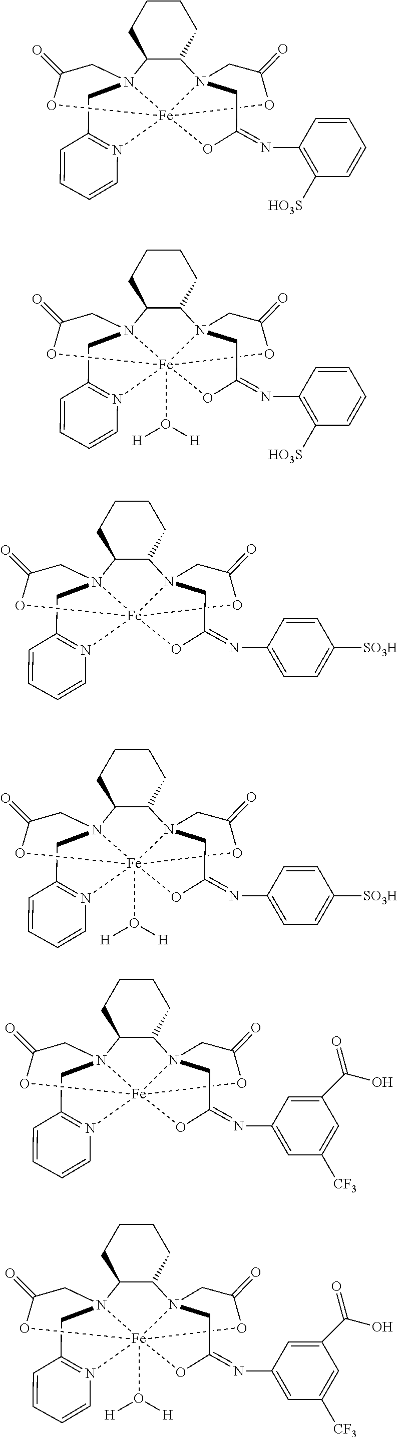 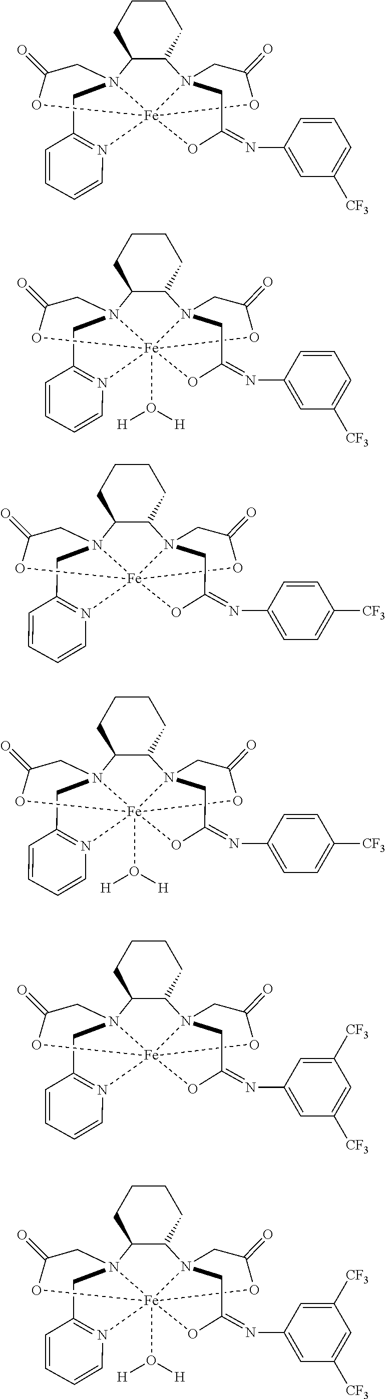 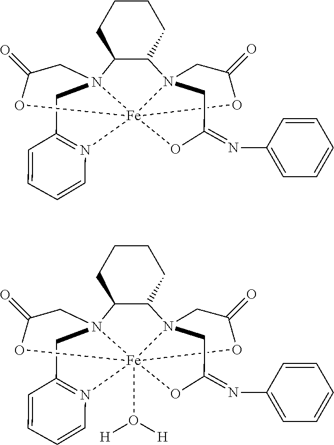
or a pharmaceutically acceptable salt thereof.

27. A pharmaceutical composition comprising a compound of claim 19, or a pharmaceutically acceptable salt thereof, and a pharmaceutically acceptable carrier.

28. A method of magnetic resonance (MR) imaging of an organ or a tissue of a subject, the method comprising:
i) administering to the subject an effective amount of a compound of claim 19, or a pharmaceutically acceptable salt thereof;
ii) waiting a time sufficient to allow the compound to accumulate in the organ or a tissue to be imaged; and
ii) acquiring an MR image of the organ or the tissue of the subject.

29. The method of claim 28, wherein the organ or the tissue is selected from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a cartilage, an epithelium, a tendon, and a ligament.

30. A method of diagnosing a disease or condition in a subject, the method comprising:
i) administering to the subject an effective amount of a compound of claim 19, or a pharmaceutically acceptable salt thereof;
ii) waiting a time sufficient to allow the compound to accumulate in an organ or a tissue of the subject affected by the disease or condition; and
iii) acquiring an MR image of the organ or the tissue of the subject,
wherein observing the MR image attributable to the compound of Formula (B) is indicative of the disease or condition.

31. A method of monitoring treatment of a disease or condition in a subject, the method comprising:
i) administering to the subject an effective amount of a compound of claim 19, or a pharmaceutically acceptable salt thereof;
ii) waiting a time sufficient to allow the compound to accumulate in an organ or tissue of the subject affected by the inflammatory disease or condition;
iii) acquiring a first MR image of the organ or the tissue of the subject;
iv) administering to the subject a therapeutic agent in an effective amount to treat the disease or condition;
v) after iv), administering to the subject an effective amount of a compound of claim 19, or a pharmaceutically acceptable salt thereof;
vi) waiting a time sufficient to allow the compound to accumulate in the organ or tissue of the subject affected by the disease or condition;
vii) acquiring a second MR image of the organ or the tissue of the subject; and
viii) comparing the first and the second MR images,
wherein observing a difference between the first and the second MR images attributable to the compound of Formula (B) is indicative of progression of treatment of the disease or condition.

32. A compound of Formula (I): 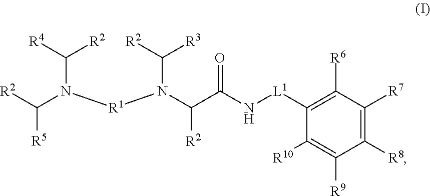
or a pharmaceutically acceptable salt thereof, wherein:
L¹ is C₁₋₃ alkylene; or L¹ is absent;
R¹ is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R¹ is bound to the adjacent nitrogen atoms via the 1,2 or 1,3 positions on R¹;
each ring A is independently selected from C₃-C₁₀ cycloalkylene, 4-10 membered heterocycloalkylene, C₆-C₁₀ arylene, and 5-10 membered heteroarylene;
each R² is independently selected from H, —(C₁₋₃ alkyl)_{q}SO₃R^{A}, —(C₁₋₃ alkyl)_{q}SO₂R^{A}, (C₁₋₃ alkyl)_{q}NHSO₂R^{A}, —(C₁₋₆ alkyl)_{q}SO₂NR^{A}R^{B}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R³ is selected from C(O)OR^{A}, P(O)(OR^{A})(OR^{B}), (C═O)NR^{A}R^{B}; and ring B, which is optionally substituted with 1, 2, or 3 substituents independently selected from R^{1B};
R⁴ is selected from C(O)OR^{A}, P(O)(OR^{A})(OR^{B}), (C═O)NR^{A}R^{B}; and ring C, which is optionally substituted with 1, 2, or 3 substituents independently selected from R^{1C};
R⁵ is selected from CO(OR^{A}), P(O)(OR^{A})(OR^{B}), (C═O)NR^{A}R^{B}; and ring D, which is optionally substituted with 1, 2, or 3 substituents independently selected from R^{1D};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
each R⁶, R⁷, R⁸, R⁹, R¹⁰, R^{1A}, R^{1B}, R^{1C}, and R^{1D} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl;
with a proviso that the compound of Formula (I) is not: 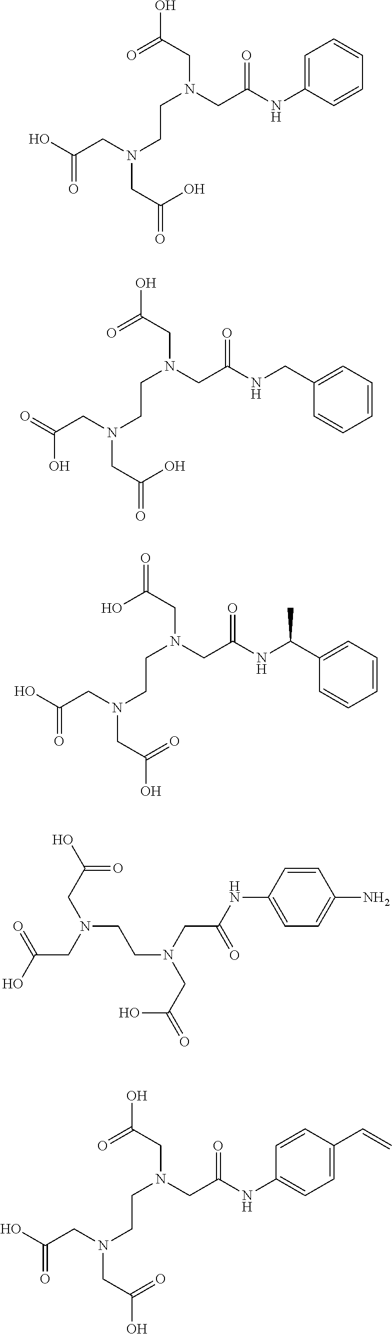 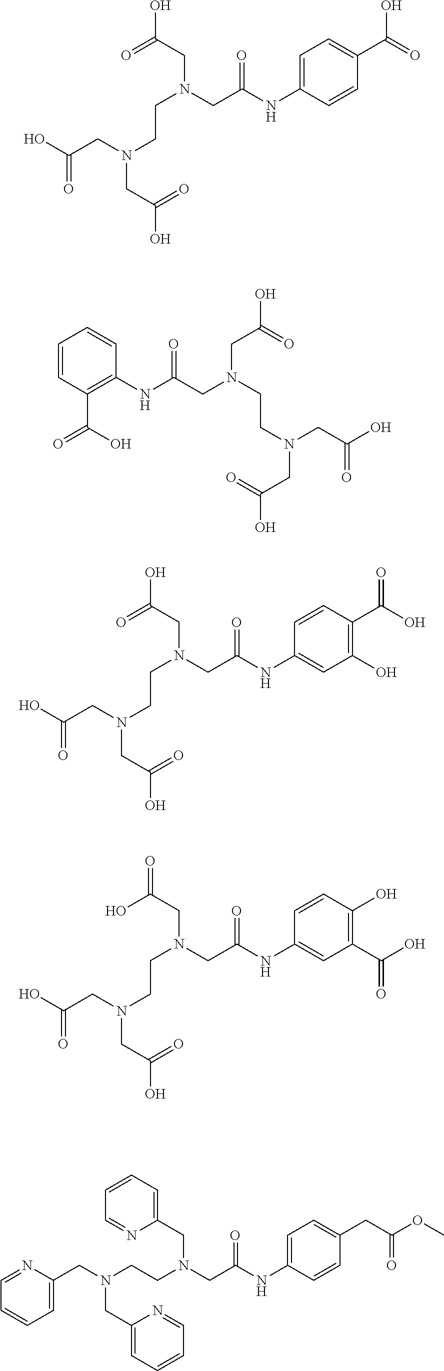 [SMILES: O=C(O)CN(CCN(CC(=O)O)CC(=O)Nc1ccc(C(=O)O)cc1)CC(=O)O ; O=C(O)CN(CCN(CC(=O)O)CC(=O)Nc1ccccc1C(=O)O)CC(=O)O ; O=C(O)CN(CCN(CC(=O)O)CC(=O)Nc1ccc(C(=O)O)c(O)c1)CC(=O)O ; O=C(O)CN(CCN(CC(=O)O)CC(=O)Nc1ccc(O)c(C(=O)O)c1)CC(=O)O ; COC(=O)Cc1ccc(NC(=O)CN(CCN(Cc2ccccn2)Cc2ccccn2)Cc2ccccn2)cc1] 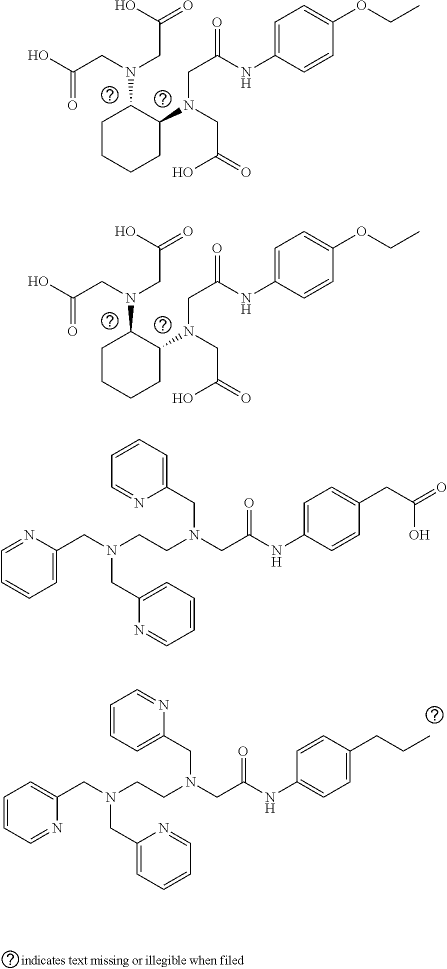
or a pharmaceutically acceptable salt thereof.

33. The compound of claim 32, wherein the compound has formula: 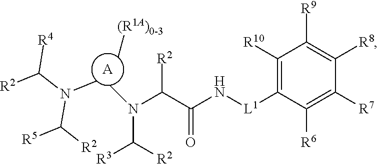
or a pharmaceutically acceptable salt thereof.

34. The compound of claim 32, wherein the compound has formula: 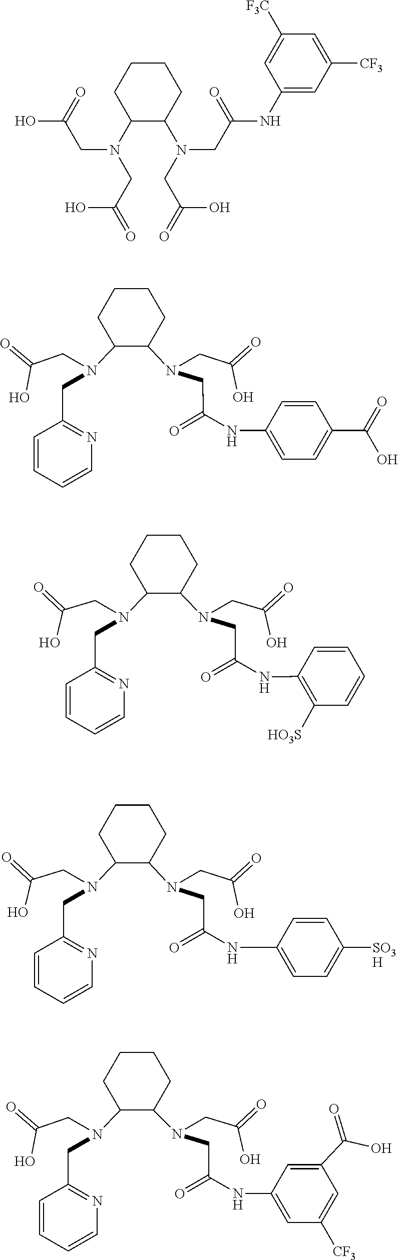 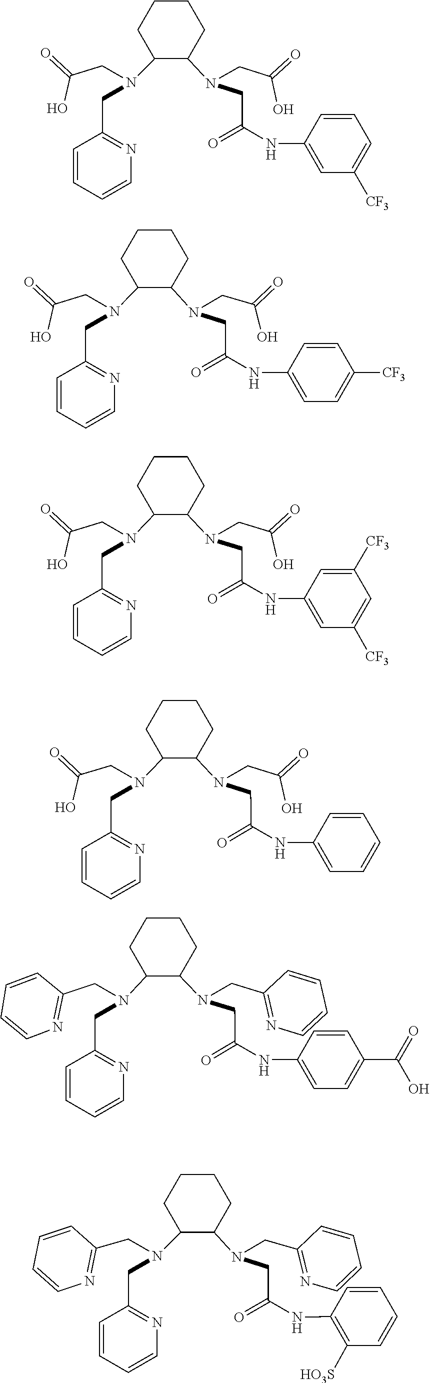 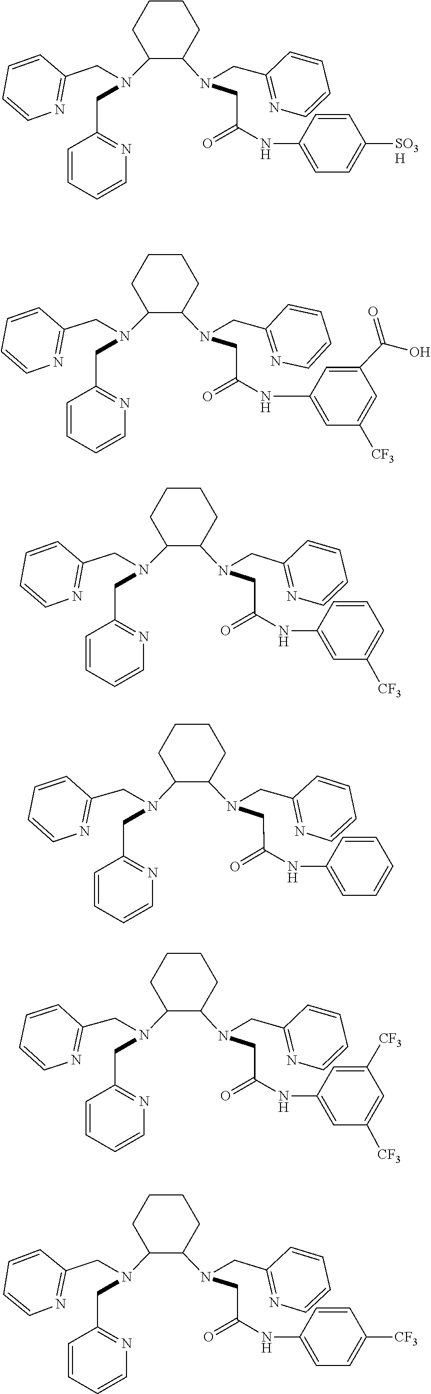 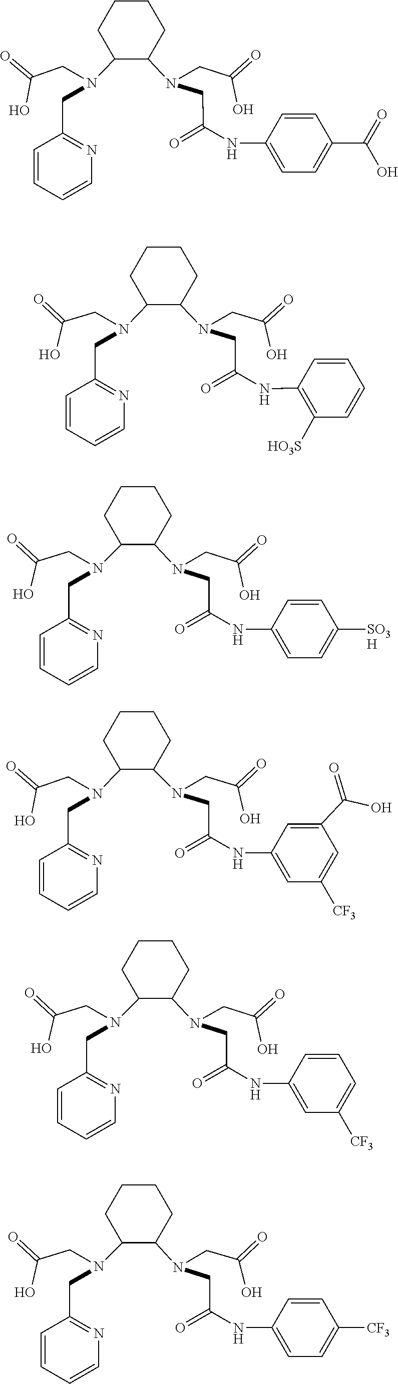 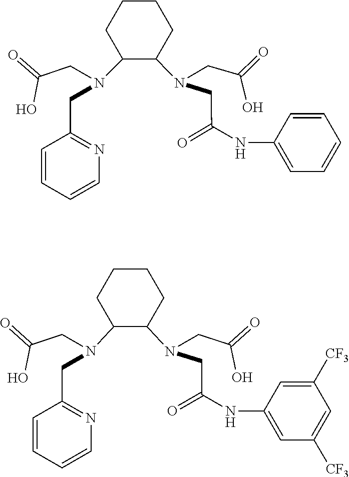
or a pharmaceutically acceptable salt thereof.
